(19) United States
(12) Patent Application Publication
(10) Pub. No.: US 2012/0276132 A1
(43) Pub. Date: Nov. 1, 2012

(54) Atoxic recombinant holotoxins of Clostridium difficile as immunogens

(75) Inventors: Hanping Feng, Ellicott City, MD (US); Haiying Wang, Guangzhou (CN); Saul Tzipori, Shrewsbury, MA (US)

(73) Assignee: TUFTS UNIVERSITY, Boston, MA (US)

(21) Appl. No.: 13/486,550

(22) Filed: Jun. 1, 2012

Related U.S. Application Data
(63) Continuation of application No. PCT/US2010/058701, filed on Dec. 2, 2010.
(60) Provisional application No. 61/265,894, filed on Dec. 2, 2009.

(51) Int. Cl. A61K 39/08; A61P 31/04; C12N 9/52; A61P 37/04; C12N 15/57; C12N 15/63

(52) U.S. Cl. 424/192.1; 424/247.1; 536/23.2; 435/320.1; 435/6.15

(57) ABSTRACT

Atoxic Clostridium difficile toxin proteins were expressed in an endotoxin-free Bacillus system top develop a vaccine to reduce incidence and severity of C. difficile infection (CDI). Immunogens evaluated as potential vaccine candidates are mutated toxin A (encoded by TcdA) and toxin B (TcdB), and a rationally designed chimeric protein containing full-length TcdB protein except that the receptor binding domain is replaced with that of TcdA (designated as cTxAB). A small deletion (97 amino acids) in the transmembrane domain was used to reduce or eliminate toxicity.

### RELATED APPLICATIONS

This application claims the benefit of international application number PCT/US2010/058701 filed Dec. 2, 2010 entitled “Atoxic Recombinant Holotoxins of Clostridium difficile as Immunogens” inventors Hanping Feng, Haiying Wang, and Saul Tzipori, which claims the benefit of U.S. provisional application Ser. No. 61/265,894 filed Dec. 2, 2009 entitled “Methods and Compositions of Atoxic Recombinant Holotoxins of Clostridium difficile as Immunogens, and Kits for Uses Therefor” inventors Hanping Feng, Haiying Wang, and Saul Tzipori, which are hereby incorporated by reference herein in their entireties.

### GOVERNMENT SUPPORT

The invention was made in part under grants K01DK076549, N01AI30050, R01AI088748 and R01DK084509 from the National Institutes of Health. The government has certain rights in the invention.

### TECHNICAL FIELD

The present invention generally relates to immunogenic vaccine compositions derived from atoxic recombinant C. difficile toxin proteins and methods of making and using therefor.

### BACKGROUND

Clostridium difficile, a Gram-positive spore-forming anaerobic bacillus, is the most common cause of nosocomial antibiotic-associated diarrhea and the etiologic agent of pseudomembranous colitis (Cloud, J. et al. 2007 Curr Opin Gastroenterol 23:4-9). The disease ranges from mild diarrhea to life threatening fulminating colitis (Bartlett, J. G. 2002 N Engl J Med 346:334-339; Borriello, S. P. 1998 J Antimicrob Chemother 41 Suppl C:13-194).

C. difficile infection (CDI) is acquired by the ingestion of bacteria or bacterial spores of this strain (Dubberke, E. R. et al. 2007 Am J Infect Control 35:315-318; Roberts, K. et al. 2008 BMC Infect Dis 8:7). Spores survive contact to gastric acidity and germinate in the colon. C. difficile is the most common cause (up to 25%) of hospital acquired and antibiotic associated diarrheas (AAD), and almost all cases of pseudomembranous colitis (Cloud, J. et al. 2007 Curr Opin Gastroenterol 23:4-9).

Antibiotic treatment is a significant risk factor for the diseases, as are advanced age and hospitalization (Bartlett, J. G. 2006 Ann Intern Med 145:758-764). Antibiotic use permits C. difficile which is resistant to most antibiotics to proliferate and produce toxins, as upon antibiotic administrationit does not have to compete with the normal bacterial flora for nutrients (Owens, J. R. et al. 2008 Clinical Infectious Diseases 46:S19-S31). The toxins TcdA and TcdB are the major cause of the disease.

Interventions including administration of probiotics, toxin-absorbing polymers, and toxoid vaccines neither prevent nor treat increasing incidence and seriousness of CDI (Gerding, D. N. et al. 2008 Clin Infect Dis 46 Suppl 1:S32-42). Of further concern is the recent emergence of hypervirulent strains that are resistant to antibiotics.

The incidence of infection is rising steadily (Archibald, L. K. et al. 2004 J Infect Dis 189:1585-1589). Several hospital outbreaks of CDI with high morbidity and mortality which occurred in the last few years in North America have been attributed to the widespread use of broad-spectrum antibiotics. The emergence of new and more virulent C. difficile strains has also contributed to the increased incidence and severity of the disease (Loo, V. G. et al. 2005 N Engl J Med 353:2442-2449; McDonald, L. C. et al. 2005 N Engl J Med 353:2433-2441). Because the surging of the incidence and severity, CDI is now considered an important emerging disease.

According to the US Agency of Healthcare Research and Quality (AHRQ), the incidence of hospital patients infected with CDI jumped 200% from 2000 to 2005, following a 74% increase from 1993 to 2000. Such rapid increases in incidence may be attributed to usage of broad-spectrum antibiotics and/or emergence of new hypervirulent C. difficile strains. Furthermore, most cases of infection occur in patients with risk factors for antibiotic-associated colitis, and an increasing proportion of patients do not have the standard risk factors, including pregnant women, transplant patients, healthcare workers and even previously healthy people living in the community (Severe Clostridium difficile-associated disease in populations previously at low risk—four states. 2005. MMMWR 54:1201-1205).

Standard therapy includes treatment with vancomycin or metronidazole, neither of which is fully effective (Zar, F. et al. 2007 Clinical Infectious Diseases 45:302-307). An estimated 15-35% of those infected with C. difficile relapse following treatment (Barbut, F. et al. 2000 J Clin Microbiol 38:2386-2388; Tonna, I. et al. 2005 Postgrad Med J 81:367-369).

Management of CDI has been estimated to cost the US healthcare system $1.1B each year (Kuijper, E. J. et al. 2006 Clin Microbiol Infect 12 Suppl 6:2-18). The increase in rates of CDI is also associated with heightened disease severity and an increased percentage of colectomies (10.3%) and a higher mortality rate (approximately 25%) than in the past (Dallal, R. M. et al. 2002 Annals of surgery 235:363-372).

The clinical appearance of CDI infection is highly variable, from asymptomatic carriage, to mild self-limiting diarrhea, to the more severe life-threatening pseudomembranous colitis. The most common symptom is diarrhea. Other common clinical symptoms include abdominal pain and cramping, increased temperature and increase in white blood cells. In mild cases of CDI, oral rehydration plus withdrawal of antibiotics is often effective. For CDI cases that are more severe, standard therapy is oral administration of metronidazole or vancomycin is recommended, neither of which is fully effective (Zar, F. et al. 2007 Clinical Infectious Diseases 45:302-307). This treatment is also associated with a relapse rate as high as 55% (Barbut, F. et al. 2000 J Clin Microbiol 38:2386-2388; Walters, B. A. et al. 1983 Gut 24:206-212). Unfortunately, the primary treatment option for recurrent CDI remains metronidazole or vancomycin. Experimental treatments currently in clinical development include toxin-absorbing polymer, some antibiotics, and monoclonal antibodies (Anton, P. M. et al. 2004 Antimicrob Agents Chemother 48:3975-3979; Hinkson, P. L. et al. 2008 Antimicrob Agents Chemother 52:2190-2195; McVay, C. S. et al. 2000 Antimicrob Agents Chemother 44:2254-2258).

There is a need for vaccines that are easily produced and that target TcdA and TcdB to elicit strong systemic and mucosal immunity to prevent CDI, and to reduce severity, eliminate ongoing chronic disease and possibly prevent relapses.

### SUMMARY OF EMBODIMENTS

An embodiment of the invention provided herein is a vaccine composition that includes an atoxic recombinant Clostridium toxin protein for immunizing a subject against infection, the protein having a glucosyltransferase domain (GT), a cysteine proteinase domain (CPD), a transmembrane domain (TMD), and a receptor binding domain (RBD), operably linked to a protein purification tag located at a C-terminus. The composition is effective to immunize the subject. The protein of the composition is produced recombinantly in a Bacillus host. For example, the Bacillus is B. megaterium, B. subtilis or the like. For example, the Clostridium is selected from at least one from the group of: C. difficile, C. perfringens, C. sordellii, C. septicum, C. tertium, C. botulinum, and the like.

The protein in related embodiments includes at least one mutation in at least one toxin protein selected from the group of a C. difficile TcdA protein and a TcdB protein such that the mutation reduces toxicity and retains native protein conformation. In various embodiments, the mutation reduces toxicity at least about 10-fold to about 1,000-fold. For example, the mutation reduces toxicity at least about 10,000-fold to about 10-million fold. In alternative embodiments, the mutation is located in the GT domain of the at least one of the TcdA protein and the TcdB protein. The mutation includes an amino acid substitution or an amino acid deletion. For example, the substitution comprises a replacement of a tryptophan with an alanine or a replacement of an aspartic acid with an asparagine. The protein in various embodiments includes a plurality of mutations.

The composition in an alternative embodiment includes a protein that is a chimeric fusion cTxAB having a first amino acid sequence derived from the TcdA protein and a second amino acid sequence derived from the TcdB protein. For example, the first amino acid sequence includes the TcdA RBD domain and the second amino acid sequence includes the TcdB GT, CPD and TMD domains. The protein domains are operably linked to the purification tag and the protein further includes a protease cleavage site for removal of the tag. The purification tag in related embodiments is at least one selected from the group of: Arg-tag, calmodulin-binding peptide, cellulose-binding domain, DsbA, c-myc-tag, glutathione S-transferase, FLAG-tag, HAT-tag, His-tag, maltose-binding protein, NusA, S-tag, SBP-tag, Strep-tag, and thioredoxin The composition in a related embodiment includes a deletion mutation. For example, the deletion includes at least one aspartic acid in the TMD domain.

An embodiment provides the composition in an effective dose. The composition in related embodiments includes an adjuvant, and/or a pharmaceutically acceptable carrier.

In an alternative embodiment the composition includes a nucleic acid encoding protein, which is operably linked to a bacterial vector.

In alternative embodiments the composition includes either the TcdA protein or the TcdB protein, or both, and the proteins are recombinantly produced separately.

The invention in another embodiment provides a kit that includes a unit dose of a composition according to any of the above embodiments, a container and instructions for use.

An embodiment of the invention herein is a method of eliciting an immune response specific for a Clostridium difficile toxin in a subject, the method including: engineering a nucleic acid encoding an atoxic mutant of a C. difficile toxin protein composition, such that the protein comprises a glucosyltransferase domain (GT), a cysteine proteinase domain (CPD), a transmembrane domain (TMD), a receptor binding domain (RBD), and a purification tag located at a C-terminus; expressing the protein in a Bacillus cell, purifying the protein, and removing the purification tag; and, formulating the composition and contacting the subject with the composition, thereby eliciting in the subject at least one of a humoral immune response and a cell-mediated immune response specific to the protein.

Engineering in a related embodiment includes obtaining a mutation in at least one of a first nucleic acid sequence encoding an amino acid sequence from a TcdA protein, and a second nucleic acid sequence encoding an amino acid sequence from a TcdB protein. For example, the mutation is located in the GT domain of the at least one of the TcdA protein and the TcdB protein. Engineering the mutation in a related embodiment involves introducing an amino acid substitution or an amino acid deletion. For example, the substitution mutation comprises at least one of replacing a tryptophan with an alanine and replacing an aspartic acid with an asparagine. Engineering the protein in an embodiment involves introducing at least one mutation, for example, a plurality of mutations. The method in a related embodiment includes deleting at least one aspartic acid in the TMD domain.

The composition used in the method herein includes an atoxic chimeric protein cTxAB having a first amino acid sequence derived from the TcdA protein and a second amino acid sequence derived from the TcdB protein. Thus, engineering an embodiment of the amino acid sequence includes recombinantly joining nucleic acids encoding the RBD domain from the TcdA protein with that encoding the amino acid sequence of the GT, CPD and TMD domains of the TcdB protein, such that the protein domains are operably linked to a purification tag located at the C-terminus and a protease cleavage site for removal of the tag.

In general, the subject is selected from at least one of the group of: a human, a research animal, a high value zoo animal, and an agricultural animal.

The method in a related embodiment further involves contacting the subject and administering the composition by a route selected from at least one of the group consisting of intravenous, intramuscular, intraperitoneal, intradermal, mucosal, subcutaneous, sublingual, intranasal and oral.

An embodiment of the method further involves analyzing an antibody titer in serum of the subject, and observing an increase in antibody that specifically binds a Clostridium antigen compared to that in a control subject not so contacted, as an indication that the immune response has been elicited in the subject.

An embodiment of the present invention herein provides a method of producing a recombinant mutant Clostridium toxin protein in a Bacillus host, the method including steps: constructing a nucleic acid vector encoding a gene for the Clostridum protein, such that the protein includes a glucosyltransferase domain (GT), a cysteine proteinase domain (CPD), a transmembrane domain (TMD), a receptor binding domain (RBD), such that the gene is operably linked to regulatory signals for expressing the gene in a Bacillus cell and to a selectable marker and to a purification tag located at a C-terminus; contacting a protoplast of a Bacillus cell with the vector; and, selecting a transformant carrying the selectable marker and expressing the recombinant protein in cells of the transformant.

In general, the Bacillus is selected from the group of: B. megaterium, B. subtilis, B. thuringiensis, B. cereus, and B. licheniformis, although other species of bacilli are also envisioned.

In related embodiments of the method the Clostridium protein gene is obtained from at least one from the group of: C. difficile, C. perfringens, C. sordellii, C. septicum, C. tertium, C. botulinum, and the like.

Constructing the nucleic acid vector in a related embodiment involves combining a first nucleic acid sequence encoding an atoxic mutant C. difficile TcdA protein and a second nucleic acid sequence encoding an atoxic mutant C. difficile TcdB protein, for example, ligating the first and second nucleic acids.

The protein in an embodiment of the method includes at least one mutation. For example, the at least one mutation includes a substitution or a deletion of the at least one amino acid. For example, the at least one mutation is located in the GT domain. For example, the at least one mutation comprises a substitution of a tryptophan with an alanine or a substitution of an aspartic acid with an asparagine. In a related embodiment, the protein includes a plurality of mutations.

In an embodiment of the method, the gene encoding a recombinant chimeric cTxAB protein includes a first amino acid sequence derived from the TcdB protein and a second amino acid sequence derived from the TcdA protein. For example, the TcdB protein amino acid sequence includes the GT domain and the TcdA protein amino acid sequence includes the RBD, CPD and TMD domains, such that the protein domains are operably linked to a purification tag with a protease cleavage site for removal of the tag.

In an embodiment of the method, the gene encoding a recombinant chimeric TxB-Ar protein includes a first amino acid sequence derived from the TcdA protein and a second amino acid sequence derived from the Tcd B protein. For example, the TcdA protein amino acid sequence includess the RBD domain and the TcdB protein amino acid sequence includes the GT, CPD and TMD domains, such that the protein domains are operably linked to a purification tag with a protease cleavage site for removal of the tag.

### BRIEF DESCRIPTION OF THE DRAWINGS

FIG. 1 shows an experimental design for immunization and oral bacterial challenge. C57BL/c mice were immunized three times on days −37, −23, and −9, the minus sign denoting days prior to challenge. On day −6, mice were subjected to depletion of intestinal microbiota with 3 days of antibiotic cocktail treatment, then with 1 intraperitoneal (IP) dose of clindamycin on day −1. On day 0, mice were challenged with 10⁴ or 10⁵ CPU of C. difficile. The development of diseases and death was monitored.

FIG. 2 is a set of drawings showing wild type and mutant recombinant toxins.

FIG. 2 panel A shows wild type TcdA (2710 amino acids in length) and TcdB (2366 amino acids in length) expressed in B. megaterium. Both toxins contain functional domains: GT (glucosyltransferase domain), CPD (cysteine protease domain), TMD (transmembrane domain), and RBD (receptor binding domain). A six-amino acid histidine tag was installed in C-terminus of both toxins (Terpe, K Appl Microbiol Biotechnol (2003) 60:523-533). The amino acids W and DXD in GT domain of the toxins in a region substrate (UDP-glucose) binding are indicated. D97 (from amino acid 1754 to 1851) identified to be critical for TcdB activity is indicated as a white band.

FIG. 2 panel B shows mutant holotoxins or toxin chimeric proteins. A1, A2, and aTcdA represent mutant TcdA toxins that have a single (D278N), double (W101A and D278N), and triple (W101A, D278N, and W519A) mutations respectively. The notation aTcdB indicates mutant TcdB having two mutations (W102A and D278N) in the GT domain. cTxAB is TcdB with RBD replaced with the RBD of TcdA. D97 is deleted in this chimeric protein, resulting in cTxAB being completely non-toxic. In addition to the His₍₆₎ tag, a Streptag, was added, a 8-amino acid tag that binds to avidin-like proteins with a high affinity, was installed on the N-terminus of His tag. A thrombin protease cleavage site was fused between the tags and toxin protein, allowing the removal of both tags asappropriate.

FIG. 3 is a set of immunoblots, a Kaplan-Meier plot and photographs showing toxicity and cellular binding of mutant version of toxins.

FIG. 3 panels A and B show immunoblots of CT26 or HT29 cells, respectively. Cells were treated with the indicated concentrations of wild type TcdB or aTcdB for the indicated times, harvested, lysed, and analyzed by immunoblotting using a monoclonal antibody (Clone 102) that only binds to non-glucosylated Rac 1. β-actin was used to control equal sample loading.

FIG. 3 panel C is a set of Kaplan-Meier plots showing survival of Balb/c mice that were IP challenged with 50 or 100 ng of wild type TcdB or with a much higher dose 100 μg of mutant aTcdB administered in 100 μl of PBS intraperitoneally. Mouse survival was monitored and plotted as function of time.

FIG. 3 panel D is a set of photographs showing mouse leukemic monocyte RAW 264.7 cells contacted to medium, TcdA, or cTxAB for 30 minutes at 37° C. Cells were harvested and stained with fluorochrome-conjugated antibody and images were taken using a fluorescent microscope.

FIG. 4 is a set of drawings showing structures of mutant recombinant holotoxins and chimeric proteins.

FIG. 4 panels A and B are drawings showing structures of mutant TcdA and TcdB, respectively. aTcdA shown on panel A represents mutant TcdA with triple (W101A, D287N, and W519A) in GT domain and aTcdB shown on panel B represents mutant TcdB with double mutations (W102A and D288N) in GT domains.

FIG. 4 panel C shows mutant TxB-Ar which is TcdB with its RBD swapped with that of TcdA.

FIG. 4 panel D shows mutant cTxAB, which is TxB-Ar with two mutations (W102A and D288N) in its GT domain. GT: glucosyltransferase domain; CPD: cysteine protease domain; TMD: transmembrane domain; RBD: receptor binding domain; His₍₆₎: 6-histidine tag. The numbers indicate a position of an amino acid residue.

FIG. 5 is a set of line graphs and tables showing results of circular dichroism (CD) structural analysis of wild type and mutant toxins.

FIG. 5 panels A and B show secondary structural analysis of wild type and mutant TcdA (panel A) and TcdB (panel B). Far-UV CD spectra were recorded at 22° C. This structural analysis demonstrated that wild type and mutant GT toxins are structurally similar since the CD spectra are virtually identified.

FIG. 5 panel C are tables of secondary structural composition elements of TcdA (top) and Tcd B proteins (bottom).

FIG. 6 is a set of photographs of immunoblots showing glucosyltransferase activity of the mutant toxins. Vero cell lysates were contacted to 1 μg/ml of wild type or mutant toxin proteins for one or two hours. Rac1 glucosylation was analyzed by immunoblotting using monoclonal antibody (Clone 102) that only binds to non-glucosylated Rac1. β-actin was used as an equal loading control.

FIG. 7 is a set of line graphs showing cytotoxicity of the mutant toxins. Colon carcinoma CT26 cells were contacted to wild or mutant toxins at different concentrations for 72 hours. Toxicity was assayed using MTT (3-(4,5-dimethylthiazol-2-yl)-2,5-diphenyltetrazolium bromide, a yellow tetrazole) and cell viability was expressed as the percentage of surviving cells compared to cells without toxin treatment. The assays were performed three times and data represent mean±s.e.m.

FIG. 7 panel A shows percent survival of CT26 cells in a 96-well plate contacted to aTcdA (closed circle) or TcdA (closed triangle). The data show non-toxicity of aTcdA even at 1000 ng/ml.

FIG. 7 panel B shows percent survival of CT26 cells contacted to aTcdB (closed circle) or TcdB (closed triangle). The data show non-toxicity of aTcdB even at 10,000 ng/ml.

FIG. 8 is a set of Kaplan-Meier plots showing mouse survival as function of time and in vivo toxicity. Balb/c mice were IP challenged with either 100 ng/mouse of wild type (solid lines) TcdA (dark grey line) or TcdB (light grey line), or 100 μg/mouse of mutant (dashed lines) aTcdA (dark grey line) or aTcdB (light grey line). Mouse survival was monitored and the data show that of aTcdA (100 μg) and aTcdB (100 μg), and that wild type TcdA (100 ng) and TcdB (100 ng) were toxic.

FIG. 9 is a line graph showing cytotoxicity of chimeric toxin proteins. CT26 cells in a 96-well plate were contacted to TcdB-Ar (closed triangle) or cTxAB (closed circle) at different concentrations for 72 hours. MTT assays were performed and cell viability was expressed as the percentage of surviving cells compared to cells without toxin treatment. The assays were performed three times and data represent mean±s.e.m.

FIG. 10 is a set of bar graphs showing absence of TLR2 ligands in recombinant toxin preparations after two steps of purification. Monoclonal HEK293 cells expressing human TLR2 were incubated 24 hours with indicated concentrations of recombinant toxin proteins purified by Ni-affinity chromatography and further with a thyroglobulin column for TcdA shown on panel A or a Q-column for TcdB shown on panel B. TLR2 signaling was monitored by expression of a reportor gene (secretory alkaline phosphatase, SEAP) under control of an NF-κB promoter. The amount of SEAP secreted into culture medium was determined by SEAP Reporter Assay (Cat#rep-sap, Invivogen). mTcdA represents a mutant form of rTcdA (A1). Lm (heat-killed Listeria monocytogenes) served as a positive control for the assay. The data show that highly purified recombinant toxin preparations contained little or no TLR2 ligands.

FIG. 11 is a set of photographs of immunoblots showing reactivity of TcdA specific monoclonal antibodies (MAbs).

FIG. 11 panel A shows native purified TcdA at the indicated amounts spotted on a nitrocellulose membrane.

FIG. 11 panel B shows recombinant full-length and truncated TcdA peptide fragments F3 (amino acids 1185 to 1838) and F4 (amino acids 1839 to the C-terminus) separated using a pre-cast gel and transferred onto a nitrocellulose membrane. The membranes were probed with the indicated anti-TcdA MAbs, and protein spots or bands were visualized using a chemiluminescent substrate. BID-555 was purchased from Meridian Life Science, Inc.

FIG. 12 is a bar graph and a set of photographs of histology sections showing TcdA-mediated intestinal inflammation in MyD88^{-/-} mice. MyD88^{-/-} mice were anesthetized and each 4-cm ileal loop was ligated and injected with 50 μg of recombinant TcdA or the same volume of control PBS. The ileal loops were removed four hours later for subsequent histologic analyses.

FIG. 12 panel A is a bar graph showing intestinal fluid accumulation measured by the loop weight (mg) to length (cm) ratio. TcdA caused more than doubling of the weight.

FIG. 12 panel B is a set of photographs of histology sections using hematoxylin and eosin (H&E) staining of sections from TcdA and control PBS treated intestines.

FIG. 12 panel C shows immunohistochemistry staining using antibody specific for myeloperoxidase (MPO).

FIG. 13 is a set of necropsy images of tissue from gnotobiotic piglets inoculated with C. difficile.

FIG. 13 panel A shows an intestinal tract from a piglet inoculated with nontoxigenic strain CD37. The large intestine appeared normal with no mesocolonic edema or inflammation (spiral colon in front of image).

FIG. 13 panel B shows an intestinal tract from a piglet inoculated with toxigenic strain UK6, developing chronic diarrhea of two week duration. The spiral colon (front of image) showed moderate mesocolonic edema and inflammation.

FIG. 14 is a set of bar graphs and Kaplan-Meier plots showing neutralizing titers and protection of subjects against toxin challenge.

FIG. 14 panel A is a set of bar graphs showing neutralizing titers. Serum from each immunized mouse was 2-fold diluted and mixed with TcdB (final concentration 0.25 ng/ml) and was pulsed to CT26 cells. Cell rounding was monitored 24 hours later and neutralizing titers were defined as the reciprocal of the dilutions at which sera lost activity to block cell rounding caused by TcdB. (n=5, data was analyzed via unpaired T test and p=0.008).

FIG. 14 panels B and C are Kaplan-Meier plots showing mouse survival. After three rounds of immunization with toxoid, aTcdB, or a PBS control, mice were challenged IPwith either 100 ng TcdB (panel B) or 200 ng TcdA (panel C). Survival was monitored and data were analyzed by Gehan-Breslow-Wilcoxon test.

FIG. 15 is a set of bar graphs and Kaplan-Meier plots showing that cTxAB immunization induced antibody and protective responses against both TcdA and TcdB. Balb/c mice were immunized IP with cTxAB with alum as adjuvant on day 0, day 10, and day 20.

FIG. 15 panel A is a set of bar graphs showing that immunizations of mice with cTxAB induced IgG antibodies against both TcdA and TcdB. Presera and sera 7-day post each immunization were collected and IgG antibodies against TcdA and TcdB were measured by ELISA using purified native toxin-coated microplates. n=10, the statistical analysis two-way ANOVA was used, and for post test, Bonferroni post-tests was used to compare immunized serum groups with preserum.

FIG. 15 panel B is a Kaplan-Meier plot showing mice survival as function of time. cTxAB immunized mice were challenged IP with either 200 ng or 100 ng of TcdA; PBS-immunized group was challenged with 100 ng of TcdA. Mice immunized with cTxAB entirely survived challenge with 100 ng of TcdA.

FIG. 15 panel C shows mice survival as function of time. Groups of cTxAB- and PBS-immunized mice were IP challenged with 100 ng of TcdB. Mouse survival was monitored and data were analyzed by Gehan-Breslow-Wilcoxon test. Mice immunized with cTxAB entirely survived the challenge.

FIG. 16 is a set of microscopic images showing binding and internalization of TcdA and aTcdA. RAW 264.7 cells were contacted to wild type TcdA (left panel) or to mutant aTcdA (right panel) for 30 minutes at 37° C. Cells were harvested and stained with fluorochrome-conjugated antibody and DAPI. The localization of toxin protein molecules was analyzed by confocal microscopy.

FIG. 17 is a set of bar graphs showing anti-TcdB IgG subtypes in immunized mice. Balb/c mice were immunized IP with 5 μg/injection formalin-inactivated TcdB (toxoid) or aTcdB for three times. Mouse serum samples were collected seven days after the third immunization.

FIG. 17 panel A shows amounts IgG subclasses as optical density at 405 nm (OD) measured using HRP conjugated anti-murine IgG1, IgG2a, IgG2b, IgG2c, and IgG3 secondary antibodies. The pooled sera from each group were diluted 100 or 1000 fold. Data show substantially more IgG induces by aTcdB than toxoid.

FIG. 17 panel B shows the ratios of OD compared to background. Ratios higher than two (indicated by the line) were considered as positive. Pools composed of equal numbers of serum samples from aTcdB-immunized group were serially diluted 2-fold from a 1:500 dilution.

FIG. 18 is a bar graph showing that aTcdB immunization induced a more rapid IgG response than toxoid. Balb/c mice were immunized IPwith 5 μg/injection formalin-inactivated TcdB (toxoid) or to mutant aTcdB on days 0, 7, and 21. Mouse pre-serum (before immunization) and serum samples were collected one week after each immunization, and anti-TcdB IgG was measured by standard ELISA. The data show that aTcdB induced a strong IgG response after the second immunization (n=5).

FIG. 19 is a photograph of an immunoblot showing recognition by aTcdB immunization-induced antibodies of epitopes across the entire length of toxin primary structure. Sera from aTcdB immunized mice were used for immunoblotting each of TcdB and non-overlapping TcdB fragments (from N to C terminus, F1 to F4). The sera were pre-incubated with irrelevant H isTagged recombinant protein to remove possible antibodies specific for Histag.

FIG. 20 is a bar graph comparing aTcdB and toxoid immunization abilities to induce IgG response. Balb/c mice were immunized IP with 5 μg/injection of formalin-inactivated TcdB (toxoid) or aTcdB three times. Mouse serum samples were collected seven days after the third immunization and anti-TcdB IgG was measured by standard ELISA. Data show that aTcdB induced a significantly greater IgG response than toxoid. Data were analyzed with two-way ANOVA and an asterisk indicates the significance between groups (n=5).

FIG. 21 is a set of bar graphs, Kaplan-Meier plots and line graphs showing that chimeric cTxAB immunization induced potent neutralizing antibodies that was specific both for toxins A and B, and protection against oral challenge with C. difficile laboratory strain. Panels A, B and C show assays that were performed at least three times with similar results. Error bars show means±s.e.m.

FIG. 21 panel A is a bar graph showing serum anti-TcdA (open bar) and anti-TcdB (grey bar) IgG titers after cTxAB immunization.

FIG. 21 panel B is a bar graph showing serum anti-TcdA (open bar) and anti-TcdB (grey bar) neutralizing titers after cTxAB immunization.

FIG. 21 panel C is a Kaplan-Meier plot showing survival of control mice treated with the PBS (solid lines) or immunized with cTxAB (dashed lines) Immunized mice were divided into two groups and challenged with lethal doses (100 ng/mouse) of wild type TcdA (light grey lines) or TcdB (dark grey lines) respectively. The data show complete survival of immunized mice.

FIG. 21 panels D-I are sets of Kaplan-Meier plots (P<0.01), line graphs and bar graphs showing mouse survival (panels D and G), weight (panels E and H), and symptoms (diarrhea, panels F and I). Ten days (panels D-F) or three months (panels G-I) after the third immunization cTxAB (grey lines), or control PBS (black lines). Mice were challenged with C. difficile VPI10463 vegetative cells (10⁵ cfu/mouse). Mice were monitored and data were collected and analyzed.

FIG. 22 is a set of bar graphs, Kaplan-Meier plots and line graphs showing antibody response and protection after the immunization with mutant holotoxins in comparison to toxoid.

FIG. 22 panel A is a set of bar graphs showing antibody titers after each immunization with aTcdB (grey bar) or toxoid (open bar) immunization. (* P<0.05) The data show higher titers induced by aTcdB than toxoid

FIG. 22 panel B is a set of bar graphs showing neutralizing titers. Serum from each immunized mouse was serially diluted two-fold and was mixed with wild type TcdB and samples were pulsed to CT26 cells. Cell rounding was monitored 24 hours later and neutralizing titer of each sample, defined as the reciprocal of the dilution at which serum loses activity to block cell rounding caused by TcdB, was determined. (p=0.008).

FIG. 22 panels C and D is a set of Kaplan-Meier plots showing survival ten days after a third immunization with toxoid B (dark grey line), aTcdB (light grey line), or treatment with control PBS (black line). Mice were challenged with 100 ng/mouse of lethal dose of wild type TcdB (panel C) or TcdA (panel D), and survival was monitored and analyzed by the Kaplan-Meier survival curves.

FIG. 22 panels E and F is a set of bar graphs showing serum neutralizing titers for TcdA or TcdB respectively. Mice were immunized with aTcdA, aTcdB, or a mixture of aTcdA and aTcdB, and serum neutralizing titers were measured.

FIG. 22 panels G and H is a set of Kaplan-Meier plots and bar graphs showing mouse survival (panel G) and development of diarrhea (panel H). Groups of mice were immunized with aTcdA (dark grey line), aTcdB (lighter shade of grey line), a mixture of aTcdA and aTcdB (light grey line), or treated with control PBS (black line) three times, and were orally challenged with C. difficile bacteria VPI10463 strain (10⁵ CFU/mouse).

FIG. 22 panels I and J show survival and weight respectively. Groups of mice were injected IP with alpaca anti-sera against TcdA (anti-A, dark grey line), TcdB (anti-B, lighter shade of grey line), or a mixture of TcdA and TcdB (anti-A and anti-B, light grey line; 50 ml each or together) four hours after C. difficile spore (UK1 strain, 10⁶ spores/mouse) inoculation. Control mice were injected with 100 μl of presera (CRT, black line). Mouse survival (panel I) and weight (panel J) were monitored. Data were pooled from two assays (asterisk shows significance between groups of Anti-A+Anti-B and CRT). Data are representative of three independent replicates yielding providing similar results. Error bars show means±s.e.m.

FIG. 23 is a bar graph showing that cTxAB immunization induced antibodies with potent neutralizing activities. Mice were immunized IP four times at ten day intervals and serum samples were collected seven days after the fourth immunization. Serum neutralizing titers were measured using CT26 cells as described herein. The mean (n=4) neutralizing titers against TcdA (12,800) or TcdB (2,600) are shown.

FIG. 24 is a set of Kaplan-Meier curves, line graphs, bar graphs, photographs and schematic drawings showing that cTxAB vaccination reduced or eliminated primary and recurrent CDI induced by a hypervirulent strain.

FIG. 24 panels A-C are a Kaplan-Meier plot, a line graph and a bar graph showing mouse survival (panel A), weight (panel B) and disease symptoms (diarrhea, panel C). After three immunizations, mice were challenged with C. difficile strain UK1 spores (10⁶/mouse; grey line: cTxAB; solid black line: PBS; dashed black line: antibiotic cocktail treatment without spore challenge).

FIG. 24 panels D-G are photographs showing intestinal necropsy and histology of ceca from mice treated with control PBS (panels D and F) or immunized with cTxAB (panels E and G).

FIG. 24 panels H and I is a set of drawings showing schedules of immunization and challenge for CDI relapse/recurrence models.

FIG. 24 panels J-L is a set of a Kaplan-Meier plot, a line graph and a bar graph showing the extent of mouse recurrent disease (panel J, survival curves; panel K, weight; and panel L, diarrhea) after rechallenge according to the schedule shown in panel H.

FIG. 24 panels M-Q is a set of Kaplan-Meier plots, line graphs and a bar graph for mouse primary CDI (panel M, survival curves; panel N, weight loss) and recurrent disease (panel 0, survival curves; panel P, weight loss; and panel Q, diarrhea) after initial challenge and rechallenge according to schedule shown in panel I. Assays were performed at least three times and similar results were obtained. Error bars show means±s.e.m.

FIG. 25 is a table showing toxin shedding after infection in mice immunized with cTxAB or control PBS. Fecal samples from mice immunized with cTxAB or PBS were collected after primary and secondary challenge with C. difficile and toxin activity was measured by standard cytotoxicity assay using CT26 cells. Positive samples that caused cell rounding in 100% of cells after overnight culture, an activity that was neutralized by antitoxin polysera. The data show the percentage of positive mice.

FIG. 26 is a set of bar graphs showing piglet serum antibody response after sublingual (SL) immunization with aTcdB. Piglets were immunized SL four times every other week with 25 μg TcdB with (piglets #1 and #2) or without (piglets #3 and #4) mLT as adjuvant. Serum samples were collected two weeks after each immunization and anti-TcdB antibody responses were measured by ELISA using purified native TcdB-coated plates.

FIG. 27 is a drawing showing preparation of poly-lactide-co-glycoside (PLG) nanoparticles. An aqueous solution of DNA was added to a solution of polymer in CH₂Cl₂ to form nanoparticles; particles were transferred to water-in-oil emulsion and blended with a shear-type mixer; the emulsion was poured into a PVA solution leading to a formation of a water-in-oil-in-water emulsion. After evaporation of CH₂Cl₂, particles were collected for use.

### DETAILED DESCRIPTION

The global emergence of hypervirulent drug-resistant strains and the surge in incidence of Clostridium difficile infection (CDI) represent a major public health concern (Kelly, C P et al. 2008 N Engl J Med 359: 1932; Rupnik, M. H. et al. 2009 Nat Rev Microbiol 7: 526). C. difficile secretes two homologous glucosylating exotoxins TcdA and TcdB that are both pathogenic (Lyras D et al. 2009, Nature 458: 1176; Kuehne, S A et al. 2010 Nature), thus requiring neutralization to prevent disease occurrence.

Examples herein provide vaccines including a parenteral vaccine that induce potent neutralizing antibodies that are specific for both toxins and provide full protection against primary and recurrent CDI in mice. Using a non-pathogenic Bacillus megaterium expression system (Vary P S et al. 2007 Applied microbiology and biotechnology 76: 957; Yang, G et al. 2008 BMC Microbiol 8: 192), glucosyltranferase (GT)-deficient holotoxins were generated and absence of toxicity was demonstrated. The native form of atoxic holotoxin induced significantly more potent anti-toxin neutralizing antibodies than the corresponding toxoid. There was little cross-immunogenicity between TcdA and TcdB. To induce antibodies against both toxins, a clostridial toxin-like chimeric protein was designed by replacing the receptor binding domain of TcdB with that of TcdA and the GT-deficient form was generated and designated cTxAB. Parenteral immunization with this single antigen cTxAB was observed in Examples herein to induce rapid and potent neutralizing antibodies specific for both TcdA and TcdB, conferring complete protection against CDI of both a laboratory and a hypervirulent strain. A murine CDI relapse model was established that showed that this vaccine conferred rapid protection both for primary and recurrent C. difficile infection, thus providing a suitable potential prophylactic vaccine for individuals at high risk of developing CDI.

Clostridium difficile TcdA and TcdB are glucosyltransferases (GT) having an ability to modify host Rho family proteins that causes the primary virulent factor. Serum antibodies specific for the two toxins are associated with protection in patients (Kyne, L et al. 2001 Lancet 357: 189; B. A. Leav, B A et al. 2009 Vaccine 28: 965). Human monoclonal antibodies specific for each of TcdA and TcdB protect CDI patients from relapse (Lowy I et al. 2010 N Engl J Med 362: 197). Therefore, a vaccine inducing neutralizing antibodies against the toxins would likely be useful to prevent the disease or reduce its severity.

Protection against CDI is mediated through systemic and mucosal antibodies against the two toxins, although other virulence attributes are known to exist which may also contribute to manifestation of CDI (Aboudola, S. et al. 2003 Infection and immunity 71:1608-1610). Neutralizing monoclonal antibodies directed against TcdA inhibit fluid secretion in mouse intestinal loops and protect mice against systemic infection (Corthier, G. et al. 1991 Infect Immun 59:1192-1195). Co-administration of both anti-TcdA and anti-TcdB antibodies significantly reduces the mortality in a primary disease hamster model as well as in a less stringent relapse model (Babcock, G. J. et al. 2006 Infect Immun 74:6339-6347). Antibodies against C. difficile are present in the general population in individuals greater than two years of age, and a higher level of serum or mucosal antibody response is associated with less severe disease and less frequent relapse (Babcock, G. J. et al. 2006 Infect Immun 74:6339-6347; Kelly, C. P. et al. 1996 Antimicrob Agents Chemother 40:373-379; Kyne, L. et al. 2000 N Engl J Med 342:390-397). Humanized monoclonal antibodies against the toxins are under clinical trial for treatment of patients with CDAD. However, the mechanism by which serum antibodies prevent enterotoxicity and mucosal damage is not fully understood and antibodies may be susceptible to degradation in the intestines and efficacy therefore compromised. Studies have shown that systemically administered human monoclonal IgG antibodies protect hamsters from acute C. difficile infection, but whether these antibodies can protect against chronic disease is unknown.

A toxoid vaccine has been generated by formaldehyde treatment and administered by intramuscular injection with alum as adjuvant (Kotloff, K. L. et al. 2001 Infect Immun 69:988-995; Sougioultzis, S. et al. 2005 Gastroenterology 128:764-770). Chemically detoxified toxoid induces a poorer mucosal response than molecules that target receptors on mucosal surfaces (Cropley, I. et al. 1995 Vaccine 13:1643-1648; Torres, J. F. et al. 1995 Infect Immun 63:4619-4627), since toxoid is unable to bind to the mucosal surface as a result of formaldehyde treatment (Kunkel, G. R. et al. 1981 Mol Cell Biochem 34:3-13). A vaccine that targets both TcdA and TcdB, and that elicits strong systemic and mucosal immunity to prevent CDI, reduces the severity, or eliminates an ongoing chronic disease is needed.

The aTxAB or cTxAB immunogens are shown herein to be superior to toxoid or fragments thereof. The full-length proteins mimic the native form with correct folding, and were observed to generate a full spectrum of neutralizing systemic and mucosal antibodies. Unlike chemically-detoxified toxoid or fragments that contain a small portion of TcdA, the atoxic holotoxins provided herein and carrying point mutations maintain the same adjuvant activity, antigenicity, and affinity to mucosal epithelium as do native toxins; thus induce greater protective immunity than toxoid and generate a wider spectrum of antibodies than fragments. Immunization with chimeric cTxAB was observed in Examples herein to induce potent protection in mice against lethal challenge with both TcdA and TcdB.

### Disease Manifestation and Therapeutic Approaches

CDI is acquired by the ingestion of vegetative organisms or spores, most likely the latter (Dubberke, E. R. et al. 2007 Am J Infect Control 35:315-318; Roberts, K. et al. 2008 BMC Infect Dis 8:7). Spores survive contact to gastric acidity and germinate in the gut. Antibiotic treatment is the most significant risk factor for the disease (Bartlett, J. G. 2006 Ann Intern Med 145:758-764). The clinical appearance of CDI is highly variable and ranges from asymptomatic to mild self-limiting diarrhea, to more severe pseudomembranous colitis. The most common symptom is diarrhea. Other common clinical symptoms include abdominal pain and cramping, increased temperature and leucocytosis. In mild cases of CDI, oral rehydration and withdrawal of antibiotics is often effective. More severe CDI cases are treated by oral administration of metronidazole or vancomycin.

This treatment however is associated with a relapse rate as high as 55% (Barbut, F. et al. 2000 J Clin Microbiol 38:2386-2388; Walters, B. A. et al. 1983 Gut 24:206-212), and the primary treatment option for recurrent CDI remains metronidazole or vancomycin. Other options, such as probiotics and anion-exchange resins, have limited efficacy and are potentially harmful (Gerding, D. N. et al. 2008 Clin. Infect Dis 46 Suppl 1:S32-42). Experimental treatments in clinical development have included toxin-absorbing polymer, antibiotics, and toxin-specific human monoclonal antibodies (Anton, P. M. et al. 2004 Antimicrob Agents Chemother 48:3975-3979; Hinkson, P. L. et al. 2008 Antimicrob Agents Chemother 52:2190-2195; McVay, C. S. et al. 2000 Antimicrob Agents Chemother 44:2254-2258). A formaldehyde inactivated toxoid vaccine in clinical trial is administered intramuscularly (Kotloff, K. L. et al. 2001 Infect Immun 69:988-995; Sougioultzis, S. et al. 2005 Gastroenterology 128:764-770).

### Virulence Factors

CDI is primarily a toxin-mediated disease. Two extensively studied exotoxins, toxin A (TcdA) and toxin B (TcdB), are thought to be major virulence factors, and C. difficile strains that lack both toxin genes are non-pathogenic both for humans and animals (Elliott, B. et al. 2007 Intern Med J 37:561-568; Kelly, C. P. 1996 Eur J Gastroenterol Hepatol 8:1048-1053; Voth, D. E. et al. 2005 Clin Microbiol Rev 18:247-263). Purified TcdA possesses potent enterotoxic and pro-inflammatory activities, as determined in ligated intestinal loop studies in animals (Kurtz, C. B. et al. 2001 Antimicrobial agents and chemotherapy 45:2340-2347). TcdA is cytotoxic to cultured cells in nanogram quantities. TcdB has been reported to exhibit no enterotoxic activity in animals when administered as pure protein (Lyerly, D. M. et al. 1982 Infection and immunity 35:1147-1150; Lyerly, D. M. et al. 1985 Infect Immun 47:349-352). Isogenic strains that are deficient in each toxin demonstrated that TcdB is a key virulence factor in hamsters (Lyras, D., et al. 2009 Nature 458:1176-1179). Enterotoxic and proinflammatory activities of TcdB were observed form human intestinal xenografts in immunodeficient (SCID) mice (Savidge, T. C. et al. 2003 Gastroenterology 125:413-420). TcdA⁻B⁺ C. difficile strains are associated with pseudomembranous colitis in some patients (Shin, B. M. et al. 2007 Diagn Microbiol Infect Dis. 59:33-37). A small number of C. difficile isolates produce a binary toxin (CDT) that exhibits ADP-ribosyltransferase activity (Blossom, D. B. et al. 2007 Clin Infect Dis 45:222-227; Carter, G. P. et al. 2007 J Bacteriol 189:7290-7301; McMaster-Baxter, N. L. et al. 2007 Pharmacotherapy 27:1029-1039). The role of CDT in development of human disease is not well understood (Stare, B. G. et al. 2007 J Med Microbiol 56:329-335). In addition to toxins, several other factors may play roles in disease manifestation, including fimbriae and other molecules that facilitate adhesion, capsule production and hydrolytic enzyme secretion (Borriello, S. P. 1998 J Antimicrob Chemother 41 Suppl C:13-19). The surface layer proteins of C. difficile are involved in bacterial colonization, and antibodies specific for these proteins are partially protective (Calabi, E. et al. 2002 Infect Immun 70:5770-5778; O'Brien, J. B. et al. 2005 FEMS Microbiol Lett 246:199-205).

### Domains of TcdA and TcdB

TcdA (308 kD) and TcdB (269 kD) belong to a large clostridial cytotoxin (LCT) family and share 49% amino acid identity (Just, I. et al. 2004 Rev Physiol Biochem Pharmacol 152:23-47). The genes tcdA and tcdB and three accessory genes are located on the bacterial chromosome, forming a 19.6-kb pathogenicity locus (PaLoc) (142). TcdA and TcdB are structurally similar to each other (von Eichel-Streiber, C. et al. 1996 Trends Microbiol 4:375-382), consisting of at least three functional domains. The C-terminus contains a receptor binding domain (RBD), has a β-solenoid structure and is involved in receptor binding (Ho, J. G. et al. 2005 Proc Natl Acad Sci USA 102:18373-1837). The middle portion of the toxin primary structure is potentially involved in translocation of the toxin into target cells, and the N-terminus is a catalytic domain having glucosyltransferase activity (Hofmann, F. et al. 1997 J Biol Chem 272:11074-11078). The limits of the three domains have been defined in the literature (Giesemann, T. et al. 2008 J Med Microbiol 57:690-696). The GT domain was defined by expression of recombinant proteins deriving from DNA encoding the amino terminal (Hofmann, F. et al. 1997 J Biol Chem 272:11074-11078). In addition, the GT domain was recovered following cytosolic delivery and N-terminal amino acids determined (Pfeifer, G. et al. 2003 J Biol Chem 278:44535-44541). The crystal structure of RBD of TcdA revealed a solenoid-like structure. The boundary of the RBD in both toxins is near amino acid 1850. Interaction between the C-terminus and the host cell receptors is believed to initiate receptor-mediated endocytosis (Florin, I. et al. 1983 Biochim Biophys Acta 763:383-392; Karlsson, K. A. 1995 Curr Opin Struct Biol 5:622-635; Tucker, K. D. et al. 1991 Infect Immun 59:73-78).

Although the intracellular mode of action remains unclear, it has been proposed that the toxins undergo a conformational change at the low pH of the endosomal compartment, leading to membrane insertion and channel formation (Giesemann, T. et al. 2006 J Biol Chem 281:10808-10815; Qa'Dan, M et al. 2000 Infect Immun 68:2470-2474). A host cofactor trigger sa second structural change accompanied by autocatalytic cleavage and release of the catalytic domain into the cytosol (Pfeifer, G. et al. 2003 J Biol Chem 278:44535-44541; Reineke, J. et al. 2007 Nature 446:415-419). In the cytosol, the catalytic domain of toxins mono-0 glucosylates low molecular mass GTPase of the Rho family, including Rho, Rac, and CDC42 (Just, I. et al. 1995 Nature 375:500-503). Glucosylation of Rho proteins inhibits the molecular switch function, blocking Rho GTPase-dependent signaling in intestinal epithelial cells, leading to alterations in the actin cytoskeleton, massive fluid secretion, acute inflammation and necrosis of the colonic mucosa (Just, I. et al. 1995 Nature 375:500-503; Pothoulakis, C. et al. 2001 Am J Physiol Gastrointest Liver Physiol 280:G178-183).

### Epidemiology and Diagnosis

The incidence of C. difficile in healthy adults is 3-5%, and as high as 60% in healthy neonates and infants (Larson, H. E. et al. 1982 J Infect Dis 146:727-733; Viscidi, R. et al. 1981 Gastroenterology 81:5-9). Despite the high carriage rate in neonates, symptomatic disease is uncommon (McFarland, L. V. et al. 2000 J Pediatr Gastroenterol Nutr 31:220-231). In adults with antibiotic usage and hospitalization, the rate of colonization increases substantially to 20-40% (Bartlett, J. G. 2006 Ann Intern Med 145:758-764). The standard test for infection is detection of C. difficile toxins in stool. Assays include cell culture-based cytotoxicity assay (Bartlett, J. G. et al. 1978 N Engl J Med 298:531-534) and enzyme immunoassays (EIAs; Russmann, H. et al. 2007 Eur J Clin Microbiol Infect Dis 26:115-119; Staneck, J. L. et al. 1996 J Clin Microbiol 34:2718-2721) to detect TcdA and/or TcdB in stool samples. Alternative detection methods include anaerobic culture of bacteria and detecting the bacterial antigen glutamate dehydrogenase (GDH).

### Pharmaceutical Compositions

An aspect of the present invention provides pharmaceutical compositions, wherein these compositions comprise an antigen from a toxin of C. difficile, and optionally further include an adjuvant, and optionally further include a pharmaceutically acceptable carrier.

In certain embodiments, these compositions optionally further comprise one or more additional therapeutic agents. In certain embodiments, the additional therapeutic agent or agents are selected from the group consisting of antibiotics particularly antibacterial compounds, anti-viral compounds, anti-fungals, and include one or more of growth factors, anti-inflammatory agents, vasopressor agents, collagenase inhibitors, topical steroids, matrix metalloproteinase inhibitors, ascorbates, angiotensin II, angiotensin III, calreticulin, tetracyclines, fibronectin, collagen, thrombospondin, transforming growth factors (TGF), keratinocyte growth factor (KGF), fibroblast growth factor (FGF), insulin-like growth factors (IGF), epidermal growth factor (EGF), platelet derived growth factor (PDGF), neu differentiation factor (NDF), hepatocyte growth factor (HGF), and hyaluronic acid.

As used herein, the term “pharmaceutically acceptable carrier” includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences Ed. by German), Mack Publishing, Easton, Pa., 1995 discloses various carriers used in formulating pharmaceutical compositions and known techniques for the preparation thereof. Carriers are selected to prolong dwell time for example following any route of administration, including IP, IV, subcutaneous, mucosal, sublingual, inhalation or other form of intranasal administration, or other route of administration.

Some examples of materials that can serve as pharmaceutically acceptable carriers include, but are not limited to, sugars such as glucose, and sucrose; starches such as corn starch and potato starch; cellulose and its derivatives such as sodium carboxymethyl cellulose, ethyl cellulose, and cellulose acetate; powdered tragacanth; malt; gelatin; talc; excipients such as cocoa butter and suppository waxes; oils such as peanut oil, cottonseed oil, safflower oil, sesame oil, olive oil, corn oil, and soybean oil; glycols such as propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

In yet another aspect, according to the methods of treatment of the present invention, the immunization is promoted by contacting the subject with a pharmaceutical composition, as described herein. Thus, the invention provides methods for immunization comprising administering a therapeutically effective amount of a pharmaceutical composition comprising active agents that include an immunogenic toxin protein of C. dificile having an associated antigenic determinant for at least one of TcdA and TcdB, to a subject in need thereof, in such amounts and for such time as is necessary to achieve the desired result. It will be appreciated that this encompasses administering an inventive vaccine as described herein, as a preventive or therapeutic measure to promote immunity to infection by C. dificile, to minimize complications associated with the slow development of immunity (especially in compromised patients such as those who are nutritionally challenged, or at risk patients such as the elderly or infants).

In certain embodiments of the present invention a “therapeutically effective amount” of the pharmaceutical composition is that amount effective for promoting appearance of antibodies in serum specific for the toxins of C. dificile, or disappearance of disease symptoms, such as amount of antigen or toxin or bacterial cells in feces or in bodily fluids or in other secreted products. The compositions, according to the method of the present invention, may be administered using any amount and any route of administration effective for generating an antibody response. Thus, the expression “amount effective for promoting immunity”, as used herein, refers to a sufficient amount of composition to result in antibody production or remediation of a disease symptom characteristic of infection by C. difficile.

The exact dosage is chosen by the individual physician in view of the patient to be treated. Dosage and administration are adjusted to provide sufficient levels of the active agent(s) or to maintain the desired effect. Additional factors which may be taken into account include the severity of the disease state, e.g., contact to infectious agent in the past or potential future contact; age, weight and gender of the patient; diet, time and frequency of administration; drug combinations; reaction sensitivities; and tolerance/response to therapy. Long acting pharmaceutical compositions might be administered every 3 to 4 days, every week, or once every two weeks depending on half-life and clearance rate of the particular composition.

The active agents of the invention are preferably formulated in dosage unit form for ease of administration and uniformity of dosage. The expression “dosage unit form” as used herein refers to a physically discrete unit of active agent appropriate for one dose to be administered to the patient to be treated. It will be understood, however, that the total daily usage of the compositions of the present invention will be decided by the attending physician within the scope of sound medical judgment. For any active agent, the therapeutically effective dose can be estimated initially either in cell culture assays or in animal models, usually mice, rabbits, dogs, or pigs or piglets or other suitable animals. The animal models described herein including that of chronic or recurring infection by C. difficile is also used to achieve a desirable concentration range and route of administration. Such information can then be used to determine useful doses and routes for administration in humans.

A therapeutically effective dose refers to that amount of active agent which ameliorates at least one symptom or condition. Therapeutic efficacy and toxicity of active agents can be determined by standard pharmaceutical procedures in cell cultures or experimental animals, e.g., ED50 (the dose is therapeutically effective in 50% of the population) and LD50 (the dose is lethal to 50% of the population). The dose ratio of toxic to therapeutic effects is the therapeutic index, and it can be expressed as the ratio, LD50/ED50. Pharmaceutical compositions which exhibit large therapeutic indices are preferred. The data obtained from cell culture assays and from animal studies are used in formulating a range of dosage for human use.

The therapeutic dose shown in examples herein is at least about 1 μg per kg, at least about 5, 10, 50, 100, 500 μg per kg, at least about 1 mg/kg, 5, 10, 50 or 100 mg/kg body weight of the purified toxin vaccine per body weight of the subject, although the doses may be more or less depending on age, health status, history of prior infection, and immune status of the subject as would be known by one of skill in the art of immunization. Doses may be divided or unitary per day and may be administered once or repeated at appropriate intervals.

### Administration of Pharmaceutical Compositions

After formulation with an appropriate pharmaceutically acceptable carrier in a desired dosage, the pharmaceutical compositions of this invention can be administered to humans and other mammals topically (as by powders, ointments, or drops), orally, rectally, mucosally, sublingually, parenterally, intracisternally, intravaginally, intraperitoneally, bucally, sublingually, ocularly, or intranasally, depending on preventive or therapeutic objectives and the severity and nature of a pre-existing infection.

In various embodiments of the invention herein, it was observed that high titers of antibodies, sufficient for protection against a lethal dose of C. difficile toxin, were produced after administration of the engineered atoxic toxin proteins provided herein. Liquid dosage forms for oral administration include, but are not limited to, pharmaceutically acceptable emulsions, microemulsions, solutions, suspensions, syrups and elixirs. In addition to the active agent(s), the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Dosage forms for topical or transdermal administration of an inventive pharmaceutical composition include ointments, pastes, creams, lotions, gels, powders, solutions, sprays, inhalants, or patches. The active agent is admixed under sterile conditions with a pharmaceutically acceptable carrier and any needed preservatives or buffers as may be required. Administration may be therapeutic or it may be prophylactic.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables. The injectable formulations can be sterilized prior to addition of spores, for example, by filtration through a bacterial-retaining filter, or by incorporating sterilizing agents in the form of sterile solid compositions which can be dissolved or dispersed in sterile water or other sterile injectable medium prior to use. In order to prolong the effect of an active agent, it is often desirable to slow the absorption of the agent from subcutaneous or intramuscular injection. Delayed absorption of a parenterally administered active agent may be accomplished by dissolving or suspending the agent in an oil vehicle. Injectable depot forms are made by forming microencapsule matrices of the agent in biodegradable polymers such as polylactide-polyglycolide. Depending upon the ratio of active agent to polymer and the nature of the particular polymer employed, the rate of active agent release can be controlled. Examples of other biodegradable polymers include poly(orthoesters) and poly(anhydrides). Depot injectable formulations are also prepared by entrapping the agent in liposomes or microemulsions which are compatible with body tissues.

Compositions for rectal or vaginal administration are preferably suppositories which can be prepared by mixing the active agent(s) of this invention with suitable non-irritating excipients or carriers such as cocoa butter, polyethylene glycol or a suppository wax which are solid at ambient temperature but liquid at body temperature and therefore melt in the rectum or vaginal cavity and release the active agent(s).

Solid dosage forms for oral, mucosal or sublingual administration include capsules, tablets, pills, powders, and granules. In such solid dosage forms, the active agent is mixed with at least one inert, pharmaceutically acceptable excipient or carrier such as sodium citrate or dicalcium phosphate and/or a) fillers or extenders such as starches, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof.

Solid compositions of a similar type may also be employed as fillers in soft and hard-filled gelatin capsules using such excipients as milk sugar as well as high molecular weight polyethylene glycols and the like. The solid dosage forms of tablets, dragees, capsules, pills, and granules can be prepared with coatings and shells such as enteric coatings, release controlling coatings and other coatings well known in the pharmaceutical formulating art. In such solid dosage forms the active agent(s) may be admixed with at least one inert diluent such as sucrose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active agent(s) only, or preferentially, in a certain part of the intestinal tract, optionally, in a delayed manner. Examples of embedding compositions which can be used include polymeric substances and waxes.

### Identifying Routes of Immunization

Routes suitable for inducing systemic and mucosal antibodies and protective responses are identified by comparing oral, intranasal (IN), or sublingual (SL) immunization regimens, with a view to establish systemic protection levels similar or superior to IP immunization as well as mucosal protection.

Both aTxAB and cTxAB contain intact receptor binding domain(s) of native toxins, and are likely have similar affinities for epithelial cells as do wild type toxins. Consequently, mucosal immunity is induced by contact of mucosal surfaces (oral, IN, or SL) to these immunogens. Several routes of mucosal immunizations are compared and the induction of systemic and mucosal antibody responses was assessed. The usage of mucosal adjuvants is also evaluated.

Beginning with the optimal dose established as described in Examples herein, groups of mice are immunized three times with aTxAB or cTxAB, IN or SL (with or without mucosal adjuvant), or orally (encapsulated). Serum and fecal antibody responses are measured after each immunization. One week after the last immunization mice ware challenged IP with the corresponding LD₅₀ᵢ toxin established, and the protective responses to systemic challenge are compared with groups immunized IP and with a placebo group. Systemic antibodies in serum and secretory IgA and IgG against toxins in feces and gut contents are measured and neutralizing titers for blocking cytototoxicity in cell culture are determined. Mucosal protection is evaluated in the ligated ileal loops of immunized mice directly injected with toxin. Dose optimization of the immunogen(s), combined with alternate mucosal adjuvants, follows if the levels of antibody and protective responses are considerably less than that accomplished by parenteral immunization, and/or if the mucosal antibody and protective responses are considered to be low. These assays establish whether mucosal immunization is efficient in terms of antibody and protective responses as IP immunization, and whether a protective mucosal immunity is induced by mucosal immunization.

### Alum as Adjuvant

Intraperitoneal (IP) immunization with aTcdB or cTxAB using alum as adjuvant was shown to induce strong IgG response and systemic protection. Importantly, alum is an FDA approved adjuvant for human vaccination. Therefore, parenteral immunizations included alum as adjuvant, including the placebo.

### Mucosal Adjuvants

The bacterial enterotoxins cholera toxin (CT) from Vibrio cholerae and the heat labile toxin (LT) from E. coli are probably the most commonly used mucosal adjuvants, boosting immune responses to unrelated antigens co-administered by oral or nasal routes (Rappuoli, R. et al. 1999 Immunol Today 20:493-0.500). However, the wild types of these enteric toxins are toxic, therefore, extensive studies have been carried to reduce the toxicity of CT and LT while retain their adjuvant acitivities (Pizza, M. et al. 2001 Vaccine 19:2534-2541). An example is the mutant LT (mLT) which carries a mutation in the proteolytic site of the A subunit at amino acid 192 that abrogates cleavage and attenuates the toxicity of the protein (Dickinson, B. L. et al. 1995 Infect Immun 63:1617-1623). While these adjuvants are important for boosting mucosal immune response, both aTxAB and cTxAB contain intact TBD of TcdA, which possesses adjuvant activity as strong as CT or LT after intranasal administration (Cavalcante, I. C. et al. 2006 Infect Immun 74:2606-2612). In addition, both toxins have high affinity to epithelial cells, thus an optimal dose of these immunogens, without extraneous adjuvant, may be sufficient to induce strong neutralizing IgG and IgA responses.

Comparisons were made between groups of animals immunized with immunogens lacking adjuvant, or including mutant LT (mLT) or mutant CpG. mLT has been used in animal and in human studies (Dickinson, B. L. et al. 1995 Infect Immun 63:1617-1623; Uddowla, S. et al. 2007 Vaccine 25:7984-7993). mLT was constructed using site-directed mutagenesis to create a single amino acid substitution within the disulfides subtended region of the A subunit separating A1 from A2 (Dubberke, E. R. et al. 2007 Am J Infect Control 35:315-318). This single amino acid change altered the proteolytically sensitive site within this region, rendering the mutant insensitive to trypsin activation. The outcome of immunization of each immunogen mixed with 5 μg or 10 μg of mLT was compared and results are presented in the Examples herein.

The immunomodulatory properties of CpGs (Kindrachuk, J. et al. 2009 Vaccine 27:4662-4671) used herein include those useful for a number of potential medical applications: priming the innate immune response, as anti-allergens, for the treatment of a variety of malignancies, and as adjuvants for improving vaccination efficiency, especially in individuals with poor immune responses. Indeed these molecules have been demonstrated to enhance human, murine, and porcine neonatal immune responses, although the use of CpGs in adjuvant formulations was previously demonstrated to skew vaccine-induced immune responses towards a Th1-bias (Garlapati, S. et al. 2009 Vet Immunol Immunopathol 128:184-191). In the context of a vaccine adjuvant, a balanced Th1/Th2 response is desirable since the modulation of Th1 and Th2 contributions influences the balance between protection and immunopathology (Singh, V. K. et al. 1999 Immunol Res 20:147-161).

### Intranasal Immunization (IN)

The mucosal nasal route of immunization induces an immune response resulting in systemic and/or mucosal antibody response in mice, and in the intestines in humans (Kozlowski, P. A. et al. 2002 J Immunol 169:566-574; Rudin, A. et al. 1999 Infect Immun 67:2884-2890). The nasal route avoids protein digestion and degradation in the GI tract, allowing far less antigen to be delivered than the oral route (Kozlowski, P. A. et al. 2002 J Immunol 169:566-574). Therefore, the nasal route of immunization is considered herein to have a great potential (Neutra, M. R. et al. 2006 Nat Rev Immunol 6:148-158).

For intranasal route of immunization, 5 μl of PBS containing aTxAB or cTxAB with or without adjuvant is delivered into each nostril (total 10 μl per mouse). The volume of 5 μl per nostril ensures that all immunogens are distributed inside of nasal cavity. Higher volumes, such as 30 μl, may lead to nasal/pulmonary immunization (Southam, D. S. et al. 2002 Am J Physiol Lung Cell Mol Physiol 282:L833-839). Binding of the immunogens to nasal epithelium is evaluated. The use of LT as adjuvant alters antigen trafficking in the nasal tract. This is the case with wild type LT but not mLT, since this adjuvant-dependent redirection of antigen is dependent on ADP-ribosyltransferase activity (van Ginkel, F. W. et al. 2005 Infect Immun 73:6892-6902).

### Sublingual Immunization (SL)

The SL route has been used for many years to deliver low molecular weight drugs to the bloodstream (Zhang, H. et al. 2002 Clin Pharmacokinet 41:661-680) and for immunotherapy directed towards allergens (O'Hehir, R. E. et al. 2007 Curr Med Chem 14:2235-2244). This route of vaccination has a potential for ease of delivery and for inducing broad systemic and mucosal immune response (Cuburu, N. et al. 2007 Vaccine 25:8598-8610). SL immunization induces intestinal mucosal immunity against infection with enteric pathogens (Huang, C. F. et al. 2008 J Pediatr Gastroenterol Nutr 46:262-271). Gnotobiotic piglets immunized with aTcdB using mutant LT as adjuvant induced higher level of anti-TcdB IgG in circulation than did aTcdB alone as shown in Examples herein. For all these reasons, SL route of immunization was included for evaluation.

The sublingual mucosa encompasses the ventral side of the tongue and the floor of the mouth. For SL immunization, mice are anesthetized with ketamine/xylazine, and 5 μl of aTxAB or cTxAB with or without adjuvant is delivered at the ventral side of the tongue and directed toward the floor of the mouth. Animals are maintained with heads placed in anteflexion for 30 minutes.

### Oral Immunization

Direct stimulation of the gut mucosa induces effective protection against enteric infections. Attempts to deliver inactivated or subunit vaccines of particles, proteins or DNA have been tried by many groups with mixed results. Oral vaccination is safes and effective method for protecting the gut against infection. It is also treacherous route because of proteolytic or hydrolyzing digestive enzymes, bile salts, and extreme pH as well as rapid movement of contents and often limited access to the mucosal wall.

PLG polymers were selected for use in Examples herein because the polymers used for encapsulation are non-immunogenic and have a known record of safety. This has been shown in their use for other purposes, such as in drug delivery and in surgical suture materials. Poly (lactide-co-glycolide) is hydrolyzed in vivo to two naturally occurring substances, lactic acid and glycolic acid.

### Uses of Pharmaceutical Compositions

As discussed above and described in greater detail in the Examples, engineered toxin proteins are provided herein that are effective in eliciting antibody production for toxins of C. dificile and for preventing disease symptoms, infection, and death. In general, it is believed that these vaccines will be clinically useful in immunizing subjects for resistance to CDT. The vaccines herein are particularly useful to treat compromised patients, particularly those anticipating therapy involving, for example, immunosuppression and complications associated with systemic treatment with steroids, radiation therapy, non-steroidal anti-inflammatory drugs (NSAID), anti-neoplastic drugs and anti-metabolites. Patients receiving large routine doses of antibiotic therapy which is known to eliminate or reduce intestinal flora, for example surgical patients and those experiencing trauma such as arising from accidents or battle field wounds, are populations that can be immunized to prevent development of CDI as C. dificile flourishes absent competing normal bacterial flora. It is envisioned also that the vaccines herein may be used prophylactically to immunize entire populations such as school age children or members of the military for prevention of CDI, particularly after catastrophes such as earthquakes and floods.

### Systemic and Mucosal Antibodies in Protection Against CDI

Both systemic and mucosal immunity provide protection against enteric pathogens and pathogenic products, such as toxins (Huang, C. F. et al. 2008 J Pediatr Gastroenterol Nutr 46:262-271; Perez, J. L. et al. 2009 Vaccine 27:205-212). Because TcdA and TcdB are essential virulence factors for C. difficile, an antitoxin preparation can convey full protection from oral C. difficile challenge in animals (Kink, J. A. et al. 1998 Infect Immun 66:2018-2025; Lyerly, D. M. et al. 1991 Infect Immun 59:2215-2218). Antibodies against both toxins, but not against TcdA or TcdB alone, protect against toxigenic C. difficile infection in a hamster model (Fernie, D. S. et al. 1983 Dev Biol Stand 53:325-332; Kim, P. H. et al. 1987 Infect Immun 55:2984-2992; Libby, J. M. et al. 1982 Infect Immun 36:822-829). An evaluation of the routes of delivery of toxoid vaccine in hamsters assessing protection from both lethal disease and diarrhea have found that a combination of mucosal and parental immunization provided complete protection from diarrhea and death, showing that induction of both systemic and mucosal immunity was necessary for optimal protection (Tones, J. F. et al. 1995 Infect Immun 63:4619-4627). The systemic administrated human monoclonal IgG antibodies protected hamsters from acute CDI and mortality (Babcock, G. J. et al. 2006 Infect Immun 74:6339-6347). Whether these antibodies can protect against chronic diseases is unknown. Since these are administered systemically and are passively acquired antibodies, the duration of protection is limited and costly.

In humans, a high level of antitoxin antibodies in serum is associated with less severe disease and less frequent relapses (Kyne, L. et al. 2000 N Engl J Med 342:390-397). Following symptomatic infection, most individuals develop antibodies against the two toxins in serum (Aronsson, B. et al. 1985 Infection 13:97-101; Viscidi, R. et al. 1983 J Infect Dis 148:93-100), including toxin-neutralizing IgA in serum and stool (Johnson, S. et al. 1995 Infect Immun 63:3166-3173). Systemic and mucosal antibody response appears to be associated with protection from subsequent infections. Disease progression and recurrence seem to be associated with the different subsets of antibodies in the circulation (Katchar, K. et al. 2007 Clin Gastroenterol Hepatol 5:707-713), and the exact reason behind this observation is unclear. A TcdA-specific antibody substantially enhanced the cytotoxic activity of TcdA on macrophages or monocytes through Fc gamma receptor I-mediated endocytosis as was shown in He, X. et al. 2009 Infect Immune 77:2294-2303, which is incorporated herein by reference hereby in its entirety.

### Vaccine Development

Antibodies specific for both toxins are needed to protect animals colonized with highly toxigenic strains (Babcock, G. J. et al. 2006 Infect Immun 74:6339-6347). The toxicity of unmodified C. difficile toxins prevents direct use as vaccines; therefore, toxoid generated by formaldehyde crosslinking or toxin fragments that lack the catalytic domain have been utilized as candidate vaccines (Ghose, C. et al. 2007 Infect Immun 75:2826-2832; Torres, J. F. et al. 1995 Infect Immun 63:4619-4627; Ward, S. J. et al. 1999 Infect Immun 67:5124-5132).

Parental toxoid immunization provides only partial protection against CDI in the acute hamster disease model and induces serum IgG responses in human volunteers (Kotloff, K. L. et al. 2001 Infect Immun 69:988-995; Torres, J. F. et al. 1995 Infect Immun 63:4619-4627). It is however unclear whether this regimen of vaccination is effective against chronic disease and provides mucosal protection. Due to the nature of the intestinal infection, a mucosal route of vaccination capable of generating systemic and mucosal antibody responses against C. difficile toxins is desirable. Consequently, mucosal routes of immunization have been tested using toxoids with the mucosal adjuvant cholera toxins (CT). A combination of intranasal and intraperitoneal immunization provided full protection from both lethal disease and diarrhea in hamsters after C. difficile oral challenge (Torres, J. F. et al. 1995 Infect Immun 63:4619-4627). Transcutaneous routes of toxoid immunization caused a mucosal IgA response with CT as adjuvant (Ghose, C. et al. 2007 Infect Immun 75:2826-2832). Chemically detoxified toxoid induces a poorer mucosal response than molecules that can target receptors on mucosal surfaces (Cropley, I. et al. 1995 Vaccine 13:1643-1648; Torres, J. F. et al. 1995 Infect Immun 63:4619-4627), since toxoid is unable to bind to the mucosal surface due to the nature of formaldehyde treatment (Kunkel, G. R. et al. 1981 Mol Cell Biochem 34:3-13). As such, strong mucosal adjuvants, such as CT or E. coli heat liable toxin (LT), are necessary for induction of mucosal immunity.

Another form of experimental vaccine for CDI is recombinant expressed toxin fragments that are devoid of GT domain therefore non-toxic. Although TcdB may be more important than TcdA in pathogenesis of the disease in CDI (Lyras, D., et al. 2009 Nature 458:1176-1179), only fragments that contain a portion of receptor binding domain of TcdA have been tested as candidate vaccine (Sauerborn, M. et al. 1997 FEMS Microbiol Lett 155:45-54; Ward, S. J. et al. 1999 Infect Immun 67:5124-5132). Recombinantly expressed toxin fragments are relatively easy to produce in large quantities. Deletion of significant parts of the holotoxin may affect the overall receptor binding and uptake of toxin fragments by the epithelium. In addition, it is possible that the deletion of a large portion of the toxins affects stereo composition of the fragment. Consequently, fragments have lost the ability to induce antibodies against the deleted portion and against stereotypically significant epitopes of the holotoxins, reducing considerably their antigenicity. Holotoxins in contrast induce antibodies specific for epitopes across the entire toxins (Babcock, G. J. et al. 2006 Infect Immun 74:6339-6347).

Intramuscular immunization with a DNA vector expressing C-terminal receptor-binding domain of TcdA was shown to induce systemic IgG response against that fragment, and the immunized mice survived from a challenge with wild type TcdA (Gardiner, D. F. et al. 2009. Vaccine 27:3598-3604). DNA vaccines have been shown to generate mucosal immune response (van Ginkel, F. W. et al. 2000 Emerg Infect Dis 6:123-132). Plasmid DNA was used in clinical trials to induce systemic antibodies and CTL against several pathogens, including hepatitis B virus, herpes simplex virus, HIV, malaria, and influenza, but failed to induce adequate responses in the mucosal compartment in these cases (van Ginkel, F. W. et al. 2000 Emerg Infect Dis 6:123-132). Both holotoxins have large sizes and are consequently predicted to have over 20 O- and N-glycosylation sites as expressed in mammalian cells rendering difficulty in expressing whole or even a large portion of the toxin genes in mammalian cells without significant alteration of the stereo composition of the protein by glycosylation. Small portions of toxin fragment expressed using a DNA reduced antigenicity.

Because options for preventing and treating CDI are rapidly diminishing, particularly against recently emerged hypervirulent C. difficile strains, novel strategies are needed. Current vaccines using toxoid, toxin fragments, or fragment-expressing DNA vectors have various disadvantages discussed above. Prior attempts to express C. difficile holotoxins have been limited (Park, E. J. et al. 1999 Exp Mol Med 31:101-107; Pizza, M. et al. 1994 J Exp Med 180:2147-2153).

These problems are addressed in Examples herein in which wild type and GT-deficient holotoxin proteins were expressed in an endotoxin-free B. megaterium system with a high expression yields. These mutant toxin proteins were found to have intact C-terminal regions and conformations, and to maintain equivalent adjuvant activity, antigenicity, and affinity to the mucosal epithelium as wild type toxins. Immunization of mice with atoxic holotoxin proteins is here observed to have induced stronger antibody response and protective immunity than did the corresponding toxoid, and induced a wider spectrum antibody response then did toxin fragment. In addition, vaccination of mice with a specifically designed chimeric protein containing elements from both TcdA and TcdB (cTxAB) induced antibodies specific for both toxins and protected mice from lethal challenge by both toxins. Therefore, each of atoxic toxin proteins (aTxAB) and the chimeric protein (eTxAB) in Examples herein are evaluated herein as vaccine candidates for safety, immunogenicity, and assessment of efficacy as administered by various routes and regimens of immunizations.

Two immunogens (aTxAB and cTxAB) were constructed and were evaluated for relative efficacy to induce robust mucosal and/or systemic protection against oral challenge by C. difficile. Several regimens of mucosal immunizations (oral, intranasal and sublingual) designed to induce protection against systemic and mucosal challenge wild type toxins were assessed for efficacy. Mucosal immunization is suitable to administer to patients who would benefit from receiving multiple boosters. The protective efficacies of the various immunization regimens analyzed herein were assessed using a mouse acute infection model. Immunization methods evaluated as efficacious by data obtained using the mouse infection studies were then evaluated in the chronic gnotobiotic piglet model of CDI. Orally infected piglets display several key characteristics observed in humans with CDI. Depending on age and infectious dose, these include symptoms of acute illness of diarrhea, anorexia and possible fatality; or chronic disease with typical pseudomembranous colitis, inflammation and profound mucosal damage, manifested with prolong intermittent diarrhea, poor health, and weight loss.

Safety and immunogenicity of two immunogens (aTxAB and cTxAB) were observed herein, indicating that these proteins would be suitable for development of an effective needle-free, temperature resistant vaccine candidate which simultaneously would protect patients against gastrointestinal and systemic manifestations of illness. Mucosal adjuvants such as mLT and CpG for intranasal and sublingual immunizations, and CT (modified cholera toxin) microencapsulation for oral immunization were examined.

### Animal Disease Models

CDI has been studied in a number of animal species, including hamsters, guinea pigs, rabbits, and germ-free mice and rats (Abrams, G. D. et al. 1980 Gut 21:493-499; Czuprynski, C. J. et al. 1983 Infect Immun 39:1368-1376; Fekety, R. et al. 1979 Rev Infect Dis 1:386-397; Knoop, F. C. 1979 Infect Immun 23:31-33). The most widely used model is the hamster, in which CDI is induced with toxigenic C. difficile infection of antibiotic treated animals. The disease in hamsters primarily affects the cecum with some involvement of the ileum; animals develop diarrhea which is fatal due to severe enterocolitis. The lethal disease in hamsters does not represent the usual course and spectrum of CDI in humans. The hamster model has been used for three decades to study therapy and mechanisms of disease. Animal models that more closely resemble human CDI have been developed (Chen, X. et al. 2008 Gastroenterology 135:1984-1992; 129), including a C57BL/6 mouse model which is susceptible to C. difficile after contact to a mixture of antibiotics for three days (Chen, X. et al. 2008 Gastroenterology 135:1984-1992). The mice developed diarrhea and lost weight. Disease severity varied from fulminant to minimal in accordance with the challenge dose. Typical histologic features of CDI were evident.

C. difficile also causes naturally occurring diarrhea-associated disease in swine, most typically during the first seven days of life (Songer, J. G. et al. 2006 Anaerobe 12:1-4; Songer, J. G. et al. 2000 Swine Health and Production 8:185-189; Songer, J. G. et al. 2005 J Vet Diagnost Investigat 17:528-536). CDI is the most common diagnosis of enteritis in neonatal pigs (Songer, J. G. et al. 2006 Anaerobe 12:1-4), perhaps, because of the similarities in the anatomy and physiology of the digestive track, nature of the diet, and the associated gut microflora which result from such combination of factors. This makes piglets a potential model for CDI. The germfree or the gnotobiotic (GB) piglet offers additional advantage in that it is a well characterized, controlled, optimized and standardized model which requires no antibiotic treatment to sterilize the gut. Challenging piglets with the hypervirulent strain 027/BI/NAP1 produced consistent results, with 100% colonization within 48 hours of inoculation, 100% morbidity, and severity of disease and mortality dependent upon dose and age at inoculation (Steele, J. et al. 2010 J Infect Diseases 201:428). Additionally, the piglet model offers a range of disease spectrum, from acute and lethal to chronic diarrhea with the characteristic pseudomembranous colitis with intensity and duration that can readily be manipulated under a controlled laboratory setting. The range of clinical signs, including systemic consequences, is similar to that observed in human cases, making the GB piglet an attractive model to perform preclinical evaluation of vaccine candidates and therapeutic agents.

Use of Animal Models to Evaluate Efficacy of the aTxAB and/or cTxAB Immunogens

The aTxAB and/or cTxAB immunogens are evaluated for efficacy in preventing the development of symptoms of diarrhea and/or systemic intoxication consistent with acute or chronic CDI, using the gnotobiotic piglet model of C. difficile infection, and the mouse CDI model.

There are no in vitro techniques available that can mimic an in vivo immune response and pathologic outcome generated as a consequence of toxin inoculation. Murine models of infection are useful models for analyzing naturally occurring host immune responses due to ease of manipulation, existence of genetically inbred strains and abundant immunological reagents. A systemic challenge model is used to evaluate the protection generated by parenteral or mucosal immunizations. The ileal loop assays include precise control of the dose of toxin inoculated into a microenvironment, and therefore dissection and definition of mucosal protection generated by a mucosal route of immunization. Traditionally, a hamster is widely used animal model of CDI. This model has been used extensively for CDI studies, and hamsters are extremely sensitive to the infection. Mortality often approaches 100% within 48 hours of infection with virulent strains, and a fatal disease may develop from just one colony forming unit cfu (Keel, M. K. et al. 2006 Vet Pathol 43:225-240). Acute onset in hamsters leaves little time for investigation of events in pathogenesis compared to disease in humans. For these reasons, investigators actively seek animal model that closely resembles human causes of the diseases. The mouse infection model closely resembles human course of the disease (Chen, X. et al. 2008 Gastroenterology 135:1984-1992). Use of the two animal models allows evaluation of safety of candidate vaccines essential prior to application to control or treatment of CDI in humans. Both the mouse and the piglet CDI models were used in Examples herein to generate preclinical vaccine evaluation data required for PDP leading to Phase I clinical trials in human volunteers.

The efficacy of the top ranked regimens of systemic and/or mucosal immunizations is assessed for protection of animals against acute and chronic CDI induced by orally challenged animal models with C. difficile. Considering the complex of CDI manifestations (from mild diarrhea, pseudomembranous colitis, to fulminant disease and recurrence and multiple relapses), two animal CDI models are needed to fully evaluate the efficacy of immunogens with a view to perform preclinical evaluation on one of them as a potential candidate vaccine. Both mouse and gnotobiotic (GB) piglet models are used to evaluate whether an immunization, based on the above findings, is capable of preventing animals orally challenged with C. difficile from developing CDI. In addition, assessment vaccination reduction or elimination of C. difficile-mediated, ongoing, chronic diseases in the piglet infection model is performed. The persistence and magnitude of antitoxin antibodies are measured over several months and assessment is made of necessity of at least one additional booster to prevent recurrence or relapse of CDI in piglets.

Efficacy of the top ranked regimens of systemic and/or mucosal immunizations is initially examined for ability to protect mice against acute CDI induced by oral challenge with C. difficile followed by examining the efficacy of selected regimens in the piglet model of chronic CDI.

In piglet model, two groups of piglets are maintained for additional three months after the oral challenge with C. difficile, to monitor the levels of specific serum and secretory antibody, after which they are challenged with C. difficile a second time to assess protection against recurrence/relapse; if the specific antibody levels are deemed low, a booster immunization is given before the second challenge. The efficacy of such vaccines is evaluated using symptomatic, pathological and immunological parameters established for the piglet model. Comprehensive preclinical evaluation of efficacy of candidates is performed using aTxAB or cTxAB, using various routes with or without adjuvant, as the basis for a vaccine. The best candidate vaccine is selected for clinical evaluation. The examples herein show data obtained describing duration of protection against relapses, and the benefit of booster immunizations.

### Treatment Design for Mice

Absense of toxicity of the mutant holotoxins and cTxAB are determined by assay of cytotoxicity and in vivo toxicity in mice challenged systemically.

Immunizations are carried by intraperitoneal (IP) injection of 5 or 10 μg of purified antigens with alum as adjuvant. Antibody titers are measured by standard ELISA. Serum neutralizing titers are measured by blocking cytotoxicity of wild type toxins on mouse intestinal epithelial line CT26 cells. To evaluate the protection of vaccination against systemic toxins, the immunized mice are challenged IP with lethal doses of either wild type TcdA or TcdB, and mouse disease and mortality are monitored.

To evaluate the protective immunity against CDI, immunized mice are orally challenged with C. difficile vegetative cells or spores and development of symptoms such as diarrhea, weight loss, and mouse survival is monitored. Intestinal inflammation and tissue damage is assessed by histopathology analysis.

To evaluate the protection by cTxAB vaccination for recurrent CDI, immunized mice are treated with antibiotic cocktail and then orally rechallenged with C. difficile spores 30-day after initial C. difficile challenge. Mouse survival and symptoms of the disease are monitored.

Mice are treated in groups of 10 C57BL/6 or Balb/c mice, aged 6-8 weeks. Each set of treatments includes a positive control (toxoid) and a negative control (vehicle plus adjuvant where appropriate). Animals are vaccinated three times at two weekly intervals, and one week after the last immunization mice are further challenged with the relevant wild type toxin. Mice are treated with antibiotics after the last immunization and before oral challenge with laboratory strain VPI 10463, the hypervirulent C. difficile strain 027, or with the control (tcdA⁻ tcdB⁻ avirulent) strain CD37. Before each immunization or challenge with toxin or bacteria, sera and fecal samples are collected and analyzed for specific antibody isotypes by ELISA and by the cell cytotoxicity assay. After challenge with toxin or bacteria mice are monitored closely for symptoms of illness which include reluctance to move, anorexia, arched back, lethargy, loss of body weight, pasty stools, ruffled coat, and recumbency. Seriously sick animals are euthanized. In animals challenged orally with C. difficile, bacterial excretion in feces is quantified, and visceral organs are formalin-fixed and examined histologically for abnormalities as shown in Examples herein. The presence of toxins circulating in blood (animals challenged with toxins), or in blood and in feces (animals challenged orally with C. difficile), is quantified, using the ultrasensitive assay as described (He, X. et al. 2009 J Microbiol Methods 78:97-100, incorporated herein by reference in its entirety).

Balb/c or C57BL/c mice were treated with antibiotic cocktail (a mixture of kanamycin, gentamicin, colistin, metronidazole, and vancomycin) followed with oral inoculation of C. difficile as described previously (Chen, X et al. 2008 Gastroenterology 135: 1984). Ten days after the third immunization, mice were given 10⁵ CFU of vegetative bacteria (laboratory VPI10463 strain) using gavage. To assess long-term immunity, mice were orally challenged with 10⁶ CFU of vegetative bacteria three months after the third immunization. In some Examples, the immunized mice were challenged with 10⁶ spores of UK1 (027/B1/NAP1 strain, VA Chicago Health Care System). To induce relapse CDI, surviving mice were given antibiotic cocktail treatment followed with an oral C. difficile spore (10⁶/mouse) inoculation 30 day-post the primary infection. The secondary challenge induces a similar clinic manifestation and intestinal histopathology as the primary CDI. The recurrent disease and death were monitored.

Toxin Shedding after C. difficile Challenge

After primary and secondary challenge with C. difficile spores, mouse feces were collected and dispersed in an equal volume (w/v) of PBS containing protease cocktail, and the supernatants were collected by centrifugation and stored at −80° C. until use. To measure toxin-mediated cytotoxicity of fecal samples, the supernatants were diluted (final 100×) and filtered before adding to CT26 cell monolayers. Cell rounding was observed using a phase-contrast microscope. Goat anti-TcdA and -TcdB polysera (Techlab Inc., Blacksburg, Va.) were used to determine the specific activity of the C. difficile toxins.

### Cytokine Measurement

Cytokine concentration is determined in feces three times per week, and at necropsy from the large intestinal contents for IL-1β, IL-4, IL-6, IL-8, IL-10, IL-12, TNF-α, TGF-β, and IFN-γ using commercially available porcine cytokine ELISA kits (Invitrogen and R&D). Samples are stored at −20° C. until use. Fecal samples and large intestinal contents are diluted 1:2 to 1:10 with sterile PBS, depending on the consistency of the sample, thoroughly mixed using a vortex, then centrifuged, and the supernatant is added to reagent wells in the assay. The assay is performed following the manufacturer's instructions, and cytokine concentration is determined based on the standard curve (Steele, J. et al. 2010 J Infect Diseases 201:428).

### Antibody Titers and In Vitro Neutralizing Assay

The neutralizing titers against TcdA and TcdB for sera, intestinal lavage fluid, and fecal samples are determined. One week after the last immunization with the optimal dose of aTxAB, cTxAB, or toxoids, serum from each immunized mouse is collected. Sera from each group are pooled and neutralization of the cytotoxicity of either TcdA or TcdB is measured. Neutralizing titers and the optimal doses of the immunogens for parenteral immunization are determined. The calculated LD₅₀ᵢ toxin challenge doses are used to determine the level of protection induced by each immunogen. The protection level correlates with serum neutralizing titers, and, both aTxAB and cTxAB have significantly higher LD₅₀ᵢ and neutralizing titers than those of the toxoid. The LD₅₀ᵢ and neutralizing titers are used as references.

In mouse model, one day before an immunization and seven days after a previous immunization, serum samples from each immunized mouse were collected and IgG titers were measured using standard ELISA against purified each native or recombinant wild type holotoxins. In some treatments, the IgG titers were compared by using native toxins to coat ELISA plate with those using our recombinant toxins, and the results were essentially the same, showing that the antibody titers against His(6)-tag were negligible. In some treatments, serum antitoxin IgM and IgA, and fecal IgG and IgA were assessed by ELISA. To assess in vitro neutralizing activities of the serum samples, mouse intestinal epithelial cell line CT26 sensitive to both TcdA and TcdB was used. The neutralizing titer is defined as the reciprocal of the maximum dilution of serum that fails to block cell rounding induced by a standard concentration of a toxin. This concentration is four times the minimum dose of the toxin that causes essentially all CT26 cells to round after a 24-hour contact to the toxin. Wild type TcdA at 1.25 ng/ml or TcdB at 0.0625 ng/ml causes 100% of CT26 cells rounding after 24 hours of toxin treatment. Therefore, TcdA at 6 ng/ml or TcdB at 0.25 ng/ml was mixed with each of serially diluted serum samples which were then applied to CT26 cells. Cell rounding was observed using a phase-contrast microscope after 24 hours of incubation.

### Ultrasensitive Immunocytoxicity Assay

The current available assays to diagnose CDI, such as cytotoxin B assay, antibody-based immunoassays, GDH assay etc., have serious limitations. An ultrasensitive, tissue culture-based assay was developed based on recent findings and is here referred to as the immunocytotoxicity assay (He, X. et al. 2009a Infect Immun 77:2294-2303; He, X. et al. 2009b J Microbiol Methods 78:97-100; Herrmann et al., International patent application publication number WO 2010/006326 published Jan. 14, 2010 incorporated herein by reference hereby in its entirety). This assay detects the presence of less than 1 pg/ml of toxin in biological samples within four hours (He, X. et al. 2009 J Microbiol Methods 78:97-100, incorporated herein by reference). This assay was used to assess the systemic toxins in acute mouse and piglet models and the effect of antitoxins to reduce or eliminate the toxins.

The efficacy of parenteral immunization of the candidate vaccines (aTxAB and cTxAB) is evaluated and the optimal doses of immunization are determined to induce maximum antibodies to induce a protective response.

### Dose Optimization

The initial treatment used 5 μg total proteins per injection of the immunogens. Dose optimization of aTxAB and cTxAB and toxoids was determined by determining the results of using doubled and halved optimized doses for parenteral immunization. If an adjuvant is used, e.g., mLT, the same amount of the adjuvant is mixed together with the immunogen before injection. For each dose and route of immunization, both systemic and mucosal IgG and IgA responses were monitored and neutralizing titers were measured. The lowest amount of antigen required to induce the highest level of serum and/or mucosal antibody response for each immunogen was established.

### Establishment of Challenge Dose of LD₅₀ᵢ

One week after the third immunization with the optimal dose of aTxAB, cTxAB, or toxoids, mice were challenged with doubling doses of LD₅₀ₙ, designated as doses causing death of 50% of naïve mice by wild type toxins. The dose that causes death of 50% of immunized mice were determined and designated as LD₅₀ᵢ. The LD₅₀ᵢ of each toxin for each immunogen was determined and the LD₅₀ᵢ of aTxAB were similar to that of cTxAB, both of which were significantly higher than those of toxoids.

### Mice, Cell Lines, and Toxins

Six- to 12-week-old BALB/c, CD1 and C57BL/6 mice were purchased from Jackson Laboratory (Bar Harbor, Me.) and housed in dedicated pathogen-free facilities. The mice were handled and cared for according to Institutional Animal Care and Use Committee (IACUC) guideline under protocols G950-07, G889-07, and G795-06. For evaluating systemic vaccination, ten mice per group (total four groups) were used and five mice were used for IP challenge by each toxin or toxin combination with two routes of immunization and three replicates of each treatment, with safety evaluation.

The murine colonic epithelial cell line CT26, the human colon epithelial cell lines HT-29 and HCT-8, and the monkey kidney cell line Vero were obtained from American Type Culture Collection (ATCC; Rockwille, Mich.). Cells were maintained in Dulbecco's modified Eagle medium containing 10% fetal bovine serum, 100 U/ml penicillin, 100 μg/ml streptomycin, 2 mM L-glutamine and 1 mM sodium pyruvate. Native TcdA and TcdB toxins were purified from culture supernatants of toxigenic C. difficile strain VPI 10463 as previously described (Yang, G et al. 2008 BMC Microbiol 8: 192, incorporated by reference herein). Full-length wild type recombinant TcdA and TcdB proteins were purified from total crude extract of Bacillus megaterium as described previously (Yang, G et al. 2008 BMC Microbiol 8: 192). The biological activity of recombinant holotoxins was identical to their native forms (Yang, G et al. 2008 BMC Microbiol 8: 192). The highly purified recombinant toxins appeared as a single band on an SDS-PAGE gel and were devoid of detectable TLR2 and TLR4 ligand activity as determined by bioassays (He, X et al. 2009a. Infect Immun 77: 2294; Sun, X et al. 2009 Microb Pathog 46: 298, each of which is incorporated hereby in its entirety herein) and were used in Examples herein, unless otherwise specified.

Balb/C or C57BL/6 mice were immunized intraperitoneally (IP) with 5 μg of purified mutant toxins in PBS with alum as adjuvant for each injection. Control mice were injected PBS with alum. Using aTcdA mixed with aTcdB (5 μg each) or cTxAB as immunogens, a total of 10 μg of protein per injection was administered, and mice were given three immunizations at 10 to 14 day intervals.

Systemic toxin challenge: Balb/c mice (four to six week old) were IP injected with wild type TcdA or TcdB (100 ng/mouse), aTcdA (100 μg), or aTcdB (100 μg/ml). Mice were observed closely for signs of disease and euthanized when they became moribund.

### Assessment of Binding of Immunogens to Mucosal Epithelium

A challenge facing mucosal antigen delivery is inefficient uptake of antigens by the mucosa. Although the receptor(s) are undefined, the receptor binding domain (RBD) of both C. difficile toxins contains multiple cell-wall binding repeats with high affinity to epithelial cells. Both aTxAB and cTxAB contain intact RBDs of C. difficile toxins, thus it is most likely that both immunogens can bind to epithelial cells with high affinity. To assess the binding of aTxAB and cTxAB to epithelial cells, the proteins are biotinylated before administration to mice using the method for biotinylation described by Keel and Songer (Keel, M. K. et al. 2007 Vet Pathol 44:814-822), in which neither the activities of the toxins, nor their binding to epithelium is affected. Six hours after mucosal (IN, SL, and oral) administration of the biotinylated aTxAB or cTxAB, mice are sacrificed and tissue sections are prepared for immunohistochemistry staining. To harvest sublingual mucosa, the floor of the mouth together with the tongue is excised en bloc from the mandible with thin curved scissors. The nasal mucosa is dissected following the method described in detail by Eriksson et al (Eriksson, A. M. et al. 2004 J Immunol 173:3310-3319). To assess the binding of the immunogens to GI track after orally administration, the antral regions of the stomach and segments of the intestine from the immunized mice are collected. Specimens are fixed with paraformaldehyde and embedded in paraffin. For immunohistochemistry staining, the deparaffinized 6-μm-thick sections are pre-treated with biotin blocking kit before stained with HRP-conjugated avidin as described in Examples herein.

### Serum and Mucosal (Intestinal and Fecal) Antibody Response

The serum antibody response is analyzed as described in Examples herein. To examine the mucosal antibody response, intestinal lavage fluid (IL) is collected and fecal samples from mice and toxin-specific IgG and IgA are measured.

One day prior to each immunization and one week after the last immunization, fecal and IL samples are collected (Elson, C. O. et al. 1984 J Immunol Methods 67:101-108). Briefly, each mouse is kept on a 15 cm×15 cm wire mesh placed on top of a plastic petri dish containing 1 ml of a protease inhibitor cocktail. The mouse is restrained in a glass beaker on top of the wire mesh. To induce discharge of intestinal contents, four doses of 0.5 ml of lavage solution (25 mM NaCl, 40 mM Na₂SO₄, 10 mM KCl, 20 mM NaHCO₃, and 48.5 mM (162 g/l) polyethylene glycol (PEG) (average M_{w} 3350) are given at 15-minutes intervals using gavage. Thirty minutes after the last dose of lavage solution, the mice are given 0.1 mg of pilocarpine intraperitoneally. Intestinal contents (up to 0.5 ml) discharged over the next 20 minutes are collected in plastic tubes and kept frozen at −70° C. until use. Immediately before initiating the intestinal lavage procedure, two pieces of freshly voided feces are collected into 1.5-ml pre-weighed micro-centrifuge tubes. The feces are weighed before adding two volumes of PBS with protease inhibitor cocktail. Solid matter is suspended by extensive vortexing followed by centrifugation at 16,000×g for 10 minutes and the clear supernatants are stored at −70° C. until assayed.

Titers of antibodies specific for TcdA, TcdB IgG and IgA are determined by ELISA. Purified native TcdA or TcdB are used to coat the plates, which allows to minimize the cross reaction to His₆ Tag or possible contaminants in the immunogens. The detection limits of antitoxin IgA or IgG were set as two times of OD405 over background in the well with the lowest dilution.

### Histopathological Analysis

Histopathological analysis was performed to evaluate mucosal damage and inflammation induced by the toxins. Resected colon or cecum tissues were fixed in 4% formaldehyde buffered with PBS and then embedded with paraffin. De-paraffinized 6-μm-thick sections were stained with hematoxylin and eosin (H&E) for histological analysis.

Protection Against Mucosal Challenge with the Toxins

Rabbit antisera specific for TcdA were observd to block TcdA-induced intestinal inflammation and tissue damage as was shown in mouse ileal loop model. Mucosal IgA and IgG antibodies against toxins are generated and ability to protect mice against toxin-mediated destruction of the mucosa is examined. Because pure TcdB has no enterotoxicity and does not induce mucosal inflammation and tissue destruction in mice, only mucosal protection against TcdA is examined using ileal loop model. The ability of mucosal immunization of aTxAB or cTxAB to induce mucosal protection against TcdB and against TcdA in orally challenged C. difficile mouse and piglet infection models is also examined.

The ileal loop models are used one week after the third immunization. In normal Balb/c mice, a high dose of 50 μg of wild type TcdA was observed to cause substantial fluid accumulation and mucosal damage, whereas a lower dose of 10 μg of TcdA caused only mild mucosal destruction within four hours of toxin treatment. Therefore, these two doses are used. Three 3-cm loops are ligated in each mouse and injected with 10 or 50 μg of wild TcdA, or an equal volume of PBS (100 μl). The same treatments are performed in control placebo treated mice. The toxin-induced fluid accumulation is quantitated, and data are analyzed using one-way ANOVA. P values between groups are determined using Bonferoni's multiple comparison test.

In addition to assessing the fluid accumulation, the pathological signs, such as neutrophil infiltration and villus damage, are evaluated histologically and compared between the groups. Histopathological and neutrophil myeloperoxidase (MPO) activity assays are performed to evaluate mucosal damage and neutrophil infiltration. The loops are collected, and the resected intestines are fixed in 4% formaldehyde buffered with PBS and then embedded with paraffin. Deparaffinized 6-μm-thick sections are stained with haematoxylin and eosin (H&E) for histological analysis, and the tissue injuries are blindly scored by a histologist. Histological grading criteria used are as follows: 0, minimal infiltration of lymphocytes, plasma cells, and eosinophils; 1+, mild infiltration of lymphocytes, plasma cells, neutrophils, and eosinophils plus mild congestion of the mucosa with or without hyperplasia of gut-associated lymphoid tissue; 2+, moderate infiltrations of mixed inflammatory cells, moderate congestion and edema of the lamina propria, with or without goblet cell hyperplasia, individual surface cell necrosis or vacuolization, and crypt dilatation; 3+, severe inflammation, congestion, edema, and hemorrhage in the mucosa, surface cell necrosis, or degeneration with erosions or ulcers (Tones, J. F. et al. 1995 Infect Immun 63:4619-4627). To measure MPO activity in the samples, a portion of the resected ileum is freeze-dried and homogenized in 1 ml of 50 mM potassium phosphate buffer with 0.5% hexadecyl trimethyl ammonium bromide and 5 mM EDTA. The tissues are disrupted with sonication and freeze-thaw cycles, and centriguged. MPO activity in the supernatant is determined using substrate o-phenylenediamine in 0.05% of H₂O₂, and absorbance is measured at 490 nm using a plate reader.

Mucosal vaccination is expected to protect against TcdA challenge in the intestine. TNF-α was observed to play a crucial role in C. difficile toxin-induced intestinal inflammation. TcdA induced a complete destruction of villi and massive infiltration of immune cells in wild type mice, and TNFR KO mice showed mild damage of intestinal villi and moderate infiltration of immune cells in response to TcdA.

### Statistical Analysis of Piglet Model

In piglet model, the data obtained from treatments are analyzed using a non-parametric test (Wilcoxon analysis) following ANOVA using SigmaStat v. 3.1 (Systat Software, Inc.). For four groups, including a control group, for a power of 0.8 and alpha=0.05, a sample size of 5-118 is required depending on level of T² desired. Seven animals/group (n=7) were used for challenge studies involving evaluation of vaccine candidates. Survival curves are compared and analyzed by Log-rank (Mantel-Cox) Test or Gehan-Breslow-Wilcoxon Test using GraphPad Prism software.

These data are complemented with Group Pair-Wise Comparisons (Levene's/ANOVA-Dunnett's/Welch's). The Levene's test is used to assess homogeneity of group variances for each specified endpoint and for all collection intervals. If Levene's test is not significant (p>0.01), a pooled estimate of the variance (Mean Square Error or MSE) is computed from a one-way analysis of variance (ANOVA) and utilized by a Dunnett's comparison of each treatment group with the two control groups. If Levene's test is significant (p<0.01), comparisons with the control group are made using Welch's t-test with a Bonferroni correction. Results of pair-wise comparisons are reported at the 0.05 and 0.01 significance levels. Endpoints are analyzed using two-tailed tests unless indicated otherwise.

### Technological Advantages

Both systemic and mucosal immunity provide protection against enteric pathogens and pathogenic products such as toxins (Byrd, W. et al 2006 FEMS Immunol Med Microbiol 46:262-268; Huang, C. F. et al. 2008 J Pediatr Gastroenterol Nutr 46:262-271; Lucas, M. E. et al. 2005 N Engl J Med 352:757-767; Perez, J. L. et al. 2009 Vaccine 27:205-212). Because TcdA and TcdB are virulent factors for C. difficile, an antitoxin antibody preparation can convey full protection from oral C. difficile challenge in animals (Kink, J. A. et al. 1998 Infect Immun 66:2018-2025; Lyerly, D. M. et al. 1991 Infect Immun 59:2215-2218). Antibodies against both toxins, but not against TcdA or TcdB alone, protect toxigenic C. difficile infection in hamster model (Fernie, D. S. et al. 1983 Dev Biol Stand 53:325-332; Kim, P. H. et al. 1987 Infect Immun 55:2984-2992; Libby, J. M. et al. 1982 Infect Immun 36:822-829). An evaluation of the routes of delivery of toxoid vaccine in hamsters assessing protection from both lethal disease and diarrhea have found that a combination of mucosal and parental immunization provided complete protection from death and diarrhea, suggesting that induction of both systemic and mucosal immunity was necessary for optimal protection (Tones, J. F. et al. 1995 Infect Immun 63:4619-4627).

In humans, a higher level of antitoxins in serum is associated with less severe disease and less frequent relapse (Kyne, L. et al. 2000 N Engl J Med 342:390-397). Following symptomatic infection, most individuals develop anti-TcdA and anti-TcdB antibodies in serum (Aronsson, B. et al. 1985 Infection 13:97-101; Viscidi, R. et al. 1983 J Infect Dis 148:93-100), including toxin-neutralizing IgA in serum as well as in stool (Johnson, S. et al. 1995 Infect Immun 63:3166-3173), and this systemic and mucosal antibody response appears to be associated with protection from subsequent infection.

Clostridium difficile-associated diarrhea and enteric inflammatory diseases are caused primarily by two secretory toxins. A vaccine (mucosal and/or parenteral delivery) is proposed herein to reduce the incidence and severity of Clostridium difficile infection (CDI), using recently expressed atoxic C. difficile toxin proteins in an endotoxin-free Bacillus megaterium system. The technology is an extension of a previously demonstrated successful methodology to use a B. megaterium expression system to manufacture full-length, biologically active, recombinant holotoxins, rTcdA and rTcdB (Yang, G. et al. 2008 BMC Microbiol 8:192, incorporated herein by reference). The resulting rTcdA and rTcdB were found to be found to be similar to their native counterparts after extensive examination including measurement of molecular mass and biological activity (Yang, G. et al. 2008 BMC Microbiol 8:192, incorporated herein by reference). The B. megaterium expression system has been in use for more than 50 years and has several advantages over other systems.

Two candidate vaccines are here evaluated: a mixture of atoxic full-length C. difficile toxin A and B generated by point mutations (designated as aTxAB), and a well-designed chimeric protein containing otherwise full-length TcdB but its receptor binding domain replaced to that of TcdA (designated as cTxAB). cTxAB has a small deletion (97 amino acids) in transmembrane domain thus non-toxic.

Protection against CDI has been shown to be mediated through systemic and mucosal antibodies against the two key toxins, although other virulence attributes are known to exist which may also contribute to the manifestation of CDI.

The focus was on designing a vaccine that targets both TcdA and TcdB, in order to elicit strong systemic and mucosal immunity. The aTxAB or cTxAB were found to be superior to toxoid or fragments thereof. Without being limited to any theory or mode of action the Examples herein showed that the full-length proteins which mimic the native form with correct folding of such large molecules are useful for generating a full spectrum of neutralizing antibodies. Unlike chemical-detoxified toxoid, or fragments that contain a small portion of TcdA, these atoxic holotoxins generated by point mutations maintain the same adjuvant activity, antigenicity, and affinity to mucosal epithelium as do native toxins, thus induce superior protective immunity than toxoid and wider spectrum of antibodies than fragments.

Atoxic TcdB vaccination was shown to induce antibody responses against a wide-spectrum of epitopes and potent protective immunity superior to toxoid; cTxAB immunization induced antibody and protective responses against the toxins. Furthermore, immunization of aTcdB induces rapid IgG response. People at high risk of C. difficile infection, such as under antibiotic treatment and/or hospitalization, are logical targets for prophylactic vaccination. A vaccine capable of inducing rapid protective immunity is highly desirable especially in hospitalized patients. aTcdB vaccination is capable of inducing rapid antibody response. Immunization of mice with aTcdB was shown to generate a potent IgG response after the second immunization, whereas toxoid immunization generated a detectable IgG response only after the third immunization (on day 28 post priming). The chimeric cTxAB vaccination induces potent protection in mice against lethal challenge with both TcdA and TcdB.

The ability of these atoxic recombinant proteins was eveluated to induce protective antibody responses following parenteral immunization followed by challenge with wild type toxins, followed by the evaluation of several regimens of mucosal immunizations (oral, intranasal and sublingual) designed to induce protection against systemic and mucosal challenges with wild type toxins. The protective efficacy of the various immunization regimens developed was tested in the mouse acute infection model, and the most efficient immunization method resulting from the mouse infection studies undergo preclinical evaluation in the chronic piglet model of CDI.

These Examples show that a novel C. difficile candidate vaccine was developed that is productively expressed in a safe, environmental, and endotoxin-free bacterial host, B. megaterium (Vary P S et al. 2007 Applied microbiology and biotechnology 76: 957; Yang, G et al. 2008 BMC Microbiol 8: 192). Compared to native toxins purified from C. difficile culture, the recombinant cTxAB is significantly easier and cheaper to purify in a large quantity. It is a single antigen maintaining a toxin-like conformation and capable of inducing potent neutralizing antibodies against the both toxins. This candidate vaccine not only induces full and long-lasting protection against C. difficile-associated morbidity and mortality, but also rapid protection against primary and recurrent CDI. Examples herein show that both primary and recurrent CDI can be prevented by systemic antibodies through parenteral vaccination.

### EXAMPLES

### Example 1

### Protection Against CDI in Mouse Model

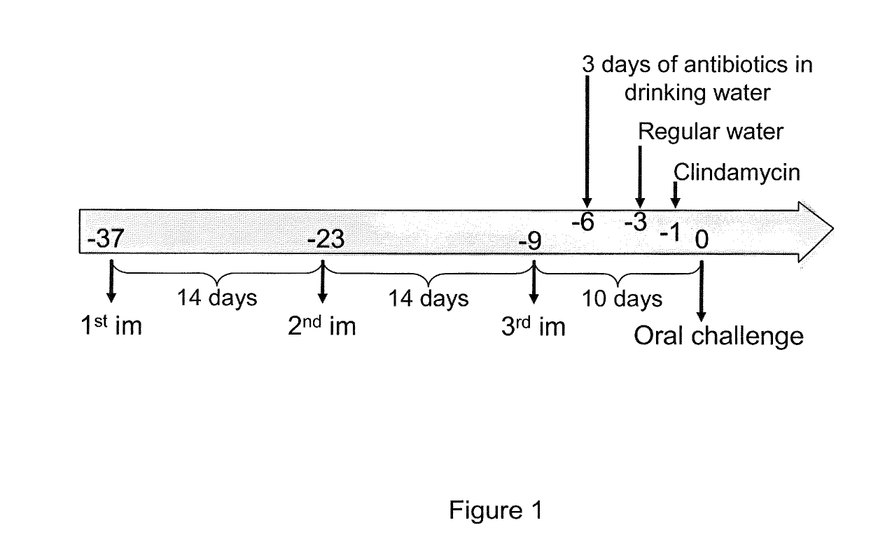

The mouse CDI model was established following the methods described by Chen. This model is used herein to determine whether immunization of mice with the candidate vaccines induces protective immunity against C. difficile infection. The immunization and challenge scheme is shown in FIG. 1.

After antibiotic treatment, more than 90% of wild type naïve mice became moribund after 10⁵ CFU of C. difficile challenge, and 10⁴ CFU of C. difficile challenge leads to less than 50% of mice death. Immunization with the immunogens protects mice against either 10⁵ or 10⁴ CFU of C. difficile challenge, and none of the mice should exhibit any sign of disease or weight loss. In addition, the amount of bacteria for the challenge dose is lowered and the protection against more chronic-like disease induced by the low dose of bacteria is examined. In all these cases, the biological activity of secreted toxins in feces is measured using the ultrasensitive immunocytotoxicity assay (He, X. et al. 2009 J Microbiol Methods 78:97-100, incorporated herein by reference in its entirety).

The immunized mice are fully protected from either 10⁵ or 10⁴ CFU of C. difficile challenge. The surviving mice are kept for monitoring anti-toxin antibody titers for up to three months. Serum samples are collected every half month and antitoxin IgG and IgA antibody titers are measured. After three months, mice are treated with antibiotics followed with oral 10⁵ CFU C. difficile challenge. The correlation between the persistence of anti-toxins antibodies and protection against bacterial rechallenge is established. The aTcdB immunization is expected to induce long-lasting protective immunity. The immunized mice are expected to be fully protected from lethal TcdB challenge. Therefore, mice immunized with aTxAB or cTxAB under optimized regimens is expected to induce long-lasting protective immunity. This is important given the fact that CDI patients often suffer relapses. If the protective immunity is not long-lasting, additional boosts are administered. After immunization and antibiotic treatment, groups of mice (5 per group) are orally challenged with escalating doses of C. difficile bacteria, starting from 10⁵ CFU. The susceptibilities of mice that are immunized with an immunogen under optimized regimens allow to assess the efficacy of protection induced by particular routes of immunization, with or without inclusion of adjuvant.

### Example 2

### Statistical Analysis

Data collected from treatments of subjects herein were analyzed by Kaplan-Meier survival analysis, analysis of variance, and by t test or one-way ANOVA using the Prism statistical software program. Results were expressed as mean±standard error of mean unless otherwise indicated.

### Example 3

### Bacillus megaterium Expression System and Production of Recombinant Holotoxins

Due to the large size and poor stability of the proteins, recombinant C. difficile holotoxins have been difficult to produce. Bacillus megaterium, a Gram-positive, aerobic spore-forming bacterium found in widely diverse habitats from soil to fish and dried food has been industrially employed for more than 50 years because of high capacity for exoenzyme production. It is a desirable cloning host for production of recombinant proteins, and genetic tools are available with shuttle vectors carrying strong inducible promoters and affinity tags. Advantages of B. megaterium expression system compared to E. coli system include lack of alkaline proteases and stably maintaining plasmid vectors, lack of endotoxin LPS, and ability to secrete expressed heterologous protein into the medium (Malten, M. et al. 2006 Applied and environmental microbiology 72:1677-1679; Vary, P. S. et al. 2007 Appl Microbiol Biotechnol 76:957-967), making B. megaterium an attractive system to express the full-length and bioactive recombinant TcdA and TcdB proteins.

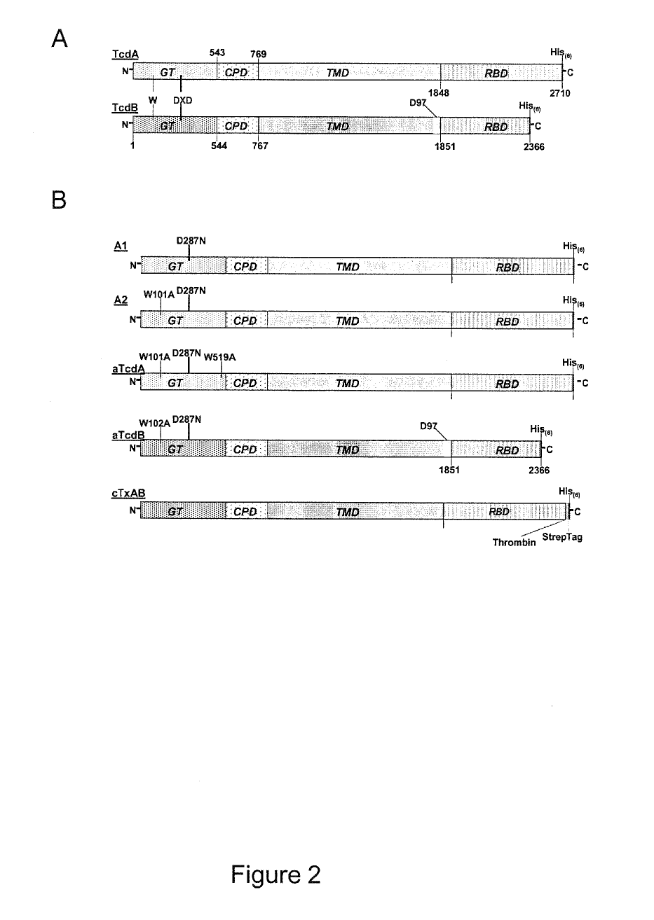

Full-length recombinant TcdA and TcdB were cloned in. B. megaterium system was cloned with a expression level reaching 10 mg/L of the toxin proteins (Yang, G. et al. 2008 BMC Microbiology 8:192, incorporated herein by reference in its entirety). A drawing of recombinant TcdA and TcdB, expressed from the first amino acid of the toxins to which a 6-amino acid His tag is attached at the C-terminus to facilitate purification is shown in FIG. 2 panel A. The biological activities of the recombinant holotoxins were observed to be identical to the native counterparts, as determined by cytotoxicity assays, glucosylation of Rho GTPases, and disruption of tight junctions of epithelial cells (Yang, G. et al. 2008 BMC Microbiology 8:192, incorporated herein by reference in its entirety). It was observed thar rTcdA, and not rTcdB, induced a dose-dependent fluid accumulation in a ligated mouse ileal loop and histological alterations were much like those described by Cavalcante et al (Cavalcante, I. C. et al. 2006 Infect Immun 74:2606-2612).

### Example 4

### Generation of Mutant Holotoxins and Chimeric Proteins

Toxoids generated by formalin-inactivation of native toxins are at present the only C. difficile candidate vaccines in clinical trials (Sougioultzis, S et al 2005 Gastroenterology 128: 764). Because TcdA and TcdB are large clostridial toxins with complex structure and conformation (Pruitt R N et al. 2010 Proc Natl. Acad. Sci. USA 107: 13467; Jank T et al. 2008 Trends in microbiology 16: 222), formalin crosslinking likely alters conformational epitopes and reduces immunogenicity. Holotoxins were generated with two or three point mutations in those amino acids of TcdA and TcdB associated with substrate binding of glucosyltransferase (Jank, T et al. 2007 J Biol Chem 282: 35222).

Because the GT domain of the toxins is associated with the toxicity of both TcdA and TcdB, holotoxins deficient in GT activity were used to analyze host immune response to the toxins and pathogenesis of the disease. GT-deficient holotoxins were generated by point mutation of key amino acids known to play a role in the substrate binding. Two point mutations (W102A and D287N) in TcdB (designed as aTcdB, FIG. 2 panel B) reduced the GT activity by up to 5 logs.

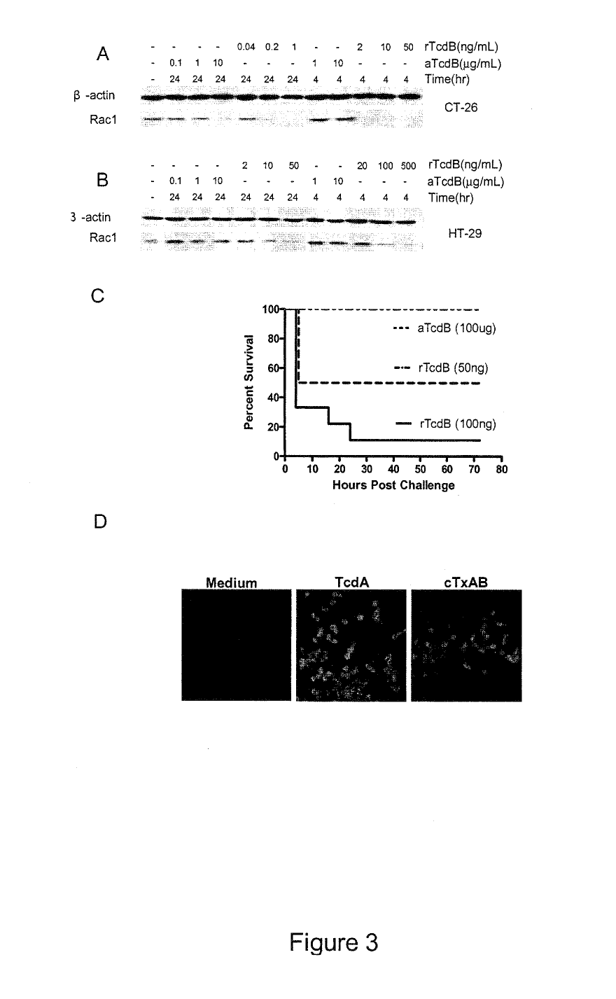

Even a very high 10 μg/ml dose of aTcdB induces only partial glucosylation of Rac1 in highly sensitive CT26 cells after 24 hour treatment (FIG. 3 panel A). In contrast, wild type TcdB induces a complete glucosylation of the Rho GTPase protein with a dose of 1 ng/ml. The aTcdB at 10 μg/ml shows no activity on the less sensitive HT29 cells (FIG. 3 panel B). Importantly, aTcdB has lost its toxicity as measured by in vitro cytotoxicity assays and in vivo mouse challenge treatments (FIG. 3 panel C). Injection of 100 ng of wild type TcdB to mice resulted in sepsis-like symptom within four hours and more than 90% of mice died within 24 hours, whereas none of the mice developed diseases or became moribund after IP injection of a very high dose (100 μg) of aTcdB (FIG. 3 panel C). Mutations at the similar conserved amino acids in TcdA also substantially reduced GT activity of TcdA. To ensure a complete loss of toxicity, an additional mutation (W519A, FIG. 2 panel B) was introduced at a conserved amino acid that is also important for substrate binding (Jank, T. et al. 2007 J Biol Chem 282:35222-3523) in TcdA, which is designated as aTcdA (triple mutations in GT domain). By utilizing the GT-mutated TcdA, the role of GT activity was demonstrated in toxin-mediated TNF-α production in macrophages (Sun, X. et al. 2009 Microb Pathg 46:298-305).

The C-terminus of TMD of TcdB (approximate 100 amino acids, designated as D97) is required for the cytotoxicity of TcdB. The recombinant TcdB in which D97 was deleted lost its toxicity on cultured cells.

Because the receptor binding domain (RBD) of TcdA possesses strong adjuvant activities (Castagliuolo, I. et al. 2004 Infect Immun 72:2827-2836; Yeh, C. Y. et al. 2008 Infect Immun 76:1170-1178), the RBD of TcdB with RBD of TcdA were replaced, creating a chimeric protein (designated as cTxAB, FIG. 2 panel B). cTxAB does not contain D97, thus for this reason is nontoxic to cultured cells and to animals. It binds cultured cells as effectively as wild type TcdA (FIG. 3 panel D), as these proteins bothcontain receptor binding domain. The single protein cTxAB contains immunodominant and immunostimulatory domains of TcdA (RBD), and most features of TcdB, therefore it is a likely effective vaccine candidate to evaluate.

To facilitate purification to test a purified version, an additional affinity tag (an 8-amino acid Streptag) was installed upstream of His₆ tag (FIG. 2 panel B). A thrombin protease cleavage site was installed (FIG. 2 panel B) to allow removal of both Streptag and His₆ tags as needed, which results in a chimeric protein containing only amino acid sequences from C. difficile toxins.

C. difficile mutant holotoxins (aTcdA and aTcdB) were generated using wild type recombinant toxins (Yang, G et al. 2008 BMC Microbiol 8: 192, incorporated herein by reference in its entirety).

Mutant GT domain genes containing point mutations (W102A and D288N for TcdB; and W101A, D287N, and W519A for TcdA) were synthesized and engineered to replace corresponding GT domain genes in each wild type toxin gene. TxB-Ar was created by replacing RBD of TcdB with that of TcdA. cTxAB was generated by replacing the GT domain with that of aTcdB.

The full-length TcdA and TcdB genes were cloned into a shuttle vector pHis 1522 (pHis-TcdA and pHis-TcdB respectively) and expressed the recombinant holotoxins in B. megaterium (Yang, G et al. 2008 BMC Microbiol 8: 192). Point mutations were introduced into conserved amino acids that are associated with substrate uridine diphosphoglucose (UDP-Glc) binding, in to generate the GT-deficient holotoxins. To generate GT-mutant holotoxin A, a unique restriction enzyme (BamHI) site was installed between sequences encoding GT and CPD domains using overlapping PCR. The primer sets used were:

| (SEQ ID NO: 1) | |
|---|---|
| pHis-F, | 5′- TTTGTTTATCCACCGAACTAAG -3′, |
| (SEQ ID NO: 2) | |
| Bam-R, | 5′- TCTTCAGAAAGGGATCCACCAG-3′, |
| (SEQ ID NO: 3) | |
| Bam-F, | 5′- TGGTGGATCCCTTTCTGAAGAC -3′, |
| and | |
| (SEQ ID NO: 4) | |
| Bpu-R, | 5′- ACTGCTCCAGTTTCCCAC -3′. |

The final PCR product was digested with BsrGI and Bpu10I, and was used to replace the corresponding sequence in pHis-TcdA. The resulting plasmid was designated pH-TxA-b. Sequences encoding triple mutations (W101A, D287N, and W519A) in the GT were synthesized by Geneart (Germany) and cloned into pH-TxA-b through BsrGI/BamHI digestion. To generate the mutant holotoxin B construct, the sequence between BsrGI and NheI containing two point mutations (W102A and D288N) was synthesized and inserted into pHis-TcdB at the same restriction enzyme sites, resulting in a plasmid pH-aTcdB.

To generate the chimeric TxB-Ar, a unique RE Age I site was created at a position between TMD and RBD without change sequence of amino acids in pHis-TcdB. Then the gene encoding RBD of TcdA was amplified using primers:

TxA-Ar-F:
(SEQ ID NO: 5)
5′-AATTACCGGT TTTAACTTAGTAACTGGATGGC-3′
and
TxA-Ar-R:
(SEQ ID NO: 6)
5′-AATTGCATGCTGGTACCC TCCATATATCCCAGGGGCTTTTACT
CC-3′

and the RBD sequence of TcdB was replaced with that of TcdA through AgeI/KpnI digestion, generating a plasmid (pH-TxB-Ar). To generate the chimeric cTxAB, the XhoI/Bpu10I fragment in pH-TxB-Ar was replaced by the fragment carrying W102A and D288N mutations from pH-aTcdB.

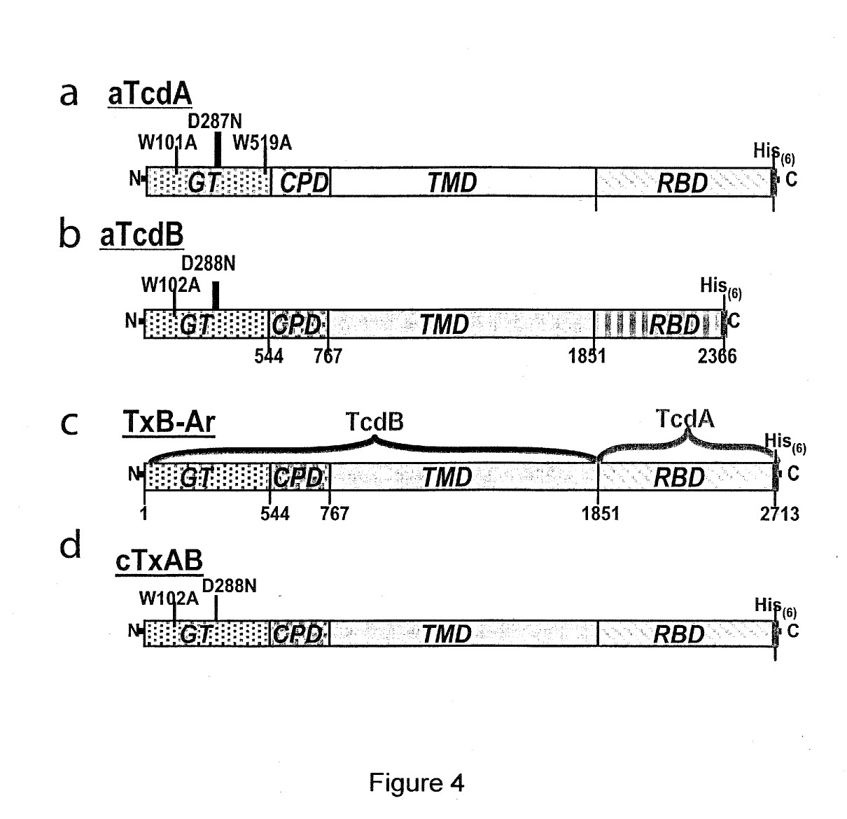

The resultant constructs carrying full-length mutant toxin and chimeric genes were used to transform B. megaterium, and mutant holotoxins (FIG. 4) were expressed and purified using the same methods described previously (Yang, G et al. 2008 BMC Microbiol 8: 192, incorporated by reference herein).

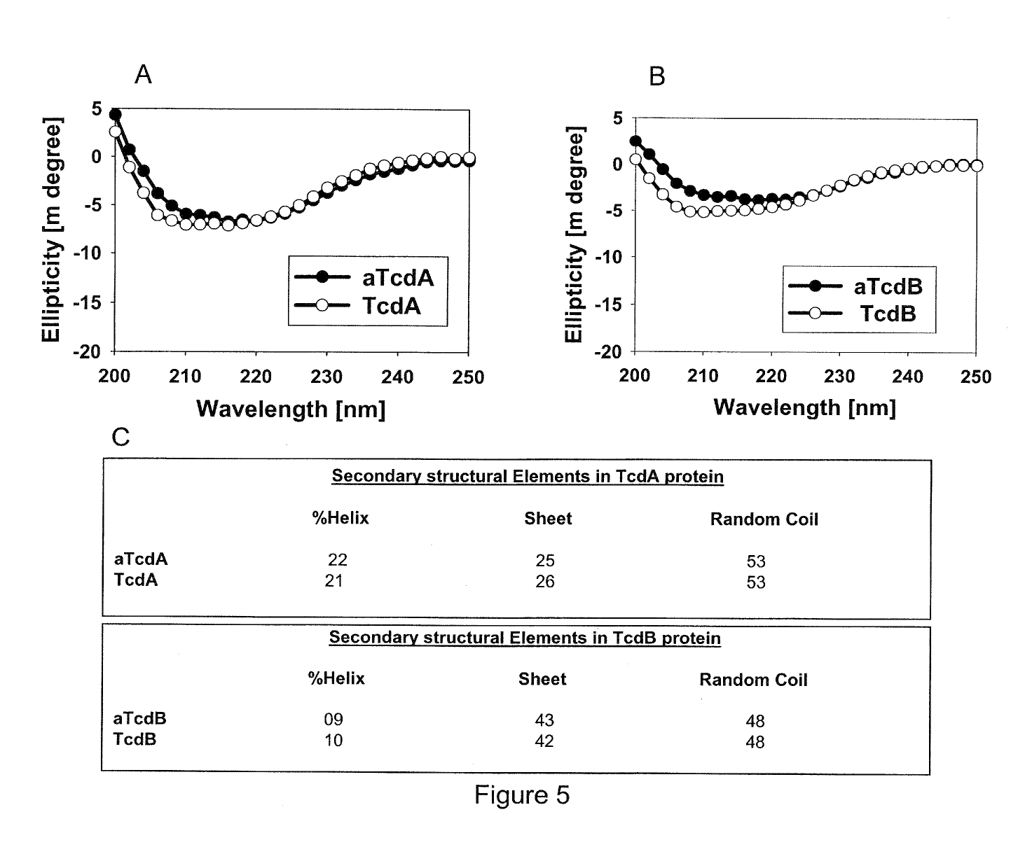

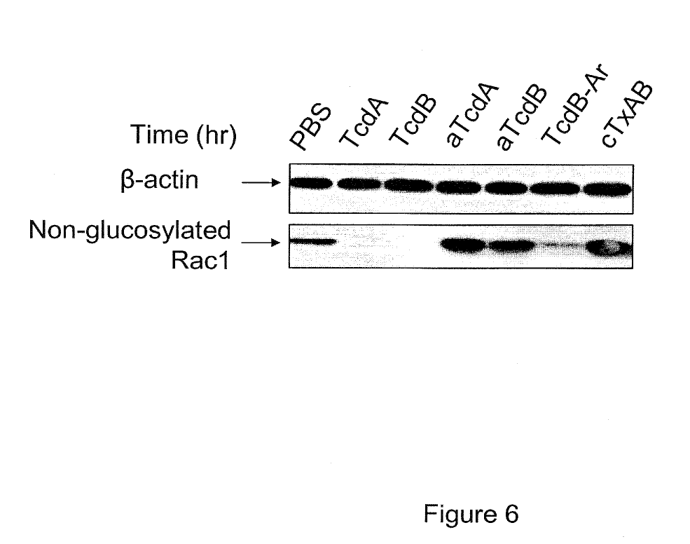

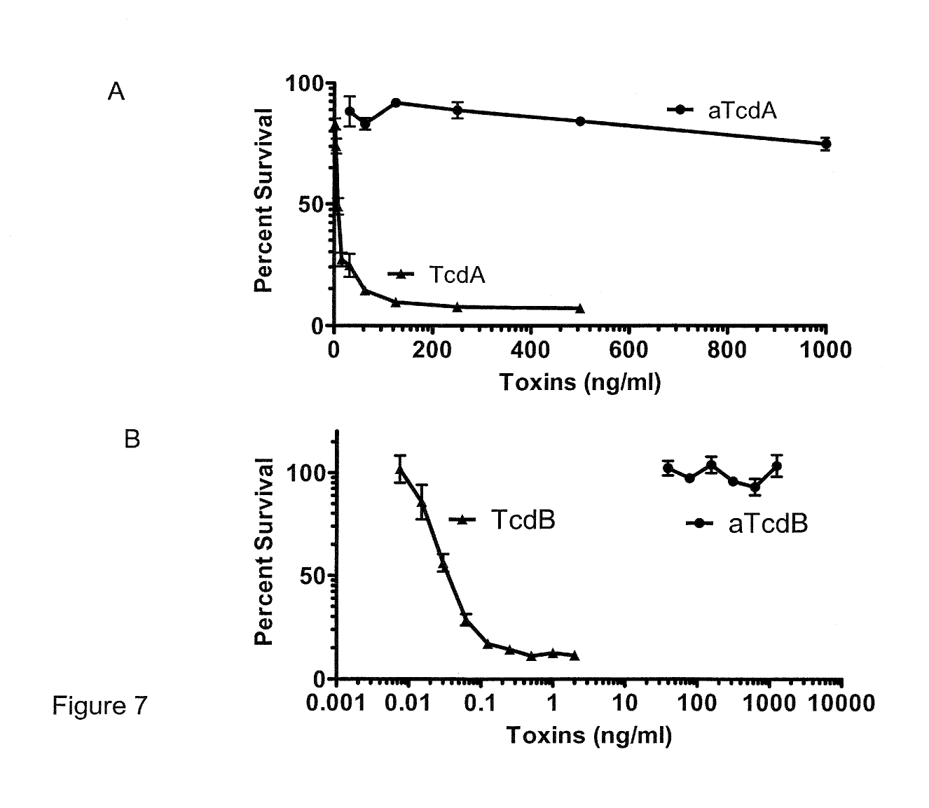

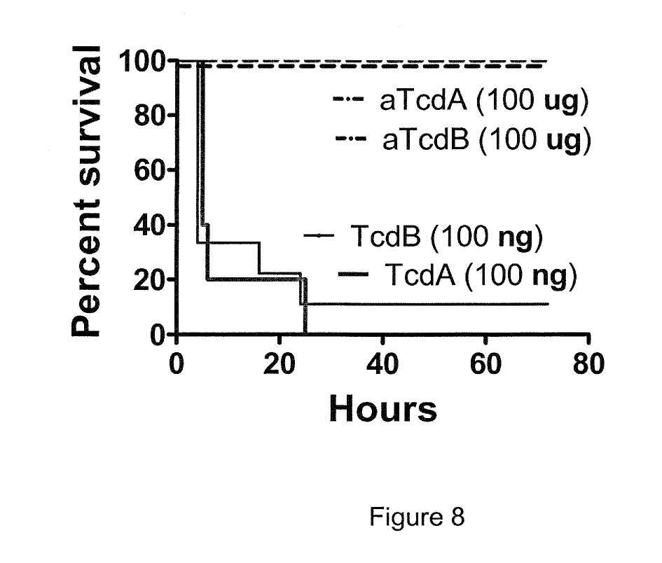

These mutant proteins were designated as aTcdA, and aTcdB respectively (FIG. 4 panels A and B). TcdA and aTcdB were observed to maintain their native structures (FIG. 5 panels A, B and C), and both mutant proteins were found to have lost virtually all glucosyltransferase activity (FIG. 6), cytotoxicity (FIG. 7 panels A and B) and in vivo toxicity (FIG. 8). Thus, the GT-deficient holotoxins herein were considered to be essentially atoxic.

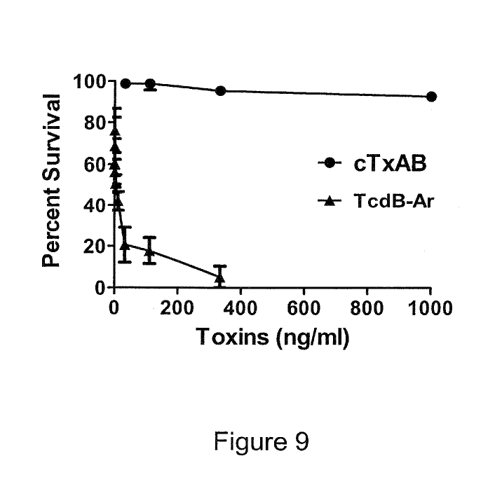

Since antitoxins against both TcdA and TcdB are necessary for full protection against CDI, a single antigen was created that is able to induce potent neutralizing antibodies against both toxins. The receptor binding domain (RBD) of TcdA is the immunodominant domain of the toxin and processes a potent adjuvant activity due to its lectin-like structure (Castagliuolo, I et al. 2004 Infect Immun 72: 2827). Therefore, the RBD of TcdB was replaced with that of TcdA, resulting in a chimeric toxin designated as TxB-Ar (FIG. 4 panel C). Surprisingly, TxB-Ar retained glucosylating activity (FIG. 6), potent cytotoxicity (FIG. 9) and exhibited strong proinflammatory activity, thus retaining characteristics of a clostridial glucosylating toxin. To engineer a GT-mutant, two point mutations (W101A and D288N) were introduced and the resultant chimeric protein was designated as cTxAB (FIG. 4 panel D). It is unlikely that the two mutations would change the overall conformation of this chimeric protein since aTcdB is structurally similar to its wild type (FIG. 5 panels B and C), thus cTxAB possesses a toxin-like conformation but remains non-toxic (FIG. 9).

### Example 5

### Safety of Immunogens

Wild type TcdB are generally much more cytotoxic than TcdA. Two point mutation in GT domain reduced the glucosyltransferease activity by up to 5 logs and the resultant aTcdB was non-toxic to mice. Mice that were challenged with 1000 times LD₁₀₀ dose of aTcdB (approximately 100 times the dose used for immunization) displayed no disease symptoms and none of them died (FIG. 3 panel C). An additional point mutation was introduced in aTcdA to ensure a total loss of toxicity. cTxAB has a deletion of D97 domain and is thus nontoxic. Therefore, both aTxAB and cTxAB are extremely safe immunogens. Because safety is important concern when designing and evaluating candidate vaccines, the safety of both aTxAB and cTxAB was further assessed by administrating mice with high doses of these proteins intraperitoneally, which allows rapid absorption of these recombinant proteins. Mice were injected with at least 100 times the established optimal immunization doses of aTxAB or cTxAB, and each immunogen was confirmed in groups of 10 mice. Mice were monitored for any abnormalities as compared with control animals. This includes loss of appetite, lethargy, loss of body weight, etc. A safety margin was established that is at least 10 times of the optimal immunization doses of aTxAB or cTxAB.

### Example 6

### Purified Recombinant Toxins from B. megaterium Devoid of Detectable Endotoxins and TLR Ligands

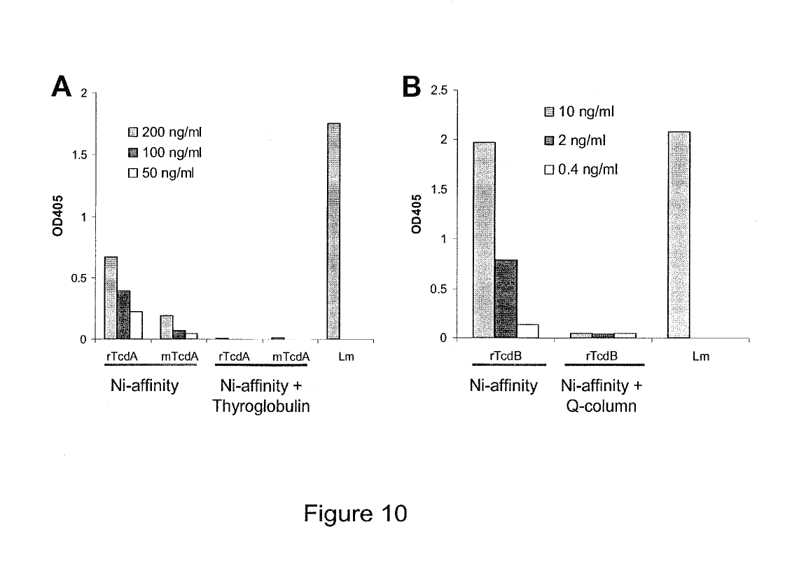

To use recombinant proteins as vaccines, it is important to obtain pure proteins free of endotoxins and other contaminants from bacteria. One of the advantages of using B. megaterium expression system is that it is endotoxin (LPS)-free. However, Gram positive bacterium is rich of TLR2 ligands which may contaminate recombinant protein preparations. Therefore, a purification scheme was here developed and highly pure recombinant toxin proteins that lacked any visible contaminant protein bands on a silver-stained SDS-PAGE was obtained (Yang, G. et al. 2008 BMC Microbiol 8:192). TLR ligand contaminations which are not visible on SDS-PAGE were assessed by highly sensitive bioassays. Engineered monoclonal hT2Y cells express human TLR2 and a secretory alkaline phosphatase (SEAP) under NF-κB promoter. Upon activation of TLR2, the cells express SEAP which can be easily measured using a phosphatase substrate. One step of His-tag affinity purification failed to eliminate TLR2 ligand contaminants (FIG. 10 panels A and B). Additional purifications (thyroglobulin-affinity and ion-exchange chromatography for TcdA and TcdB respectively) resulted in highly purified toxins that did not stimulate SEAP production. The positive control L. monocytogenes clearly induced the production of SEAP (FIG. 10 panels A and B).

B. megaterium culture occasionally is contaminated with other bacteria, such as E. coli, which are rich sources of endotoxin LPS. Therefore TLR4 contamination was examined also using the similar bioassay. Results showed that there was no detectable TLR4 ligand in recombinant toxin preparations.

Thus, the highly purified recombinant toxins contained no detectable ligands for either TLR2 or TLR4. Absence of TLR ligands, such as LPS, which cause septic shock in human indicates that toxin proteins used herein were pure and devoid of contamination.

### Example 7

### Glucosyltransferase Activity of the Toxins

The GT-activity deficiency of the mutant toxins was assessed by loss of their ability to glucosylate Rho GTPase Rac 1 in a cell-free assay. Vero cell (green monkey kidney epithelial cells) pellets were resuspended in glucosylation buffer (50 mM HEPES, pH 7.5, 100 mM KCl, 1 mM MnCl₂ and 2 mM MgCl₂) and lysed with a syringe (25G, 40 passes through the needle). After centrifugation (167,000 g, three minutes), the supernatant postnuclear cell lysate was obtained. To perform the glucosylation assay, the cell lysates were incubated with each of TcdA, TcdB, or their mutant proteins (final concentration of the toxins was 1 μg/ml) at 37° C. for the indicated time. The reaction was terminated by heating at 100° C. for five minutes in SDS-sample buffer. To determine extent of Rac1 glucosylation, lysates were separated on a 12% SDS-PAGE gel and transferred onto a nitrocellulose membrane. An antibody that specifically recognizes the non-glucosylated form of Rac1 (clone 102, BD Bioscience), or control anti-β-actin (clone AC-40, Sigma), and an HRP-conjugated anti-mouse-IgG (Amersham Biosciences) were used as the primary and secondary antibodies, respectively.

### Example 8

### Circular Dichroism (CD) Spectroscopy of Wild Type and Mutant Toxin

CD spectra were recorded on an Aviv 62 spectropolarimeter in the wavelength range of 190-260 nm, with a bandwidth of 1.0 nm and scan step of 0.5 nm using a 0.1-cm path length in a 1-cm quartz cell at 22° C. The protein concentration was in the range of 50-200 μg/ml. In each case at least five spectra was accumulated, smoothed, averaged, and corrected for the contribution of solutes.

### Example 9

### Cytopathic and Cytotoxicity Assay

Cytopathic and cytotoxic activities of the toxins were assayed as described previously (He, X et al. 2009a Infect Immun 77:294). CT-26 cells (10³/well) seeded in 96-well plates were treated with wild type or mutant toxins. To evaluate cytopathic effects of the toxins on cells, the morphological changes of cells were observed using a phase-contrast microscope. MTT assays were performed to measure the cytotoxic activities of the toxins. After 72 hours of incubation, 10 μl of MTT (5 mg/ml) were added to each well and the plates were incubated at 37° C. for another two hours. The formazan was solublized with acidic isopropanol (0.4 N HCl in absolute isopropanol), and absorbance at 570 nm was measured using a 96-well ELISA plate reader. Cell viability was expressed as the percentage of survival compared to untreated control wells. The treatments were repeated three times, and triplicate wells were assessed for cytopathic changes and cytotoxicity in each treatment.

### Example 10

### Generation of Antibodies Against TcdA and TcdB

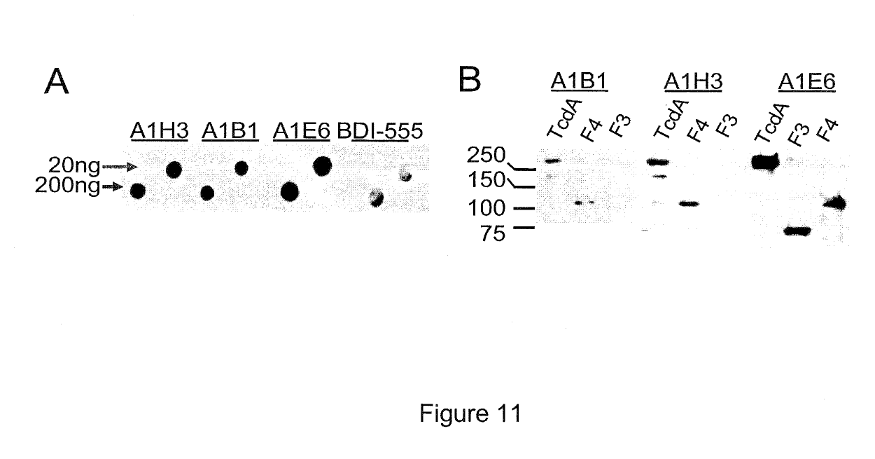

Monoclonal antibodies (mAbs) specific to TcdA were generated, including A1H3, an IgG2a isotype, and A1B1 and A1 E6, IgG1 isotypes. These antibodies recognize native TcdA (FIG. 11 panel A) and did not cross-react with TcdB.

To map the binding epitopes of these antibodies, non-overlapping gene fragments covering the full length of both lcdA and tcdB were cloned into pET32a vector. ELISA and western blotting showed that A1B1 and A1H3 recognized the TcdA C-terminal fragment F4 (from amino acid 1839 to the end), and A1 E6 recognized fragment F3 (from amino acid 1185 to 1838) and F4 (FIG. 11 panel B). A1H3 can substantially enhance TcdA cytotoxicity on FcγRI-expressing cells, where as A1 E6 and A1B1 have no such activities (He, X. et al. 2009a Infect Immun 77:2294-2303, incorporated by reference herein in its entirety).

Several IgG and IgM mAbs against TcdB were generated and rabbit anti-toxin polyclonal antibodies were generated against either TcdA or TcdB. The antigens used to generate these antibodies were highly purified rTcdA or rTcdB. The polyclonal antibodies bind specifically to native toxins and neutralize their cytotoxicity activities (He, X. et al. 2009a Infect Immun 77:2294-2303, incorporated by reference herein in its entirety). These monoclonal and polyclonal antibodies were used for various assays shown in Examples herein.

### Example 11

### Mouse Ileal Loop Model to Analyze TcdA-Induced Enterocolitis

TcdA administrated intragastrically induces severe enterocolitis in animals, and TcdB has no enterotoxicity in animals (Lyerly, D. M. et al. 1985 Infect Immun 47:349-352). To analyze TcdA-induced enterocolitis, a mouse ileal loop model was established ollowing previously reported methods (Cavalcante, I. C. et al. 2006 Infect Immun 74:2606-2612).

TcdA induced fluid accumulation and histological alterations in a ligated mouse ileal loop in CD1, 129SV, NIH Swiss, Balb/c, and C57BL/c mice with variable sensitivities among the different mouse strains. TcdB has no enterotoxicity in mice and induces no inflammation or tissue damage in ligated loops.

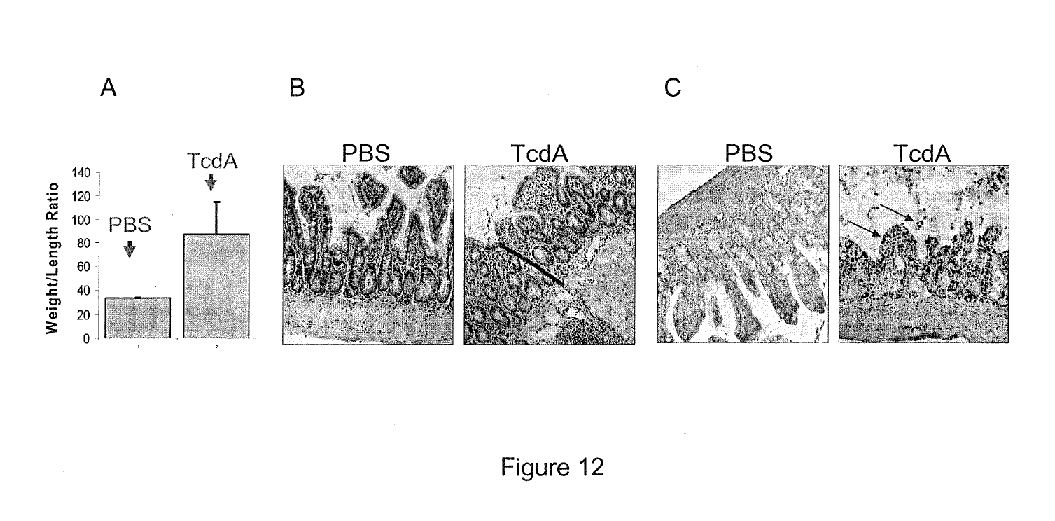

To determine whether intestinal TLR ligands contribute to the inflammatory response, Myeloid differentiation factor 88 (MyD88) knockout mice were utilized which do not respond to the most TLR signaling (Dunne, A. et al. 2003 Sci STKE 2003:re3). MyD88 knockout mice here were found to be as sensitive as wild type (C57BL/6) to TcdA-induced enteritis. Injection of 50 μg of recombinant TcdA into a ligated ileal loop of MyD88 knockout mice induced inflammatory response in the intestine (FIG. 12). Significant fluid accumulation was seen in ileal loops injected with TcdA, but not PBS controls (FIG. 12 panel A). Histopathological examination showed damaged villi and influx of immune cells (FIG. 12 panel B) and neutrophils with some migrating into the intestinal lumen (indicated by the arrows in FIG. 12 panel C) after TcdA, but not PBS injection. The intestinal fluid accumulation, inflammation, and mucosal damage were completely blocked by rabbit antisera against TcdA. These data shows that MyD88 adaptor protein plays no role in TcdA-mediated enteritis in mice.

### Example 12

### Disease Manifestation in GB Piglet Model Resembled Symptoms of CDI in Humans

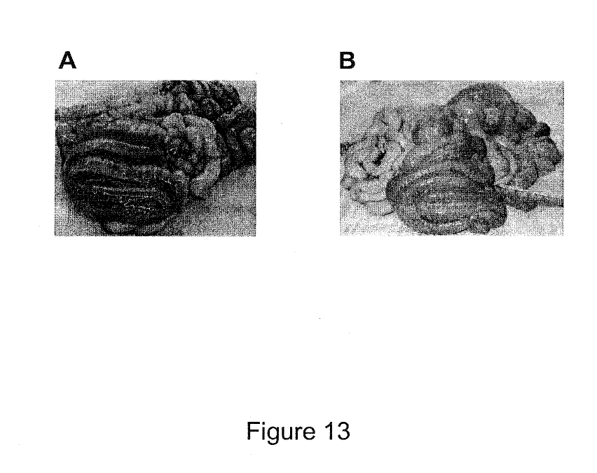

A gnotobiotic piglet model was established that closely reflects the mucosal abnormalities of C. difficile associated colitis (Steele, J. et al. 2010 J Infect Diseases 201:428). Piglets challenged at two days of age with 10⁶ spores or 10⁸ vegetative cells developed acute diarrhea within two to three days, with dramatic lesions of mesocolonic edema extending from the ileocolic junction to the rectum, and the spiral colon was often distended and hemorrhagic with focal necrosis of the mucosa. Animals challenged at five to seven days of age with a lower dose developed a more chronic disease with typical pseudomembranous colitis, inflammation and profound mucosal damage, manifested with prolong inteimittent diarrhea and weight loss. These disease manifestation characteristics resembled symptoms of human CDI. Thus, the piglet presents a useful model to assess mucosal protection of candidate vaccines against chronic diseases (FIG. 13 panels A and B).

### Example 13

### Protection Against Systemic Toxin Challenge

Protection against systemic toxin challenge was performed. LD₅₀ᵢ was used as the standard challenge dose established in Examples herein to assess the levels of the protection against systemic toxin challenge induced by the mucosal immunization for each immunogen. The mucosal immunization was observed to induce a similar level of protection as do parenteral immunization, in which 50% of mice survived from challenge with LD₅₀ᵢ dose of each wild type toxin, or two toxins given together. A dose optimization was performed if greater than 50% of mice were observed to die.

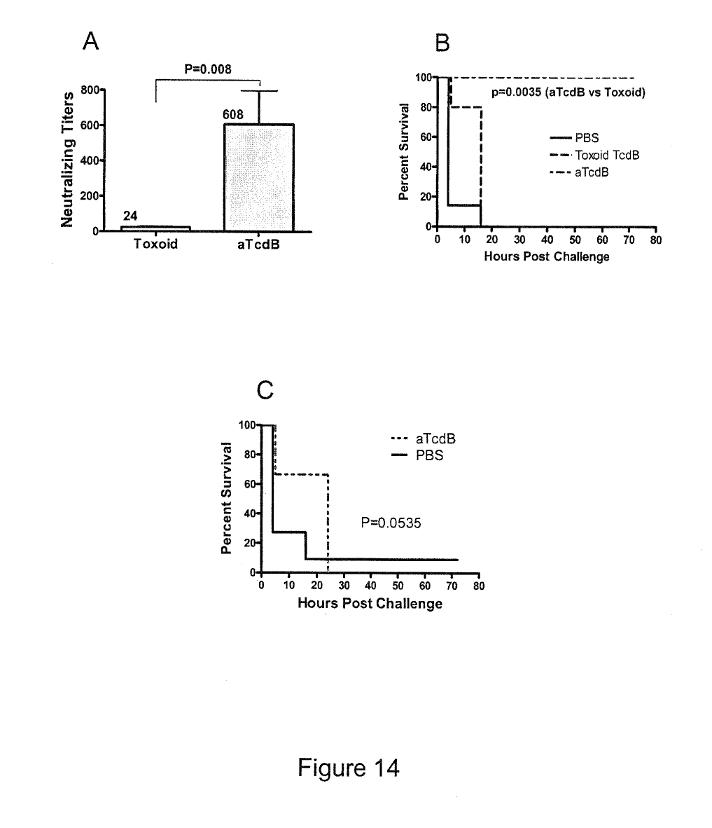

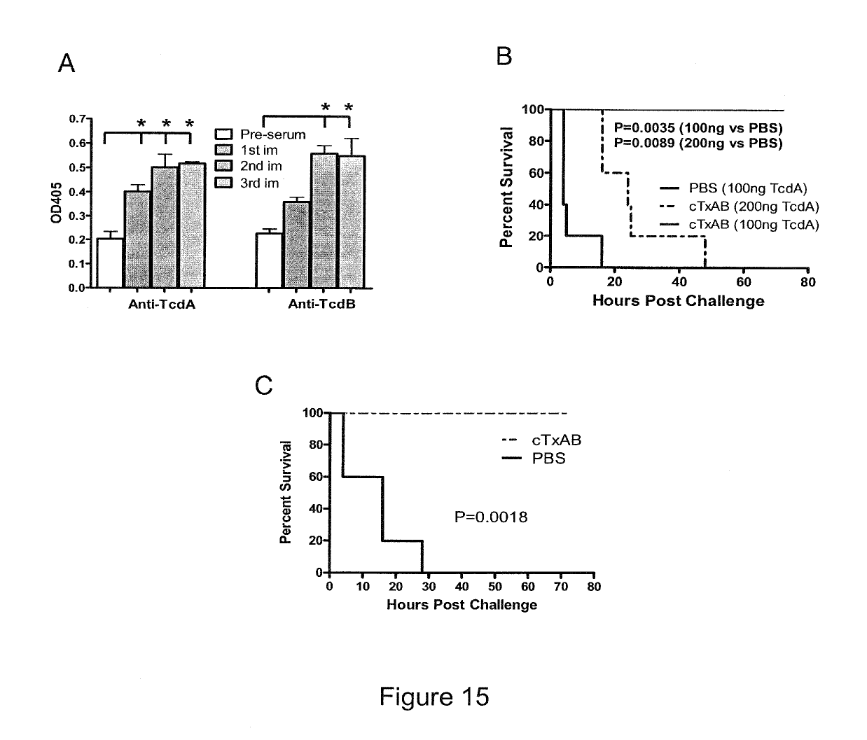

A potent antibody response was generated that protects mice against challenge with a lethal dose of wild type toxin after the aTxAB- or cTxAB-immunized mice (body weight around 20 g) are challenged IP with a lethal dose of either wild type of TcdA, TcdB, or a mixture of TcdA and TcdB (100 ng for each toxin), one week after the last immunization. Mice immunized with aTcdB were observed to be fully protected against challenge of a lethal dose of wild type TcdB, and not TcdA (FIG. 14 panels B and C). Further, vaccination with cTxAB was observed to protect mice from lethal challenge by either toxin (FIG. 15 panels B and C). Mice immunized with a mixture of aTcdA and aTcdB were, based on data herein, expected to be fully protected from the activity of both toxins, and this protection from challenge of each of the toxins was observed in vivo.

### Example 14

### Serum Neutralizing Titers and In Vivo Protection Resulting from aTcdB Immunization

To assess whether sera from aTcdB immunized mice was capable of neutralizing the cytotoxicity of TcdB in cultured cells, mouse intestinal epithelial cell line CT26 which is highly sensitive to TcdB was used. The neutralizing titer is defined as the reciprocal of the maximum dilution of serum that fails to block cell rounding induced by toxin at a given concentration. This concentration is four times the minimum dose of the toxin that causes all CT26 cells to round after a 24-hour TcdB treatment. This minimum dose of TcdB causing 100% of CT26 cells rounding after 24 hours of toxin treatment (0.0625 ng/ml) was identified. Therefore, an amount that is four times the minimum dose (0.25 ng/ml) of TcdB was mixed with two-fold diluted serum samples which were then applied to CT26 cells.

Data obtained after 24 hours of incubation showed that sera from aTcdB immunized mice lost blocking activity as diluted in 1 to 608 in average (n=5). Therefore, the calculated neutralizing titer of sera from aTcdB-immunized mice was 608. In contrast, that of sera from toxoid-immunized mice was 24 (FIG. 14 panel A). Thus, the neutralizing activity of antibodies induced by aTcdB immunization was significantly higher than that induced by toxoid.

Further, mice immunized with aTcdB were completely protected from lethal IP challenge with wild type TcdB (FIG. 14 panel B). This anti-TcdB immunity was observed to be long lasting since mice survived a rechallenge with TcdB even two months after the last immunization. This protective immunity was observed to be specific since the immuned mice did not survive a challenge with a lethal dose of TcdA (FIG. 14 panel C). Toxoid immunization provided significantly lower protection than aTcdB immunization. Although toxoid-immunized mice survived longer than control PBS treated non-immunized mice, all succumbed within 24 hours (FIG. 14 panel B). These data also show that although TcdA and TcdB are highly similar in both amino acid sequence and domain structure, they are antigenically distinct with little cross reactivity between them. Therefore, a vaccine that induces antibodies against both toxins is necessary to provide full protection.

### Example 15

### CTxAB Immunization Induced Antibody and Protective Responses Specific for Both TcdA and TcdB

The receptor binding domain (RBD) of TcdA contains multiple lectin-binding repeats and has potent immunostimulatory and adjuvant activities (Castagliuolo, I. et al. 2004 Infect Immun 72:2827-2836; Yeh, C. Y. et al. 2008 Infect Immun 76:1170-1178). TcdB is a virulent factor for C. difficile. For these reasons, a chimeric toxin protein was designed by replacing RBD of TcdB with RBD of TcdA (FIG. 2 panel B). In addition, a small deletion (97 amino acids which is essential for the toxicity of TcdB) in the C-terminus of TMD was created therefore the resultant cTxAB is non-toxic (FIG. 2 panel B).

Immunization of mice with cTxAB was observed to induce IgG antibodies responses against both TcdA and TcdB (FIG. 15). Significant amount of anti-TcdA and anti-TcdB were induced after a first booster, and a second booster did not increase the magnitude of the antibody responses against either TcdA or TcdB (FIG. 15 panel A). Surprisingly, immunization of mice with cTxAB provided full protection against lethal systemic challenge with either TcdA or TcdB. All cTxAB-immunized mice survived the IP challenge of 100 ng of either TcdA or TcdB, and all the placebo-immunized mice died (FIG. 15 panels B and C). Increasing the challenge dose of TcdA to 200 ng led to mortality of the cTxAB-immunized mice, but these mice survived significantly longer when compared to control mice challenged with 100 ng of TcdA (p=0.0089) (FIG. 15 panel B).

### Example 16

### The GT-Deficient Holotoxins (aTcdA and aTcdB) Bind and Enter into Cells Equally Well to Wild Type Toxins

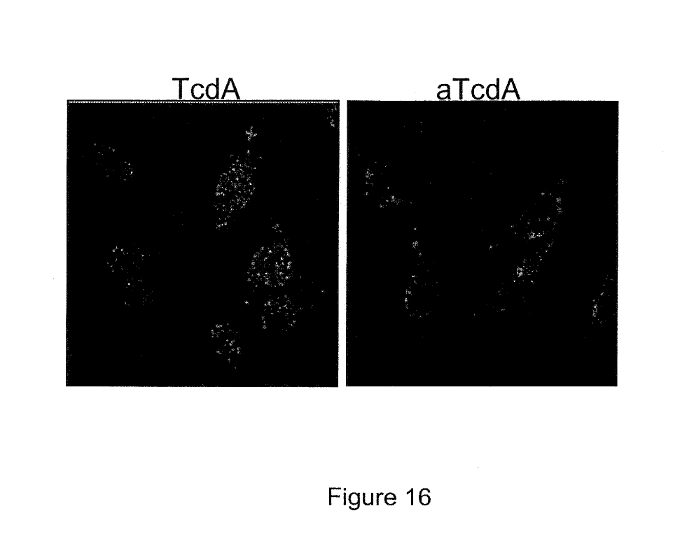

Because aTcdA and aTcdB have only two or three point amino acid changes which are located in GT domain and the receptor binding domains are intact and unaltered, these proteins have affinities to cell surface that are similar to wild type proteins. At 4° C., aTcdA was observed to bind to RAW264.7 cells equally well as wild type TcdA, as determined by immunofluorescence staining. Exposing the toxins to RAW264.7 cells for 30 minutes at 37° C., temperature permitting endocytosis, resulting in each of aTcdA and TcdA becoming internalized comparably into the RAW264.7 cells, as determined by specific monoclonal antibody (A1H3) staining following a confocal microscopy analysis (FIG. 16). These data show that the point mutations at GT domain do not affect the cellular binding and internalization of the holotoxins. The chimeric toxin cTxAB has an intact RBD of TcdA, thus it binds to culture cells similarly to wild type TcdA (FIG. 3 panel D).

### Example 17

### Parenteral Immunization and Antibody Response

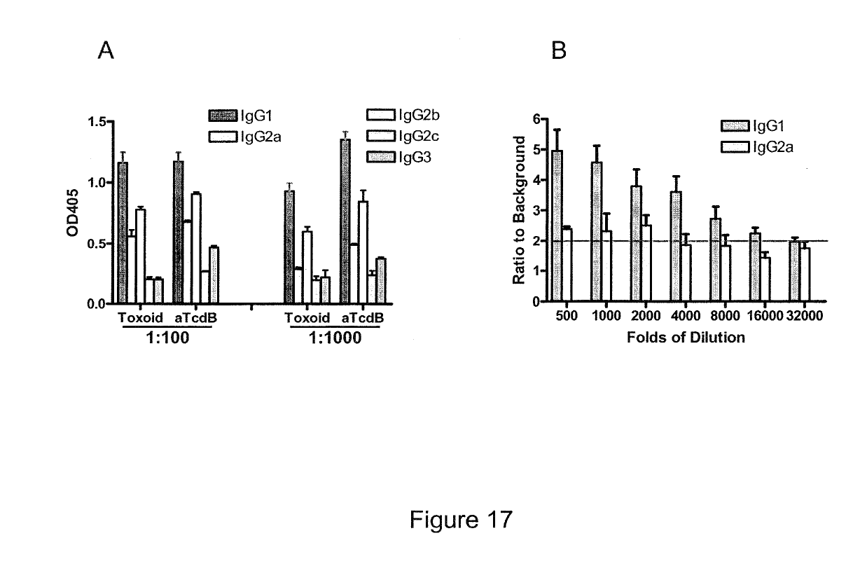

Groups of mice are immunized IP with a dose of aTxAB, cTxAB, or toxoid (a mixture of TcdA and TcdB), with alum as an adjuvant. Each of IP immunization and subcutaneous immunization is performed, and similar results are expected to be obtained from these routes. Both toxoids and aTxAB contain an equal amount of each toxin proteins, and the initial dose is 5 μg of total protein(s) per injection, which follows the dose used in the prior immunization. The serum samples are collected and the anti-toxins (both TcdA and TcdB) responses are measured using standard ELISA as described in Examples herein. Several parameters are evaluated among the groups: the speed and magnitude of the antibody response, the IgG subtypes and IgG1/IgG2a ratios. aTcdB immunization induced primarily IgG1 response (FIG. 17). Without being limited to any particular theory or mechanism of action, immunization with either aTxAB or cTxAB is expected to induce predominantly IgG1 responses. Analysis of possible antibody response to His₆ tag and StrepTag is performed. Both tags have a very small size and therefore are likely to be less immunodominant. Using purified native TcdA and TcdB, antibodies specific for the toxins are differentiated from those specific for the tags. The irrelevant recombinant antigens with the tags also helps differentiate between the two. The tags can also be removed by thrombin protease since a thrombin recognition site was installed in cTxAB (FIG. 3 panel B). The thrombin recognition sequence can be similarly installed into aTcdA and aTcdB as appropriate.

### Example 18

### IgG Subclasses Induced by aTcdB Immunization

IgG subclasses induced by aTcdB or toxoid immunization were measured. Immunization of mice with either aTcdB or toxoid induced significant anti-TcdB IgG1 and IgG2b responses. In contract, the responses of other IgG subtypes such as anti-TcdB IgG2a, IgG2c, and IgG3 were low (FIG. 17 panel A). Serial dilutions of sera from aTcdB-immunized mice resulted in loss of titer of anti-TcdB IgG1 at 1:32,000 dilution, and anti-TcdB IgG2a was only down to 1:2000. These data show that the immunogen aTcdB, given IP, induced mainly TH-2 type response.

### Example 19

### Immunization of Mice with aTcdB Induced Rapid IgG Response

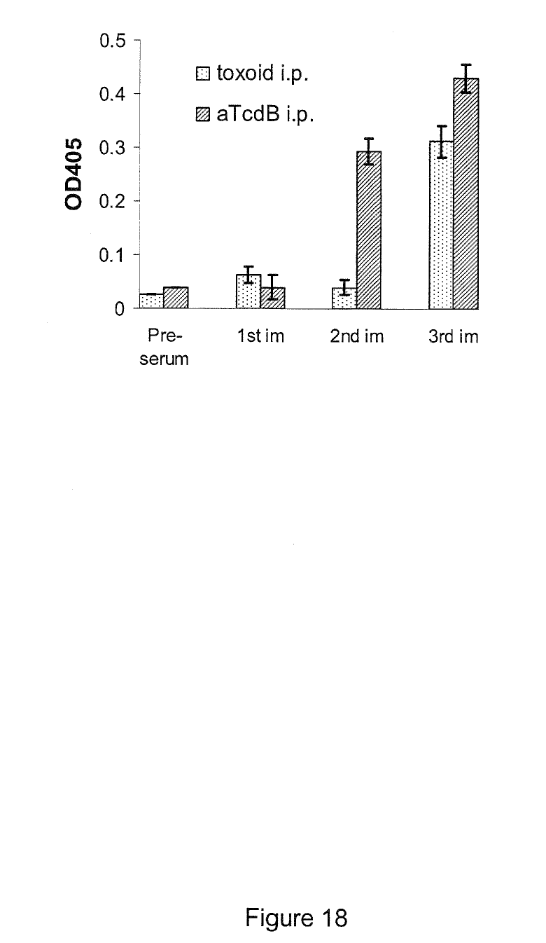

People at high risk of C. difficile infection, such as under antibiotic treatment and/or hospitalization, are subjects for prophylactic vaccination. A vaccine capable of inducing rapid protective immunity is desirable especially in hospitalized patients. Vaccination with aTcdB was examined for capability to induce rapid antibody response. FIG. 18 shows that immunization of mice with aTcdB generated a potent IgG response after the second immunization. In contrast, toxoid immunization generated a detectable IgG response only after the third immunization (on day 28 after priming).

### Example 20

### Antibodies Generated by aTcdB Immunization React to Both N- and C-Termini of the Holotoxin

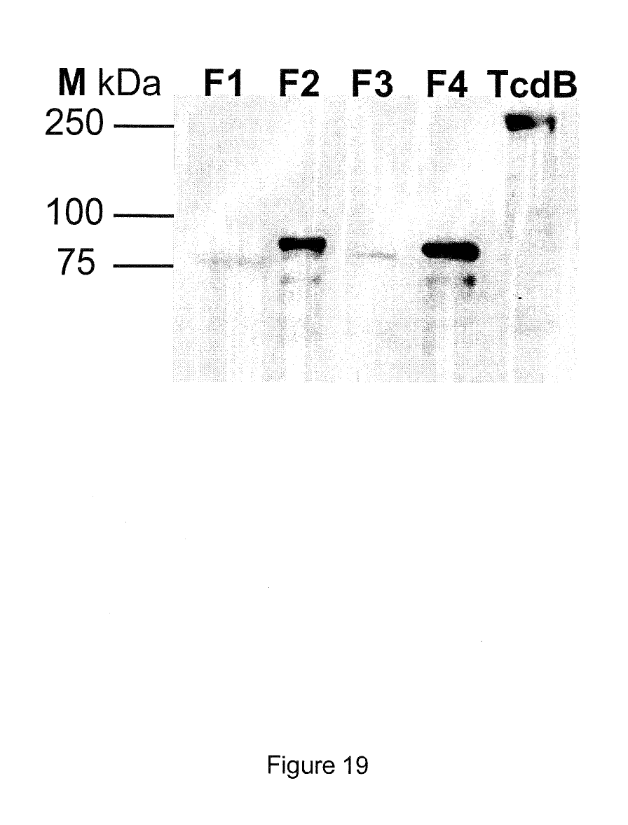

Because of the large size and toxicity of C. difficile toxins, recombinant toxin fragments that lack the GT domain and that consequently are non-toxic are likely to be potential vaccine candidates. The C-terminal fragment containing full or partial receptor binding domain of TcdA has been reported to be immunodominant and therefore used as immunogen for inducing anti-toxin antibody response (Kink, J. A. et al. 1998 Infect Immun 66:2018-2025; Sauerborn, M. et al. 1997 FEMS Microbiol Lett 155:45-54; Ward, S. J. et al. 1999 Infect Immun 67:5124-5132). However, the N-termini of those holotoxins with enzymatic domains also are capable of inducing neutralizing antibodies (Babcock, G. J. et al. 2006 Infect Immun 74:6339-6347). Immunization of mice with aTcdB generated antibodies with epitopes specific for F1 through F4 fragments that are found throughout holotoxin (FIG. 19). Thus, aTcdB vaccination of mice induced a wide-spectrum of antibody responses.

### Example 21

### Antibody and Protective Responses of Mice Immunized with aTxAB or cTxAB

The antibody and protective responses of mice immunized parenterally with aTxAB or cTxAB are investigated and compared with the formalin-inactivated wild type TcdA and TcdB (toxoids). Groups of mice are immunized intraperitoneally (IP) or subcutaneously (SC) three times with a given dose of aTxAB, cTxAB, or toxoids mixed with alum as the adjuvant. Serum antibody responses are measured after each immunization. One week after the last immunization, mice are challenged IP with wild type toxins and the protective responses to challenge are compared with a placebo (vehicle plus adjuvant) immunized group. Dose optimization of the immunogens is performed followed by doubling and halving the dose given to mice, and the lowest amount of antigen required to induce the highest level of serum antibody response for each immunogen is established. The safety of the two immunogens is evaluated by challenging mice with 10 to 100 fold of the optimal immunization doses established after dose optimization, monitoring for signs of toxicity and other abnormalities, including fatalities. The immunized mice are challenged with wild type toxins to establish the corresponding LD₅₀ᵢ for each toxin and for the combination of the two. Cytotoxicity assays are performed to determine the antibody neutralizing titers against each wild type toxin. These assays establish the optimal immunization dose accomplished with systemic immunization, and the calculated LD₅₀ᵢ toxin challenge dose required for this level of protection; the atoxic forms are expected to be more efficient than the toxoid.

### Example 22

### Immunization with aTcdB Induced Greater IgG Response than Toxoid

Formaldehyde-inactivated toxins (toxoid) have been used as vaccine and proved to be effective against C. difficile associated diseases in animal models (Sougioultzis, S. et al. 2005 Gastroenterology 128:764-770; Torres, J. F. et al. 1995 Infect Immun 63:4619-4627). Without being limited to any particular theory or mechanism of action, the data herein show that the toxoid forms of C. difficile toxins lack conformational antigens compared to native holotoxins, and to the atoxic holotoxins as well.

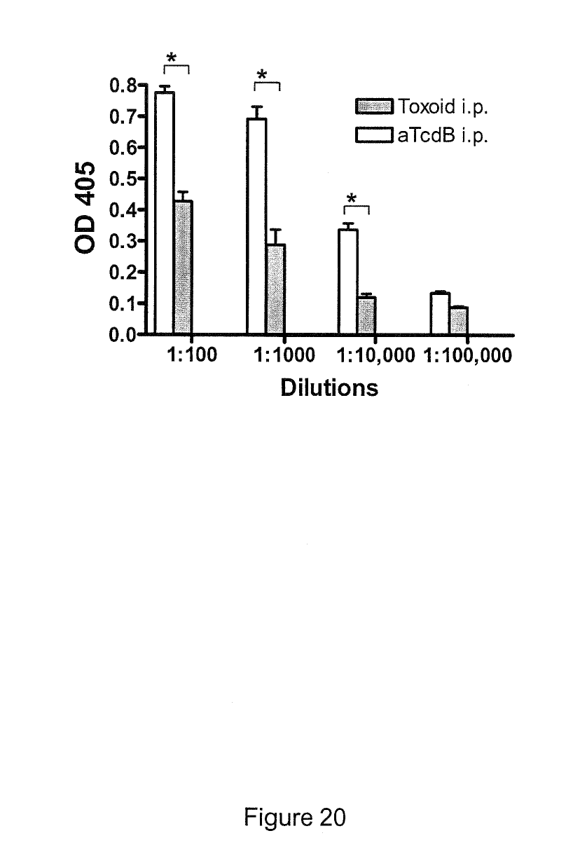

Toxoid TcdB with aTcdB were compared for their abilities to induce antibody response. Mice were immunized with equal amount (5 μg per injection) of toxoid or aTcdB with alum as adjuvant. After three immunizations, ELISA results of anti-TcdB IgG (FIG. 20) show that aTcdB induced a significantly higher amount of toxin-specific IgGs than did toxoid.

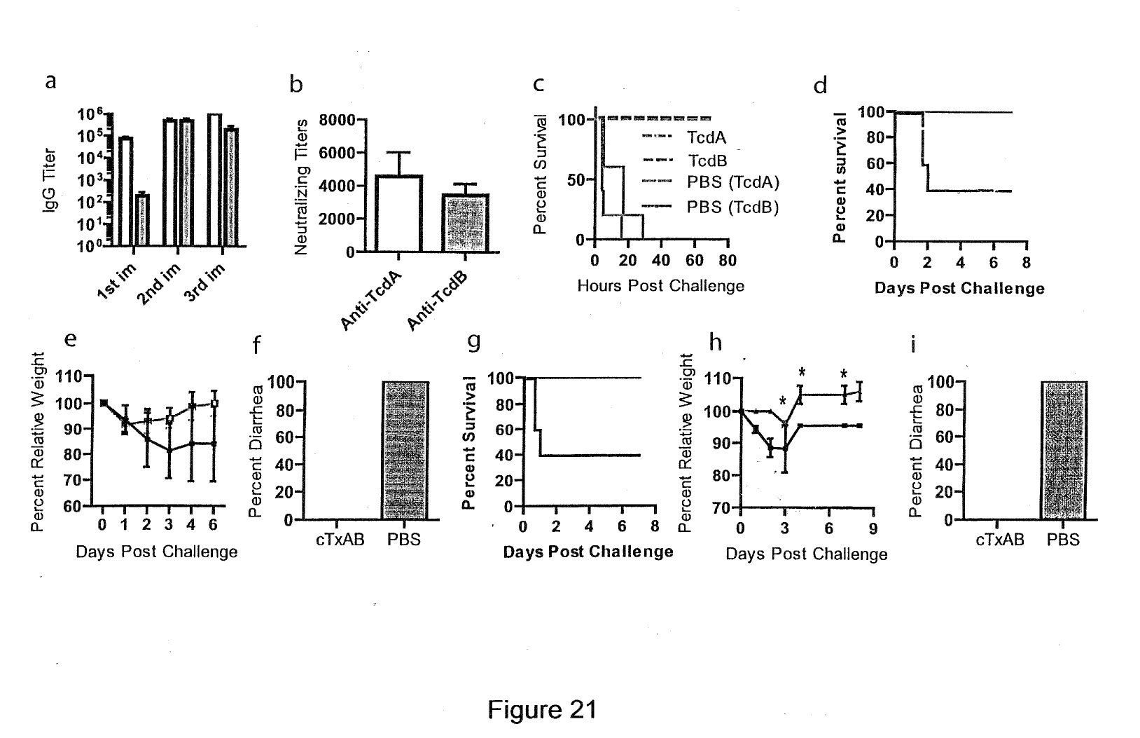

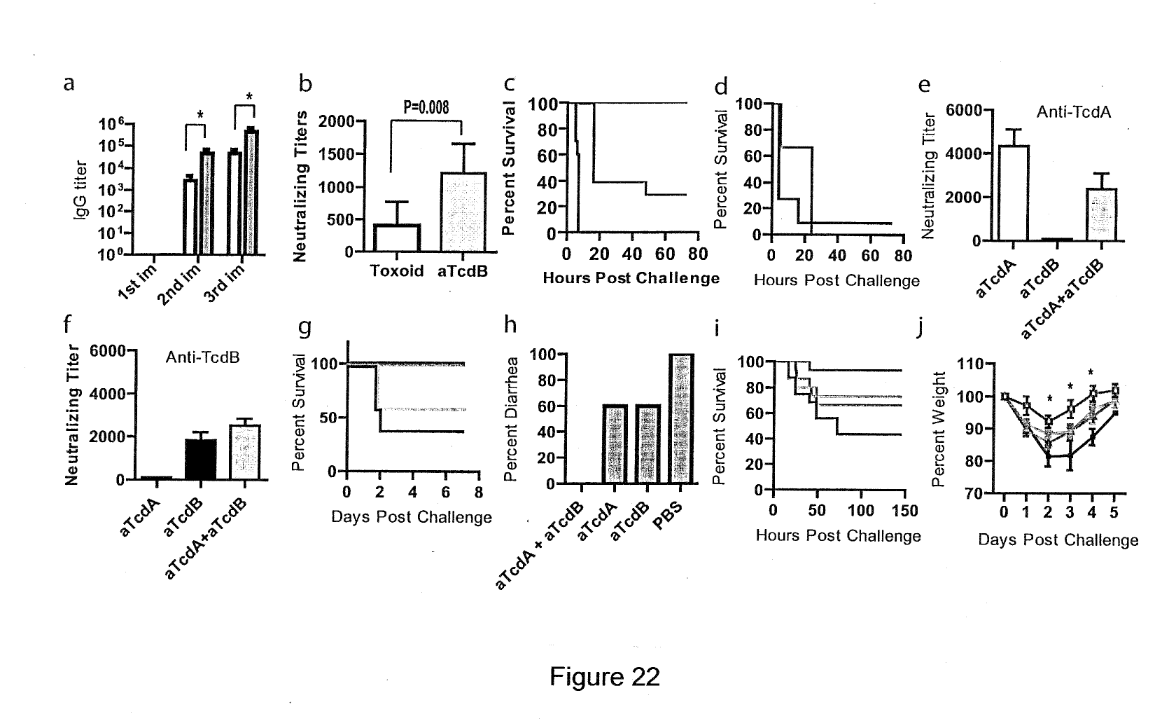

Studies highlighted the importance of TcdB as a major virulence factor of C. difficile. Immunogenicity of aTcdB was compared to that of toxoid TcdB (toxoid B). Systemic immunization with aTcdB induced a stronger IgG response and substantially higher neutralizing activity than did toxoid B (FIG. 21 panels A and B). aTcdB-immunized mice were fully protected against lethal wild type TcdB challenge whereas all toxoid B-immunized mice showed signs of systemic infection and 70% of mice succumbed within 48 hours (FIG. 22 panel C). Thus, aTcdB immunization induced significantly better protection from systemic toxin challenge than did toxoid B (p=0.0014).

Because of the high immunogenicity of aTcdB and significant homology between toxins TcdA and TcdB, antibodies generated by aTcdB immunization were examined for cross-protection against TcdA. Surprisingly, antibodies generated by aTcdB immunization had little neutralizing activity against the cytotoxicity of TcdA and failed to protect mice from lethal TcdA challenge (FIG. 22 panels D and E). Immunization with aTcdA also induced little neutralizing antibody response against TcdB (FIG. 22 panel F). However, mice immunized with a mixture of aTcdA and aTcdB, generated potent neutralizing antibodies against both toxins (FIG. 22 panels E and F). These mice were fully protected against lethal systemic challenge with either TcdA or TcdB. Furthermore, immunization of mice with either aTcdA or aTcdB alone partially protected mice against oral C. difficile bacterium-induced CDL aTcdA and aTcdB together as immunogens induced full protection against the lethality and diarrhea of CDI (FIG. 22 panels G and H). Passive immunization of mice by intraperitoneal administration of polysera antitoxins against both toxins provided significant protection/therapy against C. difficile-induced mortality and weight loss, compared yo polysera against each toxin alone which were only partially protective (FIG. 22 panels I and J).

### Example 23

### CTxAB Enteral Immunization of Mice Induced Superior Neutralizing Activities that Blocked Cytotoxicity of Both TcdA and TcdB

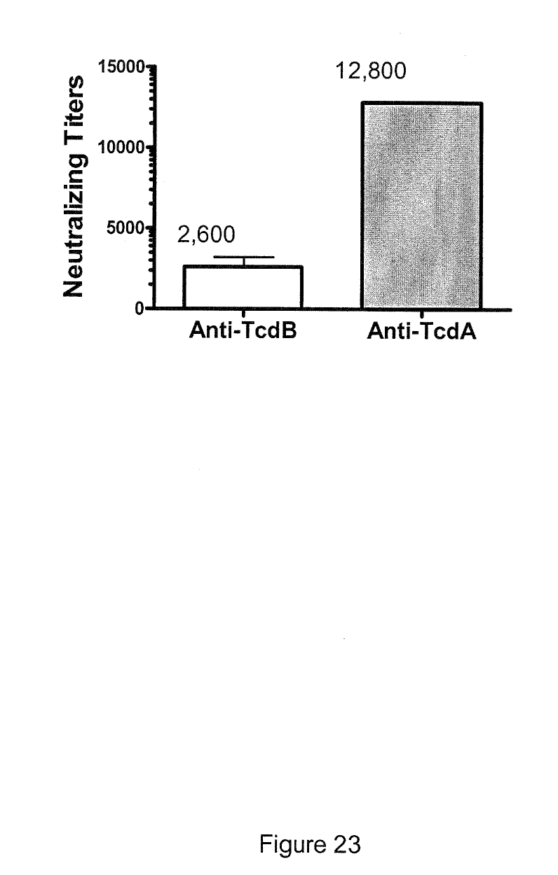

To examine whether cTxAB as vaccine induces neutralizing antibodies after parenteral immunization, mice were immunized IP four times (every ten days) with alum as adjuvant. The serum samples were collected seven days after the last immunization and neutralizing titers were measured. Sera from cTxAB-immunized mice was observed to have superior neutralizing activity against both TcdA and TcdB. Even at very high dilutions, the sera still demonstrated neutralizing activities (FIG. 23).

Neutralizing activity against TcdA in cultured cells was observed to be much greater than that against TcdB (FIG. 23). Without being limited to any particular theory or mechanism of action, this may be due to cTxAB immunization ability to induce antibodies capable of blocking the receptor binding domain (RBD) of TcdA, which is needed for the binding and subsequent toxicity the toxins to cultured cells. The data show that the antibodies induced by cTxAB had potent abilities to block toxicity of both toxins in vivo.

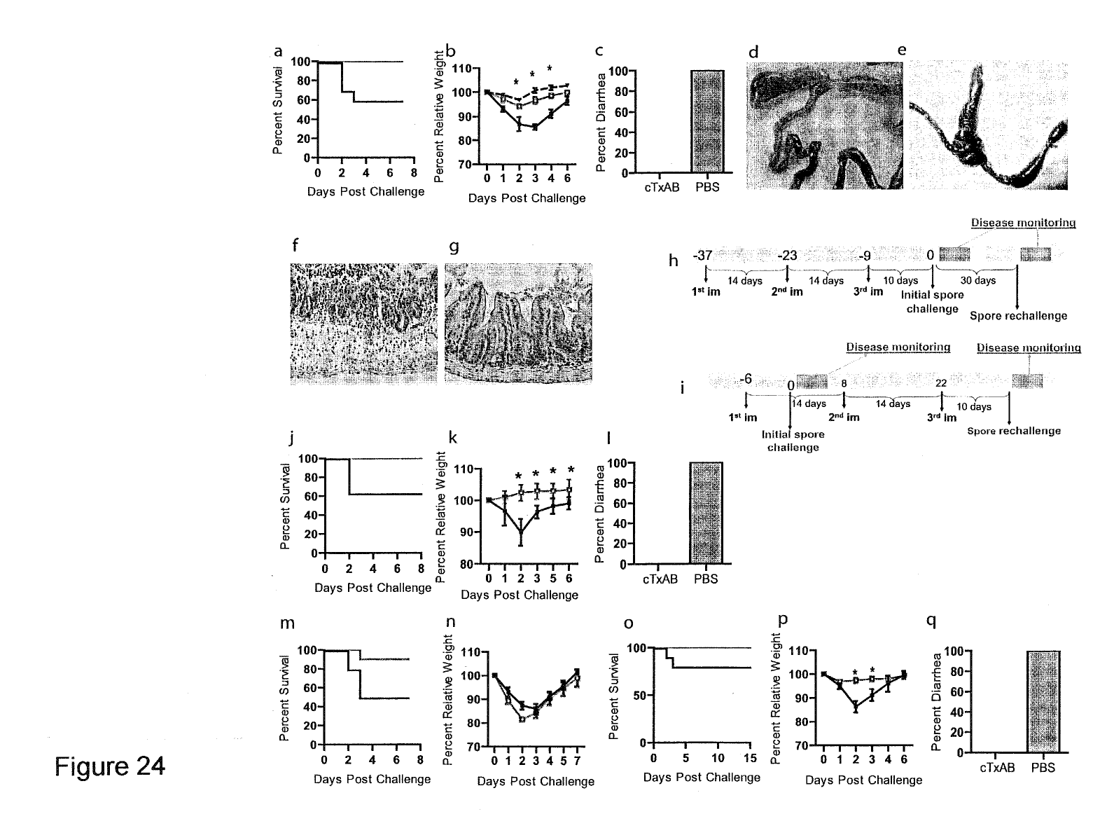

A cTxAB candidate vaccine was evaluated for capability to induce protection against a challenge with the hypervirulent strain. After three immunizations with cTxAB, mice were challenged with C. difficile spores of the UK1 strain, a 027/B1/NAP1 strain isolated from a patient (Steele, J et al. 2010 J Infect Dis 201: 428). The control PBS non-immunized mice started to exhibit signs of disease (ruffled coat, lethargy, loss of appetite, weakness, etc) on day 1, developed severe diarrhea on day 2 after infection, and approximately 40% of mice succumbed (FIG. 24 panels A, B and C). In contrast, the cTxAB-immunized mice were fully protected, and showed no sign of disease even months after infection (FIG. 24 panels A, B and C).

Immunization of mice with cTxAB was observed to induce potent systemic antibody responses against both TcdA and TcdB (FIG. 21 panel A). Significant IgG response was generated after a single immunization (FIG. 21 panel A), a result that may be due to the immunostimulatory effect of RBD of TcdA since aTcdB did not induce a measurable antibody response after a single immunization (FIG. 22 panel A). Despite the potent systemic antibody response, immunization of mice with cTxAB resulted in low intestinal antitoxin IgG and IgA titers measured in feces of the immunized mice. After three immunizations with cTxAB, the serum neutralizing titers against TcdA and TcdB were observed to be 4560 and 3440 respectively (FIG. 21 panel B). The immunized mice after these rounds were fully protected against lethal systemic challenge with either wild type TcdA or TcdB (FIG. 21 panel C).

Since the chimeric protein was found to be capable of inducing potent neutralizing antibodies against both toxins, ability of cTxAB vaccination to induce a protective response against CDI was examined herein. Mice were subjected to three rounds of immunizations and oral challenge by vegetative cells of the laboratory C. difficile strain VPI10463, vehicle PBS-immunized mice developed typical diarrhea and displayed weight loss, and approximately 60% of mice succumbed (Chen, X et al. 2008 Gastroenterology 135: 1984) (FIG. 21 panels D and E). However, none of the mice immunized with cTxAB developed symptoms of disease, although a slight weight loss was observed (FIG. 21 panels D and E). None of the cTxAB-immunized mice developed diarrhea. In contrast all mice that were administered PBS control developed severe diarrhea (FIG. 21 panel F) within 3-4 days after infection and gradually recovered within a week. The protection induced by cTxAB vaccination was long-lasting, and the immunized mice were fully protected against a C. difficile challenge with bacteria even three months from the third immunization (FIG. 21 panels G, H and I).

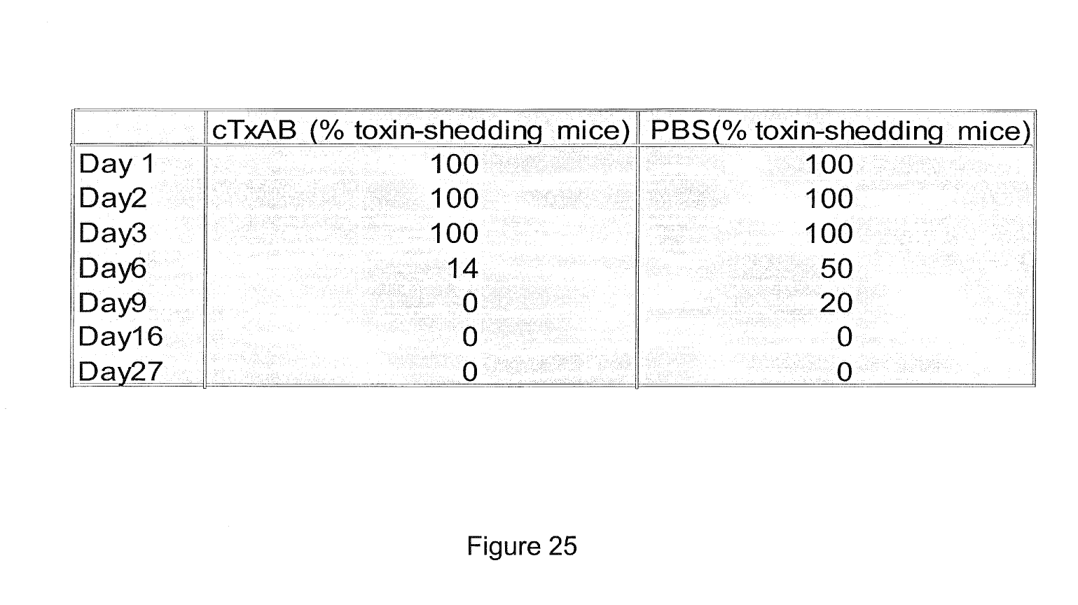

The cTxAB immunized mice exhibited no sign of CDI, and these mice shed detectable amounts of both toxins for one week, at levels similar to PBS-treated control mice (FIG. 25). The mice were further examined for any intestinal lesion or damage caused by the toxins. Necropsy data showed that the ceca and colon from PBS treated control mice were significantly enlarged and swollen (FIG. 24 panel D), and those organs from cTxAB immunized mice appeared normal (FIG. 24 panel E). Thin sections of the ceca from PBS control mice displayed significant epithelial damage, edema, and infiltration of immune cells (FIG. 24 panel F) while cTxAB immunized mice showed no evidence of mucosal damage or inflammation (FIG. 24 panel G). Neutrophil infiltration was significantly elevated in PBS-treated mice as determined by MPO activity compared to uninfected mice, and no significant neutrophil infiltration was detectable in the intestines from cTxAB-immunized mice. Thus the data show antibodies induced by cTxAB immunization protected mice against C. difficile toxin-induced mucosal damage and colitis.

CDI has become increasingly difficult to manage in part due to the ineffectiveness of current antibiotic treatments which result in a high rate of relapse/recurrence (Kelly, C P et al. 2008 N Engl J Med 359: 1932; Rupnik, M. H. et al. 2009 Nat Rev Microbiol 7: 526). To evaluate the efficacy of candidate vaccines herein in preventing relapse/recurrence, a CDI relapse/recurrence model in mice was established. The scheme of immunization and disease induction is shown in FIG. 24 panel H. After the primary infection, the surviving mice from the PBS-treated control group developed disease with severity similar to that of the primary CDI. These mice developed severe diarrhea and weight loss and 40% of mice became moribund (FIG. 24 panels J, K and L). The cTxAB immunization in contrast protected mice from relapse. None of these mice developed a sign of disease and 100% survival was observed after the second C. difficile challenge (FIG. 24 panels J, K and L). This protection was further associated with long-lasting immunity seen in cTxAB-immunized mice (FIG. 21 panels J, H and I).

It is desirable for a vaccine to induce rapid protection against CDI in hospitalized patients who undergo treatments that disrupt the gut microflora and therefore put them at a high risk of C. difficile infection. A single immunization of mice with cTxAB induced a measurable antibody response against both toxins (FIG. 21 panel A), and this antibody response was evaluated for sufficient potency to protect mice from CDI. The mice were immunized on the same day as antibiotic treatment and were challenged with 10⁶ UK1 spores 6 days later. Data show that 90% off cTxAB-immunized mice survived. In contrast, nearly half of PBS-control mice became moribund (p<0.05, FIG. 24 panel M). Surviving mice from both groups experienced a similar degree of diarrhea and weight loss (FIG. 24 panel N). Therefore, a single immunization of mice with cTxAB provided significant protection from severe disease and mortality, and not diarrhea or weight loss.

A rapid vaccination scheme was thus evaluated for protection against these symptoms, and from relapse/recurrence. After a first immunization and C. difficile spore challenge, the surviving mice from both groups were again immunized with cTxAB twice before rechallenge with UK1 spores (FIG. 24 panel I). None of cTxAB-immunized mice died or developed relapsing disease and PBS-treated mice suffered with mortality and sharp weight loss with 90% developing diarrhea (FIG. 24 panels O, P and Q).

### Example 24

### Sublingual Immunization of Piglets with aTcdB, with and without mLT

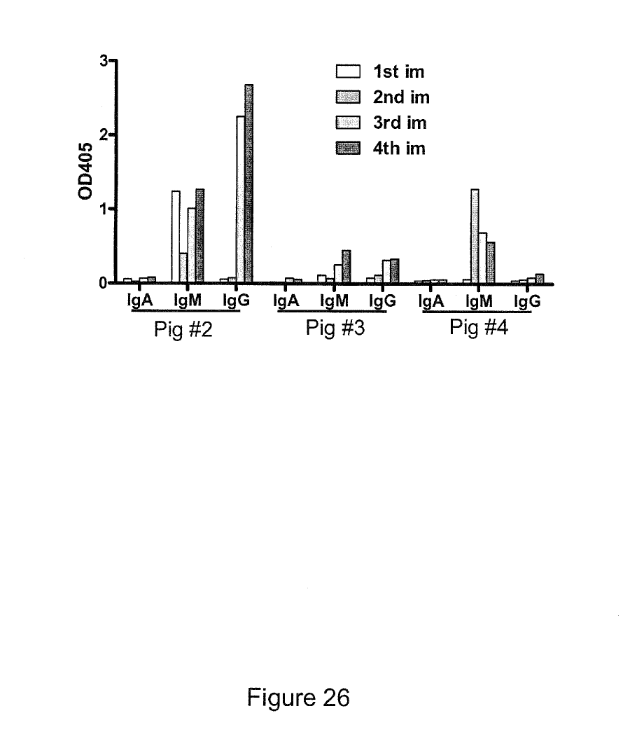

Two week old gnotobiotic (GB) piglets were immunized sublingually (SL) three times every two weeks with either 25 μg of aTcdB mixed with 10 μg of mLT (pigs #1 and #2), or with 25 μg of aTcdB only (pigs #3 and #4). One animal which received aTcdB with mLT (pig #1), was euthanized due to an unrelated illness. FIG. 26 shows the serum antibody response of the three remaining piglets immunized with aTcdB, with (pig #2) or without mLT (pigs #3 and #4).

It was observed that SL immunization of piglet with aTcdB mixed with mLT as adjuvant induced a systemic anti-TcdB IgG response (FIG. 26). These data show that SL immunization is an effective route of administration, and that inclusion of mLT as mucosal adjuvant enhances the immune response to this immunogen.

### Example 25

### The Gnotobiotic (GB) Piglet Model

Effective immunization regimens for stimulating optimal immune response and 100% protection against bacterial challenge are evaluated in GB piglet model. This model is useful to satisfy two animal rule required by FDA; the piglet model in for the impact of immunization on an existing treatment of chronic CDI; protection against establishment of CDI chronic disease; protection against a relapse; a model in which preclinical evaluation of safety and optimization of candidates can be done with a view to identify and confirm the best possible vaccine and regimen of immunization likely to be equally effective for humans.

GB piglets are used as second animal model to evaluate vaccines selected after mouse infection studies. These animals routinely are housed in microbiological isolators throughout the study and fed baby milk formula for six weeks, then weaned to weaner diet when kept longer as indicated in Examples herein, which were conducted under the C. difficile IACUC protocol G861-06.

The gnotobiotic piglets orally infected with C. difficile are expected to display diarrhea and become weak and dehydrated, and moribund. Piglets are monitored at least four times per day (at about 9 a.m., about noon, about 4 p.m. and once between 8 p.m. and 12 a.m.) throughout the duration of the treatment. Piglets observed to be lethargic, unable to move, or more than 5% dehydrated, are rehydrated by parenteral administration of fluid electrolytes and glucose. If severe illness is observed, at least one additional check is made between each of the standard check points following observation of the lethargy, inability to move, more than 5% dehydration. Animals that appear moribund at any time are euthanized.

The piglet model offers a range spectrum of symptoms and severity within the disease, from profoundly acute and lethal to chronic diarrhea with the characteristic pseudomembranous colitis (PMC), with intensity and duration that are manipulated in a controlled laboratory setting. The spectrum of clinical signs, including systemic consequences, is similar to that observed in human cases, making the piglet an attractive model in which to perform preclinical evaluation of vaccine candidates and therapeutic agents. These include a range of systemic consequences of C. difficile infection such as ascites, pleural effusion, cardiopulmonary arrest, liver abscess, and multiple organ dysfunction syndrome, which result in severe and even fatal disease. Immune response plays a role in disease severity, and in these examples, cytokine levels are analyzed in the large intestinal contents. IL-8 concentration in particular, was significantly elevated in the piglets challenged with C. difficile (Steele, J. et al. 2010 J Infect Diseases 201:428).

Preclinical evaluation on the efficacy of the immunization regimens in the piglet model of chronic CDI is performed to assess the ability of immunized piglets to resist an oral challenge with C. difficile. Piglets are derived by cesarian section and maintained inside sterile isolators for the duration of the treatment. They are fed milk diet and handled, monitored, and sampled regularly (McMaster-Baxter, N. L. et al. 2007 Pharmacotherapy 27:1029-1039).

TABLE 1
Outline of generic infection prevention example to evaluate a
C. difficile vaccine candidate administered orally, intranasally,
sublingually or parenterally to protect against CDI (e.g. prior to
hospitalization of surgical patients).

| Group | 1^{st} Vacc | 2^{nd} | 3^{rd} | | |
|---|---|---|---|---|---|
| (# piglets) | (age) | Vacc | Vacc | Challenge* | Euthanasia# |
| Test | 7 days | 21 days | 28 days | 35 days (s027) | ~42/45 days |
| vaccine (7) | | | | | |
| Toxoid (7) | 7 days | 21 days | 28 days | 35 days (s027) | ~42/45 days |
| Placebo (7) | 7 days | 21 days | 28 days | 35 days (s027) | ~42-45 days |
| Control (5) | 7 days | 21 days | 28 days | 35 days | ~42/45 days |
| | | | | (sCD37) | |

*Wild type C. difficile hypervirulent strain 027 (10⁸ spores/pig); the Control strain CD37 (10⁸ spores/pig
#Time of euthanasia depends on intensity of symptoms, if any.

Placebo group of Table 1 receives the vehicle plus adjuvant, if included. The control group is immunized as the test vaccine group but challenged with the control strain, against which groups challenged with strain 027 is compared and measured. Two or more test vaccines are tested in parallel for comparative purposes and to conserve on the number of piglets used in these treatemnts since the control and placebo groups can be shared.

Preclinical evaluation is performed on the ability of any of the above immunization regimens to clear infection and/or reduce severity of symptoms of piglets infected with C. difficile.

TABLE 2
Outline of a generic example for treatment of existing chronic
CDI with vaccination administered orally, intranasally, sublingually
or parenterally (e.g. a patient suffering from hospital acquired CDI).

| Group | | 1^{st} | 2^{nd} | 3^{rd} | |
|---|---|---|---|---|---|
| (# piglets) | Challenge* | Vacc | Vacc | Vacc | Euthanasia# |
| Test | 7 days (s027) | 14 days | 28 days | 35 days | ~42/45 days |
| vaccine (7) | | | | | |
| Toxoid (7) | 7 days (s027) | 14 days | 28 days | 35 days | ~42/45 days |
| Placebo (7) | 7 days (s027) | 14 days | 28 days | 35 days | ~42-45 days |
| Control (5) | 7 days (sCD37) | 14 days | 28 days | 35 days | ~42/45 days |

*Wild type C. difficile hypervirulent strain 027 (10⁶ spores/pig); the control strain is CD37 (10⁶ spores/pig)
#Time of euthanasia depends on outcome of vaccination. Placebo group receives the vehicle plus adjuvant, as appropriate. A plurality of test vaccines are tested in parallel for the reasons indicated above.

Piglets are maintained for additional three months after the oral challenge with C. difficile, to monitor the levels of specific serum and secretory antibody, after which they are challenged with C. difficile a second time to assess protection against recurrence/relapse; if the specific antibody levels are deemed low, a booster immunization is given before the second challenge

TABLE 3
Outline of a generic example to evaluate vaccine candidates for
relapse of CDI three months after recovery from C. difficile
challenge.

| | 1^{st} | | | | |
|---|---|---|---|---|---|
| Group | Vacc | 2^{nd} | 3^{rd} | | Re- |
| (# piglets) | (age) | Vacc | Vacc | Challenge* | challenge# |
| Test | 7 days | 21 days | 28 days | 35 days (s027) | ~100 days |
| vaccine (7) | | | | | |
| Toxoid (7) | 7 days | 21 days | 28 days | 35 days (s027) | ~100 days |
| Placebo (7) | 7 days | 21 days | 28 days | 35 days (s027) | ~100 days |
| Control (5) | 7 days | 21 days | 28 days | 35 days (sCD37) | ~100 days |

*Wild type C. difficile hypervirulent strain 027 (10⁸ spores/pig); the control strain is CD37 (10⁸ spores/pig)
#At 100 days sera and fecal Ig are measured and if judged to be low, a booster vaccine is given a week before the second challenge. Time of euthanasia depends on the clinical outcome among the four groups. Placebo group receives the vehicle plus adjuvant, if included. More than one test vaccine will be included in each set of treatments.

Piglets are observed at least twice daily for symptoms ranked 0 to 3: 0 indicates no diarrhea; 1 indicates mild diarrhea; 2 indicates moderate diarrhea; 3 indicates severe diarrhea. The following symptoms are monitored: diarrhea—watery, intermittent, pasty; dehydration determined by skin elasticity and overall appearance; body weight, loss or gain measured three times per week, reflecting health status; anorexia, eagerness to drink, amount of milk consumption per day, alertness, depression, reluctance to move, hunched, squealing when picked up; general appearance, ruffled coat, dirty perineal region; and telemetry monitoring, charted record of vital signs and body temperature fluctuations, respiration and heart rate (see below under animal husbandry).

In cases of serious dehydration, piglets are rehydrated using Aminosyn II 3.5% M combined with 5% Dex Inj NTRMX IP twice daily (20 to 30 ml/injection) until rehydration is restored. The clinical, histological, bacterial count and inflammatory (cytokine levels) responses of each individual within each of the four groups were ranked from 0 to 3, with 0 denoting no impact, according to severity for each individual animal, and recorded observations were entered into the database.

The comparison between the four groups reflects the impact of the vaccination on the course of the clinical manifestation after bacterial challenge and includes analysis of mucosal lesions at necropsy. A ranking of 1 to 3 is assigned to each organ where changes are observed macroscopically or microscopically, with zero indicating normal or no change in the parameters measured in group 4. Conversion of data into numerical figures permits a development of a scoring system that is treated with appropriate statistical analysis for clinical observations and necropsy finding. Clinical observations include six parameters such as diarrhea, anorexia, dehydration, alertness, telemetry and body weight. Daily scores in this category vary from 0 to a maximum of 18 (3×6). Necropsy findings include parameters: gut lesions, inflammation, bacterial count, systemic toxemia, visceral abnormalities, clinical pathology. Scores in this category varied from 0 to 18 (3×6). According to this scoring system an ideal control group 4 score would be 0; the placebo group 4 score would approach 18 for the clinical observations, and 18 for the necropsy finding. The magnitude of the scores for groups 1 and 2 would be used to establish protective parameters. Thus scores that are closer to 18 indicate poor efficacy, closer to zero reflect great efficacy.

In addition to the numerical scoring system described above, a comprehensive and detailed description of macroscopic and microscopic observations were recorded and entered into the database which provided additional and more detailed information which was further analyzed separately.

### Example 26

### PLG Polymers for Oral Immunization

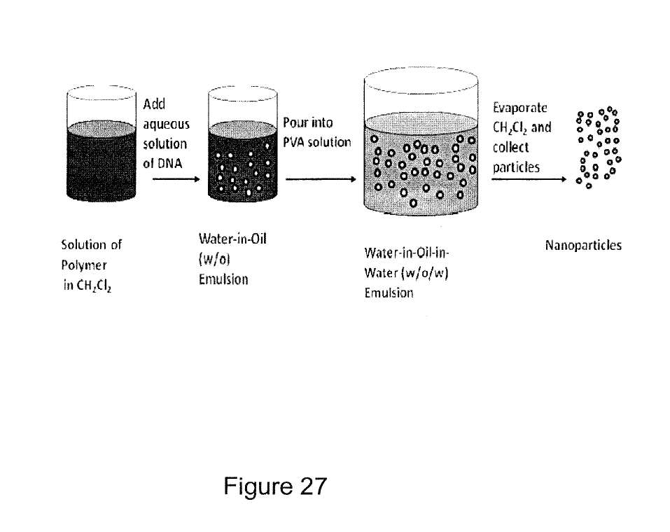

The aTxAB and cTxAB proteins are encapsulated in PLG by methods shown in FIG. 27. Medisorb PLG polymers (Alkermes, Inc., Cincinnati, Ohio) are used in the method herein, prepared as oil-in-water emulsions and blended with a shear-type mixer such as that produced by Silverson Machines, East Longmeadow, Mass., by the general procedure for preparing the vaccines in FIG. 6 (Herrmann, J. E. et al. 1999 Virology 259:148-153). The particles generated by this method are generally less than 5 μm in size. By manipulating the procedure nanoparticles consistently in the 100 nm to 250 nm range are also produced. Nanoparticles increase uptake of encapsulated vaccines administered by each of the two mucosal routes, intranasal and oral. Studies on the uptake of PLG microparticles have shown that following oral administration to mice, PLG microparticles 1 μm to 10 μm were taken up into the Peyer's patches of the gut-associated lymphoid tissue. Particles larger or equal in size to about 5 μm that were taken up remained localized for up to 35 days, and the particles lesser in size than about 5 μm were disseminated within macrophages, mesenteric lymph nodes, blood circulation, and spleen (Eldridge, J. H. et al. 1989 Curr Top Microbiol Immunol 146:59-66; Eldridge, J. H. et al. 1989 Adv Exp Med Biol 251:191-202). PLG microparticles are not selectively targeted to M cells, but nonspecific binding to M cells and subsequent transcytosis has been shown in rabbits (Jepson, M. A. et al. 1996 Pflugers Arch 432:225-233; Jepson, M. A. et al. 1993 J Drug Target 1:245-249). It has been shown that PLG microparticles containing antigen bind to and are transported by M cells in a similar manner to that found with empty PLG microparticles (O'Hagan, D. T. 1996 J Anat 189 (Pt 3):477-482). Uptake of bovine serum albumin encapsulated in PLG microparticles by Peyer's patches has been shown in a rat model (Desai, M. P. et al. 1996 Pharm Res 13:1838-1845). The encapsulated aTxAB or cTxAB were administered using gavage.

The following claims are exemplary only and are not to be construed as further limiting. One of ordinary skill in the art would readily determine from the examples and claims numerous equivalents that are within the scope of the invention herein.

What is claimed is:

1. A vaccine composition comprising an atoxic recombinant Clostridium toxin protein for immunizing, the protein comprising a glucosyltransferase domain (GT), a cysteine proteinase domain (CPD), a transmembrane domain (TMD), and a receptor binding domain (RBD), wherein the domains are operably linked to a protein purification tag located at a C-terminus, wherein the protein is produced recombinantly in a Bacillus host.

2. The composition according to claim 1, wherein amino acid sequences of the domains of the Clostridium toxin protein are obtained from a strain selected from at least one from the group of: C. difficile, C. perfringens, C. sordellii, C. septicum, C. tertium, C. botulinum, and the like.

3. The composition according to claim 2, wherein the atoxic recombinant protein comprises a mutation in at least one C. difficile protein selected from the group of a TcdA protein and a TcdB protein, and retains native protein conformation, wherein toxicity of the protein is reduced at least: about 10-fold to about 1,000-fold, or about 1,000-fold to about 10,000-fold, or about 10,000-fold to about 10 million-fold compared to wild-type Clostridium toxin.

4. The composition according to claim 3, wherein the mutation is located in the GT domain of the TcdA protein and the TcdB protein.

5. The composition according to claim 3, wherein the atoxic recombinant protein comprises a chimeric fusion cTxAB having a first amino acid sequence derived from the TcdA protein and a second amino acid sequence derived from the TcdB protein.

6. The composition according to claim 5, wherein the first amino acid sequence comprises the TcdA RBD domain and the second amino acid sequence comprises the TcdB GT, CPD and TMD domains, the atoxic recombinant protein further comprising a protease cleavage site for removal of the purification tag.

7. The composition according to claim 6, wherein the purification tag is at least one selected from the group of: Arg-tag, calmodulin-binding peptide, cellulose-binding domain, DsbA, c-myc-tag, glutathione S-transferase, FLAG-tag, HAT-tag, His-tag, maltose-binding protein, NusA, S-tag, SBP-tag, Strep-tag, and thioredoxin.

8. The composition according to claim 1, wherein the Bacillus is Bacillus megaterium.

9. The composition according to claim 1 in an effective dose.

10. The composition according to claim 1 further comprising at least one of an adjuvant and a pharmaceutically acceptable carrier.

11. A nucleic acid encoding the protein according to claim 1.

12. The composition according to claim 11, wherein the nucleic acid is operably linked to a vector.

13. A kit comprising a container, a composition or nucleic acid according to any of claims 1-12, and instructions for use.

14. A method of eliciting an immune response specific for a Clostridium difficile toxin in a subject, the method comprising:
engineering a nucleic acid encoding an atoxic mutation of a C. difficile toxin protein, wherein the protein comprises a glucosyltransferase domain (GT), a cysteine proteinase domain (CPD), a transmembrane domain (TMD), a receptor binding domain (RBD), and a purification tag located at a C-terminus;
expressing the protein in a cell, purifying the protein, and removing the purification tag; and,
formulating the protein and contacting the subject with the protein or with the nucleic acid, thereby eliciting in the subject at least one of a humoral immune response and a cell-mediated immune response specific to the protein.

15. The method according to claim 14, wherein engineering comprises obtaining the mutation in at least one of: a TcdA nucleic acid sequence encoding an amino acid sequence from a C. difficile TcdA protein, and a TcdB nucleic acid sequence encoding an amino acid sequence from a C. difficile TcdB protein.

16. The method according to claim 15, wherein the mutation is located in the GT domain of the at least one of the TcdA protein and the TcdB protein, wherein engineering the mutation comprises introducing an amino acid substitution or an amino acid deletion into the TcdA nucleic acid sequence or the TcdB nucleic acid sequence, or wherein engineering the toxin protein comprises introducing a plurality of mutations.

17. The method according to claim 16, wherein the substitution mutation comprises replacing a tryptophan with an alanine and replacing an aspartic acid with an asparagine.

18. The method according to claim 14, wherein the protein comprises an atoxic chimeric protein cTxAB having a TcdA amino an acid sequence derived from a TcdA protein and a TcdB amino acid sequence derived from a TcdB protein, wherein engineering the amino acid sequence comprises recombinantly joining nucleic acids encoding the RBD domain from the TcdA protein with that encoding the amino acid sequence of the GT, CPD and TMD domains of the TcdB protein, wherein the protein domains are operably linked to a purification tag located at the C-terminus and a protease cleavage site for removal of the tag.

19. The method according to claim 18, wherein engineering the TMD domain comprises deleting at least one aspartic acid.

20. The method according to claim 14, wherein contacting the subject further comprises administering the protein by a route selected from at least one of the group consisting of intravenous, intramuscular, intraperitoneal, intradermal, mucosal, subcutaneous, sublingual, intranasal and oral.

21. The method according to claim 14, further comprising analyzing an antibody titer in serum of the subject, and observing an increase in antibody that specifically binds a Clostridium antigen compared to prior to control serum obtained prior to contacting, or compared to that in a control not so contacted, wherein the immune response is elicited.

22. A method of producing a recombinant mutant Clostridium toxin protein, the method comprising:
constructing a nucleic acid vector encoding a gene for the Clostridum protein, wherein the protein comprises a glucosyltransferase domain (GT), a cysteine proteinase domain (CPD), a transmembrane domain (TMD), a receptor binding domain (RBD), the gene being operably linked to regulatory signals for expressing the gene in a cell and to a selectable marker and to a purification tag located at a C-terminus;
contacting a protoplast of the cell with the vector under conditions suitable to transformation or transduction of the cell; and,
selecting a transformant carrying the selectable marker and expressing the recombinant mutant Clostridium toxin protein.

23. The method according to claim 22, wherein the cell is selected from the group of: B. megaterium, B. subtilis, B. thuringiensis, B. cereus, and B. licheniformis, or wherein the Clostridium is selected from at least one from the group of: C. difficile, C. perfringens, C. sordellii, C. septicum, C. tertium, C. botulinum, and the like.

24. The method according to claim 22, wherein constructing the nucleic acid vector comprises combining a first nucleic acid sequence encoding an atoxic mutant C. difficile TcdA protein and a second nucleic acid sequence encoding an atoxic mutant C. difficile TcdB protein.

25. The method according to claim 24, wherein the recombinant mutant Clostridium toxin protein comprises at least one mutation, wherein the at least one mutation comprises a substitution or a deletion of at least one amino acid.

26. The method according to claim 25, wherein the at least one mutation is located in the GT domain.

27. The method according to claim 22, wherein the gene encodes a recombinant chimeric cTxAB protein comprising a TcdB amino acid sequence derived from the TcdB protein and a TcdA amino acid sequence derived from the TcdA protein, wherein the TcdB protein amino acid sequence comprises the GT domain and the TcdA protein amino acid sequence comprises the RBD, CPD and TMD domains, the protein comprises a protease cleavage site for removal of the purification tag.

28. The method according to claim 22, wherein the gene encodes a recombinant chimeric TxB-Ar protein comprising a TcdA amino acid sequence derived from the TcdA protein and a TcdB amino acid sequence derived from the Tcd B protein, wherein the TcdA protein amino acid sequence comprises the RBD domain and the TcdB protein amino acid sequence comprises the GT, CPD and TMD domains, wherein protein domains are operably linked to a purification tag with a protease cleavage site for removal of the tag.


SEQUENCE LISTING (6 sequences)

SEQ ID NO: 1 - DNA, 22 bp, Artificial Sequence
  The sequence was designed and synthesized
tttgtttatccaccgaactaag

SEQ ID NO: 2 - DNA, 22 bp, Artificial Sequence
  The sequence was designed and synthesized
tcttcagaaagggatccaccag

SEQ ID NO: 3 - DNA, 22 bp, Artificial Sequence
  The sequence was designed and synthesized
tggtggatccctttctgaagac

SEQ ID NO: 4 - DNA, 18 bp, Artificial Sequence
  The sequence was designed and synthesized
actgctccagtttcccac

SEQ ID NO: 5 - DNA, 32 bp, Artificial Sequence
  The sequence was designed and synthesized
aattaccggttttaacttagtaactggatggc

SEQ ID NO: 6 - DNA, 45 bp, Artificial Sequence
  The sequence was designed and synthesized
aattgcatgctggtaccctccatatatcccaggggcttttactcc